(19) United States
(12) Patent Application Publication
(10) Pub. No.: US 2023/0083487 A1
(43) Pub. Date: Mar. 16, 2023

(54) TREATMENT OF LUNG CANCER USING A COMBINATION OF AN ANTI-PD-1 ANTIBODY AND ANOTHER ANTI-CANCER AGENT

(71) Applicant: Bristol-Myers Squibb Company, Princeton, NJ (US)

(72) Inventor: Faith E. NATHAN, Princeton, NJ (US)

(73) Assignee: Bristol-Myers Squibb Company, Princeton, NJ (US)

(21) Appl. No.: 17/818,298

(22) Filed: Aug. 8, 2022

Related U.S. Application Data
(63) Continuation of application No. 16/073,676, filed on Jul. 27, 2018, now abandoned.
(60) Provisional application No. 62/287,717, filed on Jan. 27, 2016.

(51) Int. Cl. C07K 16/28; A61P 35/00

(52) U.S. Cl. CPC C07K 16/2818; A61P 35/00; A61K 2039/507

(57) ABSTRACT

This disclosure provides a method for treating a subject afflicted with a lung cancer, which method comprises administering to the subject therapeutically effective amounts of: (a) an anti-cancer agent which is an antibody or an antigen-binding portion thereof that specifically binds to a Programmed Death-1 (PD-1) receptor and inhibits PD-1 activity which can be administered by infusion for less than 60 minutes; and, optionally, (b) another anti-cancer agent which is administered by infusion for less than 90 minutes. The other anti-cancer agent can be an anti-Cytotoxic T-Lymphocyte Antigen-4 (CTLA-4) antibody.

### CROSS REFERENCE TO RELATED APPLICATIONS

This application is a continuation of U.S. application Ser. No. 16/073,676, which is the U.S. National Phase application of International Application No. PCT/US2017/015333 filed on Jan. 27, 2017, which claims the benefit of U.S. Provisional Application No. 62/287,717 filed on Jan. 27, 2016, each of which is incorporated by reference herein in its entirety.

### FIELD OF THE INVENTION

This invention relates to methods for treating lung cancer in a subject comprising administering to the subject a combination of an anti-cancer agent which is an anti-Programmed Death-1 (PD-1) antibody and, optionally, another anti-cancer agent.

### BACKGROUND OF THE INVENTION

Human cancers harbor numerous genetic and epigenetic alterations, generating neoantigens potentially recognizable by the immune system (Sjoblom et al., 2006). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

Until recently, cancer immunotherapy had focused substantial effort on approaches that enhance anti-tumor immune responses by adoptive-transfer of activated effector cells, immunization against relevant antigens, or providing non-specific immune-stimulatory agents such as cytokines. In the past decade, however, intensive efforts to develop specific immune checkpoint pathway inhibitors have begun to provide new immunotherapeutic approaches for treating cancer, including the development of an antibody (antibody), ipilimumab (YERVOY®), that binds to and inhibits CTLA-4 for the treatment of patients with advanced melanoma (Hodi et al., 2010) and the development of antibodies such as nivolumab and pembrolizumab (formerly lambrolizumab; USAN Council Statement, 2013) that bind specifically to the Programmed Death −1 (PD-1) receptor and block the inhibitory PD-1/PD-1 ligand pathway (Topalian et al., 2012a, b; Topalian et al., 2014; Hamid et al., 2013; Hamid and Carvajal, 2013; McDermott and Atkins, 2013).

PD-1 is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to downregulate T cell activation and cytokine secretion upon binding to PD-1. Inhibition of the PD-1/PD-L1 interaction mediates potent antitumor activity in preclinical models (U.S. Pat. Nos. 8,008,449 and 7,943,743), and the use of antibody inhibitors of the PD-1/PD-L1 interaction for treating cancer has entered clinical trials (Brahmer et al., 2010; Topalian et al., 2012a; Topalian et al., 2014; Hamid et al., 2013; Brahmer et al., 2012; Flies et al., 2011; Pardoll, 2012; Hamid and Carvajal, 2013).

Nivolumab (formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014). Nivolumab has shown activity in a variety of advanced solid tumors, including renal cell carcinoma (renal adenocarcinoma, or hypernephroma), melanoma, and non-small cell lung cancer (NSCLC) (Topalian et al., 2012a; Topalian et al., 2014; Drake et al., 2013; WO 2013/173223).

Ipilimumab (YERVOY®) is a fully human, IgG1 monoclonal antibody that blocks the binding of CTLA-4 to its B7 ligands, thereby stimulating T cell activation and improving overall survival (OS) in patients with advanced melanoma (Hodi et al., 2010). Concurrent therapy with nivolumab and ipilimumab in a Phase 1 clinical trial produced rapid and deep tumor regression in a substantial proportion of patients with advanced melanoma, and was significantly more effective than either antibody alone (Wolchok et al., 2013; WO 2013/173223). However, it was hitherto not known whether this combination of immunoregulatory antibodies would be similarly effective in other tumor types.

NSCLC is the leading cause of cancer death in the U.S. and worldwide (NCCN GUIDELINES®, 2013— Non-Small Cell Lung Cancer). NSCLCs are relatively insensitive to chemotherapy but patients with Stage IV disease who have a good performance status (PS) benefit from treatment with chemotherapeutic drugs, including platinum agents (e.g., cisplatin, carboplatin), taxanes agents (e.g., paclitaxel, albumin-bound paclitaxel, docetaxel), vinorelbine, vinblastine, etoposide, pemetrexed and gemcitabine, and various combinations of these drugs.

### SUMMARY OF THE INVENTION

The present disclosure provides a method for treating a subject afflicted with a lung cancer comprising administering to the subject a therapeutically effective amounts of: (a) an antibody or an antigen-binding portion thereof that specifically binds to and inhibits PD-1; and, optionally, (b) an antibody or an antigen-binding portion thereof that specifically binds to and inhibits CTLA-4. In some embodiments, the anti-cancer agent which is an antibody or an antigen-binding portion thereof that binds specifically to a Programmed Death-1 (PD-1) receptor and inhibits PD-1 activity is administered by infusion for less than 60 minutes (e.g., about 30 minutes). In some embodiments, the other anti-cancer agent is administered by infusion for less than 90 minutes (e.g., about 60 or about 30 minutes). In preferred embodiments, the lung cancer is non-small cell lung cancer (NSCLC). In certain preferred embodiments of any of the therapeutic methods disclosed herein, the anti-PD-1 antibody is nivolumab. In other embodiments, the anti-PD-1 antibody is pembrolizumab. In certain other preferred embodiments of any of the therapeutic methods disclosed herein, the anti-CTLA-4 antibody is ipilimumab. In other embodiments, the anti-CTLA-4 antibody is tremelimumab.

In certain embodiments, the subject has been pre-treated for the lung cancer. In other embodiments, the lung cancer is an advanced, metastatic and/or refractory cancer. In preferred embodiments, the administration of the combination of the antibody or antigen-binding portion thereof and the other anti-cancer agent induces a durable clinical response in the subject.

The disclosure also provides a kit for treating a subject afflicted with a lung cancer, the kit comprising: (a) a dosage ranging from 0.1 to 10 mg/kg body weight of an anti-cancer agent which is an antibody or an antigen-binding portion thereof that specifically binds to the PD-1 receptor and inhibits PD-1 activity; (b) a dosage of another anti-cancer agent which is a dosage ranging from 0.1 to 10 mg/kg body weight of an antibody or an antigen-binding portion thereof that specifically binds to and inhibits CTLA-4; and (c) instructions for using the anti-PD-1 antibody and the other anti-cancer agent for treating the subject.

The present invention also includes a method for treating a subject afflicted with a lung cancer comprising administering to the subject a flat dose of a therapeutically effective amount of an anti-cancer agent which is an antibody or an antigen-binding portion thereof that binds specifically to a Programmed Death-1 (PD-1) receptor and inhibits PD-1 activity. In some embodiments, the flat dose of an anti-PD-1 antibody or an antigen-binding portion thereof is a dose higher than 240 mg. In other embodiments, the flat dose is administered every 2 weeks. In certain embodiments, the flat dose is at least about 480 mg. In some embodiments, the flat dose is administered every 4 weeks.

Other features and advantages of the instant invention will be apparent from the following detailed description and examples which should not be construed as limiting. The contents of all cited references, including scientific articles, newspaper reports, GenBank entries, patents and patent applications cited throughout this application are expressly incorporated herein by reference.

### Embodiments

E1. A method for treating a subject afflicted with a lung cancer comprising administering to the subject a therapeutically effective amounts of:

(a) an anti-cancer agent which is an antibody or an antigen-binding portion thereof that binds specifically to a Programmed Death-1 (PD-1) receptor and inhibits PD-1 activity which is administered by infusion for less than 60 minutes; and
(b) optionally, another anti-cancer agent which is administered by infusion for less than 90 minutes.

E2. The method of embodiment E1, wherein the lung cancer is non-small cell lung cancer (NSCLC).

E3. The method of embodiment E2, wherein the NSCLC has a squamous histology.

E4. The method of embodiment E2, wherein the NSCLC has a non-squamous histology.

E5. The method of any one of embodiments E1 to E4, wherein the anti-PD-1 antibody or antigen-binding portion thereof cross-competes with nivolumab for binding to human PD-1.

E6. The method of any one of embodiments E1 to E5, wherein the anti-PD-1 antibody or antigen-binding portion thereof is a chimeric, humanized or human monoclonal antibody or a portion thereof.

E7. The method of any one of embodiments E1 to E5, wherein the anti-PD-1 antibody or antigen-binding portion thereof comprises a heavy chain constant region which is of a human IgG1 or IgG4 isotype.

E8. The method of any one of embodiments E1 to E7, wherein the anti-PD-1 antibody is nivolumab.

E9. The method of any one of embodiments E1 to E7, wherein the anti-PD-1 antibody is pembrolizumab.

E10. The method of any one of embodiments E1 to E9, wherein the anti-PD-1 antibody or antigen-binding portion thereof is administered at a dose ranging from 0.1 to 10.0 mg/kg body weight once every 2 or 3 weeks.

E11. The method of embodiment E10, wherein the anti-PD-1 antibody or antigen-binding portion thereof is administered at a dose of 1 or 3 mg/kg body weight once every 2 weeks or once every 3 weeks.

E12. The method of embodiment E11, wherein the anti-PD-1 antibody or antigen-binding portion thereof is administered at a dose of 1 mg/kg body weight once every 3 weeks.

E13. The method of embodiment E11, wherein the anti-PD-1 antibody or antigen-binding portion thereof is administered at a dose of 3 mg/kg body weight once every 2 weeks.

E14. The method of any one of embodiments E1 to E13, wherein the anti-PD-1 antibody or antigen-binding portion is administered for as long as clinical benefit is observed or until unmanageable toxicity or disease progression occurs.

E15. The method of any one of embodiments E1 to E14, wherein the other anti-cancer agent is an antibody or an antigen-binding portion thereof that binds specifically to Cytotoxic T-Lymphocyte Antigen-4 (CTLA-4) and inhibits CTLA-4 activity.

E16. The method of embodiment E15, wherein the anti-CTLA-4 antibody or antigen-binding portion thereof cross-competes with ipilimumab for binding to human CTLA-4.

E17. The method of embodiment E15, wherein the anti-CTLA-4 antibody or antigen-binding portion thereof is a chimeric, humanized or human monoclonal antibody or a portion thereof.

E18. The method of any one of embodiments E15 to E17, wherein the anti-CTLA-4 antibody or antigen-binding portion thereof comprises a heavy chain constant region which is of a human IgG1 isotype.

E19. The method of any one of embodiments E15 to E18, wherein the anti-CTLA-4 antibody is ipilimumab.

E20. The method of any one of embodiments E15 to E18, wherein the anti-CTLA-4 antibody is tremelimumab.

E21. The method of any one of embodiments E15 to E20, comprising:

(a) an induction phase, wherein the anti-PD-1 and anti-CTLA-4 antibodies or antigen-binding portions thereof are administered in combination in 2, 4, 6, 8 or 10 doses, each dose ranging from 0.1 to 10.0 mg/kg body weight administered at least once every 2, 3, or 4 weeks; followed by
(b) a maintenance phase, wherein no anti-CTLA-4 antibody or antigen-binding portion thereof is administered and the anti-PD-1 antibody or antigen-binding portion thereof is repeatedly administered at a dose ranging from 0.1 to 10 mg/kg at least once every 2, 3 or 4 weeks.

E22. The method of embodiment E21, wherein:

(a) the induction phase comprises combination doses administered at 3-week intervals, wherein:
(i) the anti-PD-1 antibody or antigen-binding portion thereof is administered at 3 mg/kg body weight and the anti-CTLA-4 antibody or antigen-binding portion thereof is administered at 1 mg/kg body weight;
(ii) the anti-PD-1 antibody or antigen-binding portion thereof is administered at 1 mg/kg body weight and the anti-CTLA-4 antibody or antigen-binding portion thereof is administered at 3 mg/kg body weight;
(iii) the anti-PD-1 antibody or antigen-binding portion thereof is administered at 1 mg/kg body weight and the anti-CTLA-4 antibody or antigen-binding portion thereof is administered at 1 mg/kg body weight; or
(iv) the anti-PD-1 antibody or antigen-binding portion thereof is administered at 3 mg/kg body weight and the anti-CTLA-4 antibody or antigen-binding portion thereof is administered at 3 mg/kg body weight; and
   (b) the maintenance phase comprises repeated administration of the anti-PD-1 antibody or antigen-binding portion thereof at a dose of 3 mg/kg every 2 weeks for as long as clinical benefit is observed or until unmanageable toxicity or disease progression occurs.

E23. The method of any one of embodiments E15 to E23, wherein the anti-PD-1 and anti-CTLA-4 antibodies are formulated for intravenous administration.

E24. The method of embodiment E22, wherein the anti-PD-1 antibody or antigen-binding portion thereof and the anti-CTLA-4 antibody or antigen-binding portion thereof are administered sequentially to the subject during the induction phase.

E25. The method of embodiment E24, wherein the anti-PD-1 and anti-CTLA-4 antibodies are administered within 30 minutes of each other.

E26. The method of embodiment E25, wherein

(a) the anti-PD-1 antibody or antigen-binding portion thereof is administered before the anti-CTLA-4 antibody or antigen-binding portion thereof; or
(b) the anti-CTLA-4 antibody or antigen-binding portion thereof is administered before the anti-PD-1 antibody or antigen-binding portion thereof.

E27. The method of any one of embodiments E15 to E26, wherein the anti-PD-1 antibody or antigen-binding portion thereof and the anti-CTLA-4 antibody or antigen-binding portion thereof are administered concurrently in separate compositions.

E28. The method of any one of embodiments E15 to E26, wherein the anti-PD-1 antibody or antigen-binding portion thereof and the anti-CTLA-4 antibody or antigen-binding portion thereof are admixed as a single composition for concurrent administration.

E29. The method of any one of embodiments E1 to E28, wherein the anti-PD-1 antibody or antigen-binding portion thereof is administered at a subtherapeutic dose.

E30. The method any one of embodiments E15 to E29, wherein the anti-CTLA-4 antibody or antigen-binding portion thereof is administered at a subtherapeutic dose.

E31. The method any one of embodiments E15 to E29, wherein the anti-PD-1 antibody or antigen-binding portion thereof and the anti-CTLA-4 antibody or antigen-binding portion thereof are each administered at a subtherapeutic dose.

E32. The method any one of embodiments E21 to E31, wherein the administration of the anti-PD-1 antibody in the maintenance phase is continued for as long as clinical benefit is observed or until unmanageable toxicity or disease progression occurs.

E33. The method of any one of embodiments E1 to E32, wherein the subject has a PD-L1+ tumor.

E34. The method of any one of embodiments E1 to E32, wherein the subject has a PD-L1- tumor.

E35. The method of any one of embodiments E1 to E34, wherein the subject does not have an EGFR mutation or an ALK translocation.

E36. The method of any one of embodiments E1 to E35, which is administered as a second-line therapy wherein the subject has progression on or after platinum-based chemotherapy.

E37. The method of any one of embodiments E1 to E36, wherein the anti-cancer agent which is an antibody or an antigen-binding portion thereof that binds specifically to a Programmed Death-1 (PD-1) receptor and inhibits PD-1 activity is administered by infusion for about 30 minutes.

E38. The method of any one of embodiments E1 to E37, wherein the other anti-cancer agent is administered by infusion for about 30 minutes.

E39. The method of any one of embodiments E1 to E9, E14 to E20, or E23 to E38, wherein the anti-PD-1 antibody or antigen-binding portion thereof is administered at a flat dose.

E40. The method of embodiment E39, wherein the flat dose is at least about 240 mg.

E41. The method of embodiment E39 or E40, wherein the flat dose is administered every 2 weeks.

E42. The method of embodiment E39, wherein the flat dose is at least about 480 mg.

E43. The method of embodiment E39 or E42, wherein the flat dose is administered every 4 weeks.

E44. A method for treating a subject afflicted with a lung cancer comprising administering to the subject a flat dose of a therapeutically effective amount of an anti-cancer agent which is an antibody or an antigen-binding portion thereof that binds specifically to a Programmed Death-1 (PD-1) receptor and inhibits PD-1 activity.

E45. The method of embodiment E44, wherein the flat dose is a dose higher than 240 mg.

E46. The method of embodiment E44 or E45, wherein the flat dose is administered every 2 weeks.

E47. The method of embodiment E44, wherein the flat dose is at least about 480 mg.

E48. The method of embodiment E44 or E47, wherein the flat dose is administered every 4 weeks.

E49. A kit for treating a subject afflicted with a lung cancer, the kit comprising:

(a) a flat dosage higher than 240 mg of an antibody or an antigen-binding portion thereof that specifically binds to the PD-1 receptor and inhibits PD-1 activity; and (b) instructions for using the anti-PD-1 antibody in the method of any of embodiments E39 to E48.

E50. A kit for treating a subject afflicted with a lung cancer, the kit comprising:

(a) a dosage ranging from 0.1 to 10 mg/kg body weight of an anti-cancer agent which is an antibody or an antigen-binding portion thereof that specifically binds to the PD-1 receptor and inhibits PD-1 activity;

(b) a dosage of another anti-cancer agent which is a dosage ranging from 0.1 to 10 mg/kg body weight of an antibody or an antigen-binding portion thereof that specifically binds to and inhibits CTLA-4; and

(c) instructions for using the anti-PD-1 antibody and the other anti-cancer agent in the method of any of embodiments E1 to E38.

### BRIEF DESCRIPTION OF THE DRAWINGS

FIG. 1 shows a study design schematic for an Open-Label, Randomized Phase 3 Trial of nivolumab versus platinum doublet chemotherapy and nivolumab plus ipilimumab versus platinum doublet chemotherapy in Subjects with Chemotherapy-Naïve Stage IV or Recurrent Non-Small Cell Lung Cancer (NSCLC).

FIG. 2 shows a study design schematic for a dose frequency optimization, Phase IIIb/IV trial of nivolumab 240 mg every 2 weeks versus nivolumab 480 mg every 4 weeks in subjects with advanced or metastatic non-small cell lung cancer who received 4 months of nivolumab at 3 mg/kg or 240 mg every 2 weeks.

### DETAILED DESCRIPTION OF THE INVENTION

The present invention relates to methods for treating a lung cancer patient comprising administering to the patient a combination of an anti-PD-1 antibody and another anti-cancer agent.

### Terms

In order that the present disclosure may be more readily understood, certain terms are first defined. As used in this application, except as otherwise expressly provided herein, each of the following terms shall have the meaning set forth below. Additional definitions are set forth throughout the application.

“Administering” refers to the physical introduction of a composition comprising a therapeutic agent to a subject, using any of the various methods and delivery systems known to those skilled in the art. Preferred routes of administration for the anti-PD-1 antibody include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. The TKI is typically administered via a non-parenteral route, preferably orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

An “adverse event” (AE) as used herein is any unfavorable and generally unintended or undesirable sign (including an abnormal laboratory finding), symptom, or disease associated with the use of a medical treatment. For example, an adverse event may be associated with activation of the immune system or expansion of immune system cells (e.g., T cells) in response to a treatment. A medical treatment may have one or more associated AEs and each AE may have the same or different level of severity. Reference to methods capable of “altering adverse events” means a treatment regime that decreases the incidence and/or severity of one or more AEs associated with the use of a different treatment regime.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region is comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

An immunoglobulin may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman antibody may be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

An “isolated antibody” refers to an antibody that is substantially free of other antibodies having different antigenic specificities (e.g., an isolated antibody that binds specifically to PD-1 is substantially free of antibodies that bind specifically to antigens other than PD-1). An isolated antibody that binds specifically to PD-1 may, however, have cross-reactivity to other antigens, such as PD-1 molecules from different species. Moreover, an isolated antibody may be substantially free of other cellular material and/or chemicals.

The term “monoclonal antibody” (“mAb”) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. MAbs may be produced by hybridoma, recombinant, transgenic or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the invention can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

A “humanized antibody” refers to an antibody in which some, most or all of the amino acids outside the CDR domains of a non-human antibody are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen” antibody refers to an antibody that binds specifically to the antigen. For example, an anti-PD-1 antibody binds specifically to PD-1 and an anti-CTLA-4 antibody binds specifically to CTLA-4.

An “antigen-binding portion” of an antibody (also called an “antigen-binding fragment”) refers to one or more fragments of an antibody that retain the ability to bind specifically to the antigen bound by the whole antibody.

A “cancer” refers a broad group of various diseases characterized by the uncontrolled growth of abnormal cells in the body. Unregulated cell division and growth divide and grow results in the formation of malignant tumors that invade neighboring tissues and may also metastasize to distant parts of the body through the lymphatic system or bloodstream.

“Cytotoxic T-Lymphocyte Antigen-4 (CTLA-4)” refers to an immunoinhibitory receptor belonging to the CD28 family. CTLA-4 is expressed exclusively on T cells in vivo, and binds to two ligands, CD80 and CD86 (also called B7-1 and B7-2, respectively). The term “CTLA-4” as used herein includes human CTLA-4 (hCTLA-4), variants, isoforms, and species homologs of hCTLA-4, and analogs having at least one common epitope with hCTLA-4. The complete hCTLA-4 sequence can be found under GenBank Accession No. AAB59385.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

“Programmed Death-1 (PD-1)” refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

“Programmed Death Ligand-1 (PD-L1)” is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

A “subject” includes any human or nonhuman animal. The term “nonhuman animal” includes, but is not limited to, vertebrates such as nonhuman primates, sheep, dogs, and rodents such as mice, rats and guinea pigs. In preferred embodiments, the subject is a human. The terms, “subject” and “patient” are used interchangeably herein.

The use of the term “flat dose” with regard to the methods and dosages of the invention means a dose that is administered to a patient without regard for the weight or body surface area (BSA) of the patient. The flat dose is therefore not provided as a mg/kg dose, but rather as an absolute amount of the agent (e.g., the anti-PD-1 antibody). For example, a 60 kg person and a 100 kg person would receive the same dose of an antibody (e.g., 240 mg of an anti-PD1 antibody).

The term “weight based dose” as referred to herein means that a dose that is administered to a patient is calculated based on the weight of the patient. For example, when a patient with 60 kg body weight requires 3 mg/kg of an anti-PD-1 antibody, one can calculate and use the appropriate amount of the anti-PD-1 antibody (i.e., 180 mg) for administration.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example, an “anti-cancer agent” promotes cancer regression in a subject. In preferred embodiments, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount of an anti-cancer agent preferably inhibits cell growth or tumor growth by at least about 20%, more preferably by at least about 40%, even more preferably by at least about 60%, and still more preferably by at least about 80% relative to untreated subjects. In other preferred embodiments of the invention, tumor regression may be observed and continue for a period of at least about 20 days, more preferably at least about 40 days, or even more preferably at least about 60 days. Notwithstanding these ultimate measurements of therapeutic effectiveness, evaluation of immunotherapeutic drugs must also make allowance for “immune-related” response patterns.

An “immune-related” response pattern refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents may require long-term monitoring of the effects of these agents on the target disease.

A therapeutically effective amount of a drug includes a “prophylactically effective amount,” which is any amount of the drug that, when administered alone or in combination with an anti-neoplastic agent to a subject at risk of developing a cancer (e.g., a subject having a pre-malignant condition) or of suffering a recurrence of cancer, inhibits the development or recurrence of the cancer. In preferred embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

A list of abbreviations is provided in Table 1.

TABLE 1
List of Abbreviations

| Term | Definition |
|---|---|
| AE | adverse event |
| ALT | alanine aminotransferase |
| ANC | absolute neutrophil count |
| ANC | absolute neutrophil count |
| AIDS | acquired immunodeficiency syndrome |
| aPTT | activated partial thromboplastin time |
| AE | AE |
| ALT | alanine aminotransferase |
| AST | aspartate aminotransferase |
| AT | aminotransaminases |
| AUC | area under the concentration-time curve |
| β-HCG | beta-human chorionic gonadotrophin |
| BID, bid | bis in die, twice daily |
| BICR | blinded independent central review |
| BMI | body mass index |
| BMS | Bristol-Myers Squibb |
| BP | blood pressure |
| BUN | blood urea nitrogen |
| C | Celsius |
| Ca++ | calcium |
| Cavg | average concentration |
| CBC | complete blood count |
| CFR | Code of Federal Regulations |
| CI | confidence interval |
| C1− | chloride |
| CLcr | creatinine clearance |
| Cm | centimeter |
| CNS | Central nervous system |
| CRC | Clinical Research Center |
| CRF | Case Report Form, paper or electronic |
| CTLA-4 | cytotoxic t lymphocyte-associated antigen 4 |
| CYP | cytochrome p-450 |
| D/C | discontinue |
| dL | deciliter |
| ECG | electrocardiogram |
| ECOG | Eastern Cooperative Oncology Group |
| eCRF | Electronic Case Report Form |
| EDC | Electronic Data Capture |
| EEG | electroencephalogram |
| Eg | exempli gratia (for example) |
| ESR | Expedited Safety Report |
| FDA | Food and Drug Administration |
| FISH | fluorescent in situ hybridization |
| FSH | follicle stimulating hormone |
| G | gram |
| GCP | Good Clinical Practice |
| GGT | gamma-glutamyl transferase |
| GFR | glomerular filtration rate |
| H | hour |
| HBsAg | hepatitis B surface antigen |
| HBV | hepatitis B virus |
| HCV | hepatitis C virus |
| HCO3− | bicarbonate |
| HIV | Human Immunodeficiency Virus |
| HR | heart rate |
| HRQol | health related quality of life |
| HRT | hormone replacement therapy |
| ICD | International Classification of Diseases |
| ICF | informed consent form |
| ICH | International Conference on Harmonisation |
| Ie | id est (that is) |
| IEC | Independent Ethics Committee |
| IMP | investigational medicinal products |
| IND | Investigational New Drug Exemption |
| IRB | Institutional Review Board |
| IU | International Unit |
| IU/L | international unit per liter |
| IU/mL | international unit per milliliter |
| IVRS | interactive voice response system |
| IV | intravenous |
| K3EDTA | potassium ethylenediaminetetraacetic acid |
| K+ | potassium |
| kD | kilodalton |
| Kg | kilogram |
| KM | kaplan-meier |
| L | liter |
| LCSS | lung cancer symptom scale |
| LDH | lactate dehydrogenase |
| mAB | monoclonal antibody |
| Mg | milligram |
| Mg++ | magnesium |
| MDSC | myeloid derived suppressor cells |
| Min | minute |
| mL | milliliter |
| mmHg | millimeters of mercury |
| MTD | maximum tolerated dose |
| mWHO | modified World Health Organization |
| μg | microgram |
| N | number of subjects or observations |
| Na+ | sodium |
| N/A | not applicable |
| NE | not evaluable |
| Ng | nanogram |
| NIMP | non-investigational medicinal products |
| NSAID | nonsteroidal anti-inflammatory drug |
| ORR | overall response rate |
| OS | overall survival |
| PD | pharmacodynamics |
| PD | progressive disease |
| PD-1 | programmed death-1 |
| PD-L1 | programmed death-ligand 1 |
| PD-L2 | programmed death-ligand 2 |
| PFS | progression-free survival |
| PR | partial response |
| PK | pharmacokinetics |
| PO | per os (by mouth route of administration) |
| PT | prothrombin time |
| PTT | partial thromboplastin time |
| QC | quality control |
| QD, qd | quaque die, once daily |
| RCC | renal cell carcinoma |
| RECIST 1.1 | response evaluation criteria in solid tumors version 1.1 |
| RBC | red blood cell |
| SAE | serious adverse event |
| SD | standard deviation |
| SD | stable disease |
| SOP | Standard Operating Procedures |
| Subj | subject |
| T | temperature |
| T | time |
| TAO | Trial Access Online, the BMS |
| | implementation of an EDC capability |
| T-HALF | Half life |
| TID, tid | ter in die, three times a day |
| TILs | tumor infiltrating lymphocytes |
| TSH | thyroid stimulating hormone |
| Tmax, TMAX | time of maximum observed concentration |
| ULN | upper limit of normal |
| VAS | visual analog scale |
| WBC | white blood cell |
| WHO | World Health Organization |
| WOCBP | women of childbearing potential |

Various aspects of the invention are described in further detail in the following subsections.

### Anti-PD-1 Antibodies

Human monoclonal antibodies that bind specifically to PD-1 with high affinity have been disclosed in U.S. Pat. No. 8,008,449. Other anti-PD-1 monoclonal antibodies have been described in, for example, U.S. Pat. Nos. 6,808,710, 7,488,802, 8,168,757 and 8,354,509, and PCT Publication No. WO 2012/145493. Each of the anti-PD-1 human monoclonal antibodies disclosed in U.S. Pat. No. 8,008,449 has been demonstrated to exhibit one or more of the following characteristics: (a) binds to human PD-1 with a K_{D} of 1×10⁻⁷ M or less, as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-γ production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates antibody responses; and (j) inhibits tumor cell growth in vivo. Anti-PD-1 antibodies usable in the present invention include monoclonal antibodies that bind specifically to human PD-1 and exhibit at least one, preferably at least five, of the preceding characteristics. A preferred anti-PD-1 antibody is nivolumab. Another preferred anti-PD-1 antibody is pembrolizumab.

Anti-PD-1 antibodies usable in the disclosed methods also include isolated antibodies that bind specifically to human PD-1 and cross-compete for binding to human PD-1 with nivolumab (see, e.g., U.S. Pat. No. 8,008,449; WO 2013/173223). The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of nivolumab by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., WO 2013/173223).

In certain embodiments, the antibodies that cross-compete for binding to human PD-1 with, or bind to the same epitope region of human PD-1 as, nivolumab are monoclonal antibodies. For administration to human subjects, these cross-competing antibodies are preferably chimeric antibodies, or more preferably humanized or human antibodies. Such chimeric, humanized or human monoclonal antibodies can be prepared and isolated by methods well known in the art.

Anti-PD-1 antibodies usable in the methods of the disclosed invention also include antigen-binding portions of the above antibodies. It has been amply demonstrated that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; and (iv) a Fv fragment consisting of the V_{L} and V_{H} domains of a single arm of an antibody.

### Anti-CTLA-4 Antibodies

Anti-CTLA-4 antibodies of the instant invention bind to human CTLA-4 so as to disrupt the interaction of CTLA-4 with a human B7 receptor. Because the interaction of CTLA-4 with B7 transduces a signal leading to inactivation of T-cells bearing the CTLA-4 receptor, disruption of the interaction effectively induces, enhances or prolongs the activation of such T cells, thereby inducing, enhancing or prolonging an immune response.

Human monoclonal antibodies that bind specifically to CTLA-4 with high affinity have been disclosed in U.S. Pat. Nos. 6,984,720 and 7,605,238. Other anti-PD-1 monoclonal antibodies have been described in, for example, U.S. Pat. Nos. 5,977,318, 6,051,227, 6,682,736, and 7,034,121. The anti-PD-1 human monoclonal antibodies disclosed in U.S. Pat. Nos. 6,984,720 and 7,605,238 have been demonstrated to exhibit one or more of the following characteristics: (a) binds specifically to human CTLA-4 with a binding affinity reflected by an equilibrium association constant (Kₐ) of at least about 10⁷M⁻¹, or about 10⁹M⁻¹, or about 10¹⁰ M⁻¹ to 10¹¹M⁻¹ or higher, as determined by Biacore analysis; (b) a kinetic association constant (kₐ) of at least about 10³, about 10⁴, or about 10⁵ m⁻¹ s⁻¹; (c) a kinetic disassociation constant (k_{d}) of at least about 10³, about 10⁴, or about 10⁵ m⁻¹ s⁻¹; and (d) inhibits the binding of CTLA-4 to B7-1 (CD80) and B7-2 (CD86). Anti-CTLA-4 antibodies usable in the present invention include monoclonal antibodies that bind specifically to human CTLA-4 and exhibit at least one, and preferably at least three of the preceding characteristics. An exemplary clinical anti-CTLA-4 antibody is the human monoclonal antibody 10D1 (now known as ipilimumab and marketed as YERVOY®) as disclosed in U.S. Pat. No. 6,984,720. Ipilimumab is a preferred anti-CTLA-4 antibody for use in the methods disclosed herein. Another anti-CTLA-4 antibody usable in the present methods is tremelimumab.

Anti-CTLA-4 antibodies usable in the disclosed methods also include isolated antibodies that bind specifically to human PD-1 and cross-compete for binding to human CTLA-4 with ipilimumab or tremelimumab or bind to the same epitope region of human CTLA-4 as ipilimumab or tremelimumab. In certain preferred embodiments, the antibodies that cross-compete for binding to human CTLA-4 with, or bind to the same epitope region of human PD-1 as does ipilimumab or tremelimumab, are antibodies comprising a heavy chain of the human IgG1 isotype. For administration to human subjects, these cross-competing antibodies are preferably chimeric antibodies, or more preferably humanized or human antibodies. Usable anti-CTLA-4 antibodies also include antigen-binding portions of the above antibodies such as Fab, F(ab′)₂, Fd, or Fv fragments.

### Standard-of-Care Therapies for Lung Cancer

Standard-of-care therapies for different types of cancer are well known by persons of skill in the art. For example, the National Comprehensive Cancer Network (NCCN), an alliance of 21 major cancer centers in the USA, publishes the NCCN Clinical Practice Guidelines in Oncology (NCCN GUIDELINES®) that provide detailed up-to-date information on the standard-of-care treatments for a wide variety of cancers (see NCCN GUIDELINES®, 2014).

NSCLC is the leading cause of cancer death in the U.S. and worldwide, exceeding breast, colon and prostate cancer combined. In the U.S., an estimated 228,190 new cases of lung and bronchial will be diagnosed in the U.S., and some 159,480 deaths will occur because of the disease (Siegel et al., 2013; Siegel et al. (2014) CA Cancer J Clin 64(1):9-29). The majority of patients (approximately 78%) are diagnosed with advanced/recurrent or metastatic disease. Metastases to the adrenal gland from lung cancer are a common occurrence, with about 33% of patients having such metastases. NSCLC therapies have incrementally improved OS, but benefit has reached a plateau (median OS for late stage patients is just 1 year). Progression after 1 L therapy occurred in nearly all of these subjects and the 5-year survival rate is only 3.6% in the refractory setting. From 2005 to 2009, the overall 5-year relative survival rate for lung cancer in the U.S. was 15.9% (NCCN GUIDELINES®, Version 3.2014—Non-Small Cell Lung Cancer, available at: www.nccn.org/professionals/physician_gls/pdf/nscl.pdf, last accessed May 14, 2014).

Surgery, RT and chemotherapy are the three modalities commonly used to treat NSCLC patients. As a class, NSCLCs are relatively insensitive to chemotherapy and RT, compared to small cell carcinoma. In general, for patients with Stage I or II disease, surgical resection provides the best chance for cure, with chemotherapy increasingly being used both pre-operatively and post-operatively. RT can also be used as adjuvant therapy for patients with resectable NSCLC, the primary local treatment, or as palliative therapy for patients with incurable NSCLC.

Patients with Stage IV disease who have a good performance status (PS) benefit from chemotherapy. Many drugs, including platinum agents (e.g., cisplatin, carboplatin), taxanes agents (e.g., paclitaxel, albumin-bound paclitaxel, docetaxel), vinorelbine, vinblastine, etoposide, pemetrexed and gemcitabine are useful for Stage IV NSCLC. Combinations using many of these drugs produce 1-year survival rates of 30% to 40% and are superior to single agents. Specific targeted therapies have also been developed for the treatment of advanced lung cancer. For example, bevacizumab (AVASTIN®) is a monoclonal antibody that blocks vascular endothelial growth factor A (VEGF-A). Erlotinib (TARCEVA®) is a small-molecule TKI of epidermal growth factor receptor (EGFR). Crizotinib (XALKORI®) is a small-molecule TKI that targets ALK and MET, and is used to treat NSCLC in patients carrying the mutated ALK fusion gene. Cetuximab (ERBITUX®) is a monoclonal antibody that targets EGFR.

There is a particular unmet need among patients who have squamous cell NSCLC (representing up to 25% of all NSCLC) as there are few treatment options after 1 L therapy. Single-agent chemotherapy is standard of care following progression with platinum-based doublet chemotherapy (Pt-doublet), resulting in median OS of approximately 7 months. Docetaxel remains the benchmark treatment in this line of therapy although erlotinib may also be used with less frequency. Pemetrexed has also been shown to produce clinically equivalent efficacy outcomes but with significantly fewer side effects compared with docetaxel in the 2 L treatment of patients with advanced NSCLC (Hanna et al., J Clin Oncol 22:1589-97 2004). No therapy is currently approved for use in lung cancer beyond the 3 L setting. Pemetrexed and bevacizumab are not approved in squamous NSCLC, and molecularly targeted therapies have limited application. The unmet need in advanced lung cancer has been compounded by the recent failure of Oncothyreon and Merck KgaA's STIMUVAX® to improve OS in a phase 3 trial, inability of ArQule's and Daiichi Sankyo's c-Met kinase inhibitor, tivantinib, to meet survival endpoints, failure of Eli Lilly's ALIMTA® in combination with Roche's AVASTIN® to improve OS in a late-stage study, and Amgen's and Takeda Pharmaceutical's failure to meet clinical endpoints with the small-molecule VEGF-R antagonist, motesanib, in late-stage trials.

### Immunotherapy of Lung Cancer

A clear need exists for effective agents for patients who have progressed on multiple lines of targeted therapy, as well as for therapies that extend survival for longer periods beyond the current standard treatments. Newer approaches involving immunotherapy, especially blockade of immune checkpoints including the CTLA-4, PD-1, and PD-L1 inhibitory pathways, have recently shown promise (Creelan et al., 2014). Thus, ipilimumab in combination with chemotherapy has exhibited encouraging results in small-cell and non-small-cell lung cancer alike. Clinical trials of the monoclonal antibodies nivolumab, pembrolizumab, BMS-936559, MEDI4736, and MPDL3280A are demonstrating durable overall radiological response rates in the 20% to 25% range in lung cancer (Topalian et al, 2012a; Pardoll, 2012; WO 2013/173223; Creelan et al., 2014). This exceptional activity includes squamous lung cancers, a population historically bereft of significant therapeutic advances.

In addition, dual checkpoint blockade strategies, such as those combining anti-PD-1 and anti-CTLA-4 have proven to be highly effective in treating melanoma (Wolchok et al, 2013; WO 2013/173223), and other combinations including anti-PD-L1, anti-LAG-3, or anti-KIR, are being tested to increase the proportion and durability of tumor responses. By analogy to melanoma, NSCLC patients may be able to benefit either from the combination of different immunotherapeutic drugs or the combination of such drugs with targeted agents or other treatments including, surgery, radiation, standard cancer chemotherapies, or vaccines. However, surprising and unexpected complications have sometimes been observed when immunotherapeutics are combined with other anti-cancer agents. Thus, the combination of immunotherapy (including an immune checkpoint inhibitor drug such as an anti-CTLA-4 or anti-PD-1 antibody) with other anti-cancer agents is unpredictable and must be carefully assessed for safety as well as efficacy in clinical trials. Although the combination of nivolumab and ipilimumab has proven to be very efficacious in treating melanoma with manageable toxicity (Wolchok et al., 2013), it was not hitherto known whether this combination would be significantly more effective in human subjects than treatment of NSCLC and other cancers with the individual agents.

### Pharmaceutical Compositions and Dosages

Therapeutic agents of the present invention may be constituted in a composition, e.g., a pharmaceutical composition containing an antibody or a TKI and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. Preferably, the carrier for a composition containing an antibody is suitable for intravenous, intramuscular, subcutaneous, parenteral, spinal or epidermal administration (e.g., by injection or infusion), whereas the carrier for a composition containing a TKI is suitable for non-parenteral, e.g., oral, administration. A pharmaceutical composition of the invention may include one or more pharmaceutically acceptable salts, anti-oxidant, aqueous and non-aqueous carriers, and/or adjuvants such as preservatives, wetting agents, emulsifying agents and dispersing agents.

Dosage regimens are adjusted to provide the optimum desired response, e.g., a maximal therapeutic response and/or minimal adverse effects. In some embodiments, the anti-PD-1 antibody is administered at a weight-based dose. For administration of an anti-PD-1 antibody, especially in combination with another anti-cancer agent, the dosage may range from about 0.01 to about 20 mg/kg, from about 0.1 to about 10 mg/kg, from about 0.01 to about 5 mg/kg, from about 1 to about 5 mg/kg, from about 2 to about 5 mg/kg, from about 1 to about 3 mg/kg, from about 7.5 to about 12.5 mg/kg, or from about 0.1 to about 30 mg/kg of the subject's body weight. For example, dosages can be about 0.1, about 0.3, about 1, about 2, about 3, about 5, or about 10 mg/kg body weight, and more preferably, 0.3, 1, 2, 3, or 5 mg/kg body weight. In certain embodiments, the dosage of the anti-PD-1 antibody is 3 mg/kg body weight.

In one embodiment, a dosage regimen for an anti-PD-1 antibody of the disclosure comprises about 0.3-1 mg/kg body weight, about 5 mg/kg body weight, 1-5 mg/kg body weight, or about 1-about 3 mg/kg body weight via intravenous administration, with the antibody being given every about 14-21 days in up to about 6-week or about 12-week cycles until complete response or confirmed progressive disease. In some embodiments, the antibody treatment, or any combination treatment disclosed herein, is continued for at least about 1 month, at least about 3 months, at least about 6 months, at least about 9 months, at least about 1 year, at least about 18 months, at least about 24 months, at least about 3 years, at least about 5 years, or at least about 10 years.

The dosing schedule is typically designed to achieve exposures that result in sustained receptor occupancy (RO) based on typical pharmacokinetic properties of an antibody. An exemplary treatment regime entails administration once per week, once every 2 weeks, once every 3 weeks, once every 4 weeks, once a month, once every 3-6 months or longer. In certain preferred embodiments, an anti-PD-1 antibody such as nivolumab is administered to the subject once every 2 weeks. In other preferred embodiments, the antibody is administered once every 3 weeks. The anti-PD-1 antibody can be administered in at least two doses, each of the doses is at an amount of about 0.01 mg/kg to about 5 mg/kg, e.g., 3 mg/kg, at a dosing interval of every two weeks between the two doses. In some embodiments, the anti-PD-1 antibody is administered in at least three, four, five, six, or seven doses (i.e., multiple doses), each of the doses is at an amount of about 0.01 mg/kg to about 5 mg/kg, e.g., 3 mg/kg, at a dosing interval of every two weeks between two adjacently given doses. The dosage and scheduling may change during a course of treatment. For example, a dosing schedule for anti-PD-1 monotherapy may comprise administering the antibody: (i) every 2 weeks in 6-week cycles; (ii) every 4 weeks for six dosages, then every three months; (iii) every 3 weeks; or (iv) 3-10 mg/kg once followed by 1 mg/kg every 2-3 weeks. Considering that an IgG4 antibody typically has a half-life of 2-3 weeks, a preferred dosage regimen for an anti-PD-1 antibody of the invention comprises 0.3-10 mg/kg body weight, preferably 1-5 mg/kg body weight, more preferably 1-3 mg/kg body weight via intravenous administration, with the antibody being given every 14-21 days in up to 6-week or 12-week cycles until complete response or confirmed progressive disease.

In certain embodiments, an anti-PD-1 antibody is administered at a flat dose. In embodiments, the anti-PD-1 antibody is administered at a flat dose as a monotherapy. In embodiments, the anti-PD-1 antibody is administered as a flat dose in combination with any other therapy disclosed herein. In embodiments, the flat dose of the anti-PD-1 antibody is a dose of at least about 100-600 mg, such as, at least about 200-300 mg, at least about 400-500 mg, or at least about 240 mg or at least about 480 mg, such as at least about 60 mg, at least about 80 mg, at least about 100 mg, at least about 120 mg, at least about 140 mg, at least about 160 mg, at least about 180 mg, at least about 200 mg, at least about 220 mg, at least about 240 mg, at least about 260 mg, at least about 280 mg, at least about 320 mg, at least about 360 mg, at least about 400 mg, at least about 440 mg, at least about 480 mg, at least about 520 mg, at least bout 560 mg, at least about 600 mg, or at least about 660 mg, or at least about 720 mg. In some embodiments, the anti-PD-1 antibody or antigen-binding portion thereof is administered at a dose of at least about 240 mg or at least about 480 mg once about every 2 or 4 weeks. In other embodiments, the anti-PD-1 antibody or antigen-binding portion thereof is administered at a dose higher than, i.e., at least about, 240 mg. When used in combinations with other cancer agents, the dosage of an anti-PD-1 antibody may be lowered compared to the monotherapy dose. For example, a dosage of nivolumab that is significantly lower than the typical 3 mg/kg every 3 weeks, for instance 0.1 mg/kg or less every 3 or 4 weeks, is regarded as a subtherapeutic dosage. Receptor-occupancy data from 15 subjects who received 0.3 mg/kg to 10 mg/kg dosing with nivolumab indicate that PD-1 occupancy appears to be dose-independent in this dose range. Across all doses, the mean occupancy rate was 85% (range, 70% to 97%), with a mean plateau occupancy of 72% (range, 59% to 81%) (Brahmer et al., J Clin Oncol 28:3167-75 2010). Thus, 0.3 mg/kg dosing may allow for sufficient exposure to lead to maximal biologic activity.

Although higher nivolumab monotherapy dosing up to 10 mg/kg every two weeks has been achieved without reaching the maximum tolerated does (MTD), the significant toxicities reported in other trials of checkpoint inhibitors plus anti-angiogenic therapy (see, e.g., Johnson et ah, 2013; Rini et al., 2011) support the selection of a nivolumab dose lower than 10 mg/kg.

Ipilimumab (YERVOY®) is approved for the treatment of melanoma at 3 mg/kg given intravenously once every 3 weeks for 4 doses. Thus, in preferred embodiments, 3 mg/kg is the highest dosage of ipilimumab used in combination with the anti-PD-1 antibody though, in certain embodiments, an anti-CTLA-4 antibody such as ipilimumab may be dosed within the range of about 0.3-10 mg/kg body weight every two or three weeks when combined with nivolumab. A dosage of ipilimumab that is significantly lower than the approved 3 mg/kg every 3 weeks, for instance 0.3 mg/kg or less every 3 or 4 weeks, is regarded as a subtherapeutic dosage. It has been shown that combination dosing of nivolumab at 3 mg/kg and ipilimumab at 3 mg/kg exceeded the MTD in a melanoma population, whereas a combination of nivolumab at 1 mg/kg plus ipilimumab at 3 mg/kg or nivolumab at 3 mg/kg plus ipilimumab at 1 mg/kg was found to be tolerable in melanoma patients (Wolchok et al., 2013). Accordingly, although nivolumab is tolerated up to 10 mg/kg given intravenously every 2 weeks, in preferred embodiments doses of the anti-PD-1 antibody do not exceed 3 mg/kg when combined with ipilimumab. In certain embodiments, based on risk-benefit and PK-PD assessments, the dosage used comprises a combination of nivolumab at 1 mg/kg plus ipilimumab at 3 mg/kg, nivolumab at 3 mg/kg plus ipilimumab at 1 mg/kg, or nivolumab at 3 mg/kg plus ipilimumab at 3 mg/kg is used, each administered at a dosing frequency of once every 2-4 weeks, preferably once every 3 weeks. In certain other embodiments, nivolumab is administered at a dosage of 0.1, 0.3, 1, 2, 3 or 5 mg/kg in combination with ipilimumab administered at a dosage of 0.1, 0.3, 1, 2, 3 or 5 mg/kg, once every 2 weeks, once every 3 weeks, or once every 4 weeks.

In certain embodiments, the combination of an anti-PD-1 antibody and an anti-CTLA-4 antibody is administered intravenously to the subject in an induction phase every 2 or 3 weeks for 2, 3 or 4 administrations. In certain preferred embodiments, the combination of nivolumab and ipilimumab is administered intravenously in the induction phase every 3 weeks for 4 administrations. The induction phase is followed by a maintenance phase during which only the anti-PD-1 antibody is administered to the subject at a dosage of 0.1, 0.3, 1, 2, 3, 5 or 10 mg/kg every two or three weeks for as long as the treatment proves efficacious or until unmanageable toxicity or disease progression occurs. In certain preferred embodiments, nivolumab is administered during the maintenance phase at a dose of 3 mg/kg body every 2 weeks.

For combination of nivolumab with other anti-cancer agents, these agents are preferably administered at their approved dosages. Treatment is continued as long as clinical benefit is observed or until unacceptable toxicity or disease progression occurs. Nevertheless, in certain embodiments, the dosages of these anti-cancer agents administered are significantly lower than the approved dosage, i.e., a subtherapeutic dosage, of the agent is administered in combination with the anti-PD-1 antibody. The anti-PD-1 antibody may be administered at the dosage that has been shown to produce the highest efficacy as monotherapy in clinical trials, e.g., about 3 mg/kg of nivolumab administered once every three weeks (Topalian et al., 2012a; Topalian et al., 2012), or at a significantly lower dose, i.e., at a subtherapeutic dose.

Dosage and frequency vary depending on the half-life of the antibody in the subject. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is typically administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present invention can be administered via one or more routes of administration using one or more of a variety of methods well known in the art. As will be appreciated by the skilled artisan, the route and/or mode of administration will vary depending upon the desired results.

### Methods of the Invention

This disclosure provides a method of treating a subject afflicted with a lung cancer, which method comprises administering to the subject a combination of therapeutically effective amounts of: (a) an anti-cancer agent which is an antibody or an antigen-binding portion thereof that specifically binds to and a PD-1 receptor and inhibits PD-1 activity; and (b) another anti-cancer agent. As NSCLC comprises more than 85% of lung tumors, in preferred embodiments the lung cancer is NSCLC. In other preferred embodiments, the subject is a human patient. In certain embodiments, the subject is a chemotherapy-naïve patient (e.g., a patient who has not previously received any chemotherapy). In other embodiments, the subject has received another cancer therapy (e.g., a chemotherapy), but is resistant or refractory to such another cancer therapy. In certain specific embodiments, the subject has cancer cells expressing mutated forms of the EGFR or KRAS gene.

In certain embodiments, the combination therapy of the present invention (e.g., administration of an anti-PD-1 antibody and another anti-cancer agent) effectively increases the duration of survival of the subject. For example, the duration of survival of the subject is increased by at least about 2 months when compared to another subject treated with only one therapy (e.g., an anti-PD-1 antibody or another anti-cancer agent). In certain embodiments, the combination therapy of the present invention (e.g., administration of an anti-PD-1 antibody and another anti-cancer agent) effectively increases the duration of progression free survival of the subject. For example, the progression free survival of the subject is increased by at least about 2 months when compared to another subject treated with only one therapy (e.g., an anti-PD-1 antibody or another anti-cancer agent). In certain embodiments, the combination therapy of the present invention (e.g., administration of an anti-PD-1 antibody and another anti-cancer agent) effectively increases the response rate in a group of subjects. For example, the response rate in a group of subjects is increased by at least 2% when compared to another group of subjects treated with only one therapy (e.g., an anti-PD-1 antibody or another anti-cancer agent).

### Anti-PD-1 and Anti-PD-L1 Antibodies Suitable for Use in the Disclosed Methods

Anti-PD-1 antibodies suitable for use in the disclosed methods are antibodies that bind to PD-1 with high specificity and affinity, block the binding of PD-L1 and or PD-L2, and inhibit the immunosuppressive effect of the PD-1 signaling pathway. In any of the therapeutic methods disclosed herein, an anti-PD-1 or anti-CTLA-4 “antibody” includes an antigen-binding portion or fragment that binds to the PD-1 or CTLA-4 receptor, respectively, and exhibits the functional properties similar to those of whole antibodies in inhibiting ligand binding and upregulating the immune system. In certain embodiments, the anti-PD-1 antibody or antigen-binding portion thereof cross-competes with nivolumab for binding to human PD-1. In other embodiments, the anti-PD-1 antibody or antigen-binding portion thereof is a chimeric, humanized or human monoclonal antibody or a portion thereof. In certain preferred embodiments for treating a human subject, the antibody is a humanized antibody. In other preferred embodiments for treating a human subject, the antibody is a human antibody. Antibodies of an IgG1, IgG2, IgG3 or IgG4 isotype may be used.

In certain embodiments, the anti-PD-1 antibody or antigen-binding portion thereof comprises a heavy chain constant region which is of a human IgG1 or IgG4 isotype. In certain other embodiments, the sequence of the IgG4 heavy chain constant region of the anti-PD-1 antibody or antigen-binding portion thereof contains an S228P mutation which replaces a serine residue in the hinge region with the proline residue normally found at the corresponding position in IgG1 isotype antibodies. This mutation, which is present in nivolumab, prevents Fab arm exchange with endogenous IgG4 antibodies, while retaining the low affinity for activating Fc receptors associated with wild-type IgG4 antibodies (Wang et al., 2014). In yet other embodiments, the antibody comprises a light chain constant region which is a human kappa or lambda constant region. In other embodiments, the anti-PD-1 antibody or antigen-binding portion thereof is a monoclonal antibody or an antigen-binding portion thereof. In certain preferred embodiments of any of the therapeutic methods described herein comprising administration of an anti-PD-1 antibody, the anti-PD-1 antibody is nivolumab. In other preferred embodiments, the anti-PD-1 antibody is pembrolizumab. In other embodiments, the anti-PD-1 antibody is chosen from the human antibodies 17D8, 2D3, 4H1, 4A11, 7D3 and 5F4 described in U.S. Pat. No. 8,008,449.

Because anti-PD-1 and anti-PD-L1 target the same signaling pathway and have been shown in clinical trials to exhibit similar levels of efficacy in a variety of cancers, including RCC (see Brahmer et al., 2012; Topalian et al., 2012a; WO 2013/173223), an anti-PD-L1 antibody may be substituted for the anti-PD-1 antibody in any of the therapeutic methods disclosed herein. In certain preferred embodiments, the anti-PD-L1 antibody is BMS-936559 (formerly 12A4 or MDX-1105) (see, e.g., U.S. Pat. No. 7,943,743; WO 2013/173223). In other preferred embodiments, the anti-PD-L1 antibody is MPDL3280A (also known as RG7446) (see, e.g., Herbst et al. 2013; U.S. Pat. No. 8,217,149) or MEDI4736 (Khleif, 2013).

Combination of an Anti-PD-1 Antibody with an Anti-CTLA-4 Antibody for Treating NSCLC

This disclosure also provides combination therapy methods for treating NSCLC wherein an anti-PD-1 antibody is combined with another anti-cancer agent which is an antibody or an antigen-binding portion thereof that binds specifically to CTLA-4 and inhibits CTLA-4 activity. The combination of the anti-PD-1 antibody, nivolumab, and the anti-CTLA-4 antibody, ipilimumab has been demonstrated herein (see Example 4) to produce early, durable antitumor activity in NSCLC patients. Accordingly, in certain preferred embodiments, the anti-CTLA-4 antibody that is used in combination with the anti-PD-1 antibody is ipilimumab. In preferred embodiments, the anti-CTLA-4 antibody is tremelimumab. In other embodiments, the anti-CTLA-4 antibody or antigen-binding portion thereof is an antibody or antigen-binding portion thereof that cross-competes with ipilimumab for binding to human CTLA-4. In certain other embodiments, the anti-CTLA-4 antibody or antigen-binding portion thereof is a chimeric, humanized or human monoclonal antibody or a portion thereof. In yet other embodiments, the anti-CTLA-4 antibody or antigen-binding portion thereof comprises a heavy chain constant region which is of a human IgG1 or IgG4 isotype. In preferred embodiments, the anti-CTLA-4 antibody comprises a heavy chain constant region which is of a human IgG1 isotype.

For the combination of an anti-PD-1 and an anti-CTLA-4 antibody, the dosing regimen comprises an induction period (also referred to herein as an induction phase) during which one or more, preferably about four, combination doses of the anti-PD-1 and anti-CTLA-4 antibodies are administered to the patient, followed by a maintenance period or phase comprising dosing with the anti-PD-1 antibody alone, i.e., not including the anti-CTLA-4 antibody. In certain embodiments, the method comprises (a) an induction phase, wherein the anti-PD-1 and anti-CTLA-4 antibodies or antigen-binding portions thereof are administered in combination in 2, 4, 6, 8 or 10 doses, each dose ranging from 0.1 to 10.0 mg/kg body weight administered at least once every 2 weeks, once every 3 weeks, or once every 4 weeks, followed by (b) a maintenance phase, wherein no anti-CTLA-4 antibody or antigen-binding portion thereof is administered and the anti-PD-1 antibody or antigen-binding portion thereof is repeatedly administered at a dose ranging from 0.1 to 10 mg/kg at least once every 2 weeks, once every 3 weeks, or once every 4 weeks.

In certain embodiments, (a) the induction phase comprises at least 4 doses administered at 3-week intervals, wherein the anti-PD-1 and anti-CTLA-4 antibodies are administered at the following dosages: (i) 0.1 mg/kg anti-PD-1 antibody and 3 mg/kg of anti-CTLA-4 antibody; (ii) 0.3 mg/kg anti-PD-1 antibody and 3 mg/kg of anti-CTLA-4 antibody; (iii) 1 mg/kg anti-PD-1 antibody and 3 mg/kg of anti-CTLA-4 antibody; (iv) 3 mg/kg anti-PD-1 antibody and 3 mg/kg of anti-CTLA-4 antibody; (v) 5 mg/kg anti-PD-1 antibody and 3 mg/kg of anti-CTLA-4 antibody; (vi) 10 mg/kg anti-PD-1 antibody and 3 mg/kg of anti-CTLA-4 antibody; (vii) 0.1 mg/kg anti-PD-1 antibody and 1 mg/kg of anti-CTLA-4 antibody; (viii) 0.3 mg/kg anti-PD-1 antibody and 1 mg/kg of anti-CTLA-4 antibody; (ix) 1 mg/kg anti-PD-1 antibody and 1 mg/kg of anti-CTLA-4 antibody; (x) 3 mg/kg anti-PD-1 antibody and 1 mg/kg of anti-CTLA-4 antibody; (xi) 5 mg/kg anti-PD-1 antibody and 1 mg/kg of anti-CTLA-4 antibody; or (xii) 10 mg/kg anti-PD-1 antibody and 1 mg/kg of anti-CTLA-4 antibody, and (b) the maintenance phase comprises repeated administration of the anti-PD-1 antibody at a dose of 3 mg/kg every 2 weeks.

Because of durability of the clinical effect previously demonstrated with immunotherapy by inhibition of immune checkpoints (see, e.g., WO 2013/173223), the maintenance phase may include, in alternative embodiments, a finite number of doses, e.g., 1-10 doses, or may involve dosing at long intervals, e.g., once every 3-6 months or once every 1-2 years or longer intervals. The maintenance phase may be continued for as long as clinical benefit is observed or until unmanageable toxicity or disease progression occurs.

Given the uncertainty of whether the ipilimumab administered past week 12 contributes to clinical benefit in melanoma and the fact that the U.S. Food and Drug Administration (FDA)- and European Medicines Agency (EMA)-approved schedule for YERVOY® is every 3 weeks for a total of 4 doses, in preferred embodiments the anti-CTLA-4 antibody is administered during the induction phase once every 3 weeks for a total of 4 doses. Accordingly, in certain preferred embodiments, the method comprises (a) an induction phase consisting of 4 combination doses administered at 3-week intervals, wherein (i) the anti-PD-1 antibody or antigen-binding portion thereof is administered at 3 mg/kg body weight and the anti-CTLA-4 antibody or antigen-binding portion thereof is administered at 1 mg/kg body weight; (ii) the anti-PD-1 antibody or antigen-binding portion thereof is administered at 1 mg/kg body weight and the anti-CTLA-4 antibody or antigen-binding portion thereof is administered at 3 mg/kg body weight; (iii) the anti-PD-1 antibody or antigen-binding portion thereof is administered at 1 mg/kg body weight and the anti-CTLA-4 antibody or antigen-binding portion thereof is administered at 1 mg/kg body weight; or (iv) the anti-PD-1 antibody or antigen-binding portion thereof is administered at 3 mg/kg body weight and the anti-CTLA-4 antibody or antigen-binding portion thereof is administered at 3 mg/kg body weight; and (b) the maintenance phase comprises repeated administration of the anti-PD-1 antibody or antigen-binding portion thereof at a dose of 3 mg/kg every 2 weeks. In further embodiments of these methods, the maintenance phase is continued for as long as clinical benefit is observed or until unacceptable or unmanageable toxicity or disease progression occurs.

In certain preferred embodiments of the present methods, the anti-PD-1 antibody is nivolumab. In other preferred embodiments, it is pembrolizumab. In yet other preferred embodiments, the anti-CTLA-4 antibody is ipilimumab. In further embodiments, the anti-CTLA-4 antibody is tremelimumab. Typically, the anti-PD-1 and anti-CTLA-4 antibodies are formulated for intravenous administration. In certain embodiments, when the anti-PD-1 and anti-CTLA-4 antibodies are administered in combination, they are administered within 30 minutes of each other. Either antibody may be administered first, that is, in certain embodiments, the anti-PD-1 antibody is administered before the anti-CTLA-4 antibody, whereas in other embodiments, the anti-CTLA-4 antibody is administered before the anti-PD-1 antibody. In some embodiments, each antibody is administered by intravenous infusion over a period of 60 minutes or 30 minutes. In some embodiments, each antibody is administered by intravenous infusion over a period of less than 90 or less than 60 minutes, e.g., over a period of about 15-59 minutes, about 15-55 minutes, about 15-50 minutes, about 15-45 minutes, about 15-40 minutes, about 15-35 minutes, about 15-30 minutes, about 20-35 minutes, or about 20-30 minutes. In some embodiments, nivolumab is administered by intravenous infusion over a period of less than 60 minutes, e.g., over a period of about 15-59 minutes, about 15-55 minutes, about 15-50 minutes, about 15-45 minutes, about 15-40 minutes, about 15-35 minutes, about 15-30 minutes, about 20-35 minutes, or about 20-30 minutes. In some embodiments, ipilimumab is administered by intravenous infusion over a period of less than 90 or less than 60 minutes, e.g., over a period of about 15-59 minutes, about 15-55 minutes, about 15-50 minutes, about 15-45 minutes, about 15-40 minutes, about 15-35 minutes, about 15-30 minutes, about 20-35 minutes, or about 20-30 minutes. In certain embodiments, the anti-PD-1 and anti-CTLA-4 antibodies are administered concurrently, either admixed as a single composition in a pharmaceutically acceptable formulation for concurrent administration, or concurrently as separate compositions with each antibody in a pharmaceutically acceptable formulation.

Certain preferred embodiments of the present methods comprise (a) an induction phase consisting of administration of nivolumab by intravenous infusion followed by administration of ipilimumab by intravenous infusion every 3 weeks for 4 combination doses, followed by (b) maintenance dosing with nivolumab administered by intravenous infusion every 2 weeks starting 3 weeks after the 4th dose of induction therapy or after Day 113 of the 4th dose of induction therapy has not been administered due to treatment delays.

In certain embodiments, the anti-PD-1 antibody or antigen-binding portion thereof is administered at a subtherapeutic dose. In certain other embodiments, the anti-CTLA-4 antibody or antigen-binding portion thereof is administered at a subtherapeutic dose. In further embodiments, both the anti-PD-1 antibody or antigen-binding portion thereof and the anti-CTLA-4 antibody or antigen-binding portion thereof are each administered at a subtherapeutic dose.

In certain embodiments, the anti-PD-1 antibody or antigen-binding portion thereof is administered in a flat dose, e.g., at least about 240 mg or at least about 480 mg, every 2 weeks or every 4 weeks, in combination with another anti-cancer agent, e.g., an antibody or antigen-binding portion thereof that binds specifically to CTLA-4 and inhibits CTLA-4 activity (“an anti-CTLA-4 antibody or antigen-binding portion thereof”). In some embodiments, the ratio is at least about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1 mg anti-PD-1 antibody to mg anti-CTLA-4 antibody.

### Kits

Also within the scope of the present invention are kits comprising an anti-PD-1 antibody and another anti-cancer agent for therapeutic uses. Kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a lung cancer, the kit comprising: (a) a dosage ranging from 0.1 to 10 mg/kg body weight of an anti-cancer agent which is an antibody or an antigen-binding portion thereof that specifically binds to the PD-1 receptor and inhibits PD-1 activity; (b) a dosage of another anti-cancer agent which is a dosage ranging from 0.1 to 10 mg/kg body weight of an antibody or an antigen-binding portion thereof that specifically binds to and inhibits CTLA-4; and (c) instructions for using the anti-PD-1 antibody and the other anti-cancer agent in any of the combination therapy methods disclosed herein. In certain embodiments, the anti-PD-1, the anti-CTLA-4 antibody and/or the TKI may be co-packaged in unit dosage form. In certain preferred embodiments for treating human patients, the kit comprises an anti-human PD-1 antibody disclosed herein, e.g., nivolumab or pembrolizumab. In other preferred embodiments, the kit comprises an anti-human CTLA-4 antibody disclosed herein, e.g., ipilimumab or tremelimumab.

### Clinical Protocol CA209227

A phase 3 Open-Label, Randomized Phase 3 Trial of Nivolumab versus platinum doublet chemotherapy and Nivolumab plus Ipilimumab versus platinum doublet chemotherapy in Subjects with Chemotherapy-Naïve Stage IV or Recurrent Non-Small Cell Lung Cancer (NSCLC) is described in detail herein.

The tested products include: 1) nivolumab (BMS-936558) monotherapy administered IV over 30 minutes at 240 mg every 2 weeks until progression, unacceptable toxicity, or other reasons specified in the protocol, or 2) nivolumab administered IV over 30 minutes at 1 mg/kg combined with ipilimumab administered IV over 30 minutes at 1 mg/kg every 3 weeks for 4 doses, followed by nivolumab administered IV over 30 minutes at 3 mg/kg every 2 weeks until progression, unacceptable toxicity, or other reasons specified in the protocol, or 3) nivolumab administered IV over 30 minutes at 3 mg/kg every 2 weeks combined with ipilimumab administered IV over 30 minutes at 1 mg/kg every 6 weeks until progression, unacceptable toxicity, or other reasons specified in the protocol, or 4) platinum doublet chemotherapy, based on tumor histology, for up to 6 doses as follows: subjects with squamous histology may receive either gemcitabine (1250 mg/m²) with cisplatin (75 mg/m²) or gemcitabine (1000 mg/m²) with carboplatin (AUC 5); subjects with non-squamous histology may receive pemetrexed (500 mg/m²) with either cisplatin (75 mg/m²) or carboplatin (AUC 6); subjects with non-squamous histology may also receive optional continuation maintenance therapy with pemetrexed alone until disease progression or unacceptable toxicity.

The study includes both Investigational [Medicinal] Products (IP/IMP) and Non-investigational [Medicinal] Products (Non-IP/Non-IMP) as listed in Table 2.

TABLE 2
Study Drugs for CA209227

| Medication | Potency | | IP/Non-IP |
|---|---|---|---|
| Nivolumab | 10 | mg/ml | IP |
| Ipilimumab | 5 | mg/ml | IP |
| Carboplatin | 10 | mg/ml | IP |
| Cisplatin | 1 | mg/ml | IP |
| Gemcitabine | 1000 | mg/vial | IP |
| Pemetrexed | 500 | mg/vial | IP |

The study assessment includes, e.g., overall survival (OS) as a primary endpoint. Overall survival is defined as the time from randomization to the date of death. Subjects will be assessed for response by CT or MRI beginning at 6 weeks (±7 days) after randomization and continuing every 6 weeks (±7 days) until week 48 and then every 12 weeks (±7 days) until progression or treatment discontinuation, whichever occurs later. Tumor assessments continue per protocol until RECIST 1.1 progression is assessed. A subject who has not died will be censored at last known alive date. OS will be followed continuously while subjects are on the study drugs and every 3 months. All randomized subjects are evaluated.

### Study Considerations

Non-small cell lung cancer (NSCLC) remains the leading cause of cancer-related mortality worldwide, accounting for approximately 18% of all cancer deaths. (Jemal A, et al. Global Cancer Statistics CA Cancer J Clin 2011; 61:69-90.) Despite treatment with platinum-based chemotherapy, the standard of care for first-line therapy patients with metastatic NSCLC have a median survival of approximately 10 months, and a 5-year survival rate of less than 5%. (NCCN Clinical Practice Guidelines in Oncology. Non-small cell lung cancer. v.3.2014. www.nccn.org.)

Immunotherapeutic approaches recently have demonstrated clinical efficacy in several cancer types, including melanoma and hormone-refractory prostate cancer. (Mellman et al. Nature 2011 Dec. 22; 29 4 8 0:480-89.) Tumors may modulate and evade the host immune response through a number of mechanisms, including down regulation of tumor-specific antigen expression and presentation, secretion of anti-inflammatory cytokines, and upregulation of inhibitory ligands. T cell checkpoint regulators such as CTLA-4 and programmed death-1 (PD-1, CD279) are cell surface molecules that, when engaged by their cognate ligands, induce signaling cascades down-regulating T cell activation and proliferation. One proposed model by which therapeutic T cell checkpoint inhibitors derive antitumor activity is through breaking of immune tolerance to tumor cell antigens.

Nivolumab (BMS-936558) is a fully human, IgG4 (kappa) isotype monoclonal antibody that binds PD-1 on activated immune cells and disrupts engagement of the receptor with its ligands PD-L1 (B7-H1/CD274) and PD-L2 (B7-DC/CD273), thereby abrogating inhibitory signals and augmenting the host antitumor response. In early clinical trials, nivolumab has demonstrated activity in several tumor types, including melanoma, renal cell cancer (RCC), and NSCLC. (Brahmer et al. J Clin Oncol 2010; 28:3167-3175.) In particular, substantial activity has been noted in previously treated NSCLC subjects, where objective response rates approached 25%, and the progression-free survival (PFS) rate at 24 weeks approached 45%, with no clear differences between squamous and non-squamous histologies. (Nivolumab (BMS-936558) Investigator Brochure, version 13, 2014.)

Nivolumab (Opdivo®) was recently approved to treat patients with metastatic squamous cell NSCLC with progression on or after platinum-based chemotherapy. (Opdivo Prescribing Information, 2015.) The approval was based on the results of CA209017, a randomized trial of 272 patients, of whom 135 received nivolumab and 137 received docetaxel. The median overall survival (OS) for patients in the nivolumab arm was 9.6 months versus 6 months for those in the docetaxel arm (HR=0.59). A single arm trial (CA209063) of 117 patients with metastatic squamous cell NSCLC, with progression after platinum-based chemotherapy and at least one additional systemic regimen, showed a 15% overall response rate (ORR), of whom 59% had response durations of 6 months or longer. (Rizvi et al. Lancet Oncol 2015; Published online Feb. 20, 2015.)

In general, nivolumab also has been well tolerated to date, with a favorable safety profile relative to anticipated toxicities based on an immunostimulatory mechanism of action. (Amos et al. Autoimmunity associated with immunotherapy of cancer. Blood 2011; 118:499-509.)

Combining immunotherapeutic agents with different mechanisms of action offers the possibility of a synergistic response. PD-1 and CTLA-4 are both co-inhibitory molecules, but evidence suggests that they use distinct mechanisms to limit T cell activation. Preliminary indirect data from peripheral T cell assessments suggest that a given T-cell checkpoint inhibitor may modulate host immune cell phenotype rendering them more susceptible to alternate checkpoint inhibitors and thereby enhancing anti-tumor activity.

In a phase 1 study of the combination of nivolumab plus ipilimumab in advanced melanoma (CA209004), there was a 41% response rate, including a 17% complete response rate (CR). In a phase 1 study in patients with NSCLC (CA209012), the combination of nivolumab plus ipilimumab combination is being evaluated. Efficacy data appears to be comparable to or better than that observed for nivolumab monotherapy.

### Nivolumab Monotherapy (Arm A)

PD-1 is a 55 kD type I transmembrane protein primarily expressed on activated T cells, B cells, myeloid cells, and antigen-presenting cells (APCs). (Keir M E, et al. Annu. Rev. Immunol. 2008; 26:677-704.) Binding of PD-1 to PD-L1 and PD-L2 has been shown to down-regulate T-cell activation in both murine and human systems. (Freeman G J, et al. J Exp Med. 2000; 192:1027-34; Latchmanet al. Nat Immunol. 2001; 2:261-8; Carteret al. Eur J Immunol. 2002; 32:634-43; and Barber et al. Nature 2006; 439:682-7.) In particular, PD-L1 has been shown to be upregulated on several cancers types including NSCLC and, in some cases, correlated to negative prognosis. (Dong H, Chen L. J Mol Med. 2003; 81:281-7; Konishi et al. Clin Cancer Res. 2004; 10:5094-10; Thompson et al. Proc Natl Acad Sci USA. 2004; 101:17174-9; Mu et al. Med Oncol. 2011; 28:682-688; and Hamanishi et al. PNAS 2007; 104:3360-65.) PD-1/PD-L1 interactions may also indirectly modulate the response to tumor antigens through T-cell/APC interactions.

Therefore, PD-1 engagement may represent one means by which tumors evade immunosurveillance and clearance. (Pardoll D M. Nature 2012; 12:252-64.) Blockade of the PD-1 pathway by nivolumab has been studied in a variety of preclinical in vitro assays, and antitumor activity using a murine analog of nivolumab has been shown in a number of immunocompetent mouse cancer models. (Nivolumab (BMS-936558) Investigator Brochure, version 12, 2013.) Based on these and other preclinical data, PD-1 blockade by nivolumab has been pursued as a promising therapeutic strategy to reverse immune tolerance and enhance T-cell effector function in several tumor types including NSCLC.

Substantial monotherapy clinical activity has been observed in greater than or equal to second line NSCLC subjects (n=129) treated in the ongoing Phase 1 multi-dose, dose escalation study of nivolumab (CA209003). (Nivolumab (BMS-936558) Investigator Brochure, version 12, 2013.) This study showed objective response rates (ORR) of 22% (squamous) to 26% (non-squamous) at the 3 mg/kg dose, greater than the historical ORR for second-line docetaxel (approximately 8-10%) (Shepherd et al. J Clin Oncol. 2000; 18:2095-2103; Fossella et al. J Clin Oncol. 2000; 18:2354-62; Hanna et al. J Clin Oncol. 2004; 22:1589-97) and similar to that for first-line platinum doublet chemotherapy (approximately 25-30%) (Belani et al. J Clin Oncol 2008; 26: 468-73). In addition, the 24-week PFS rate among NSCLC subjects treated at the 3 mg/kg dose was 42% (non-squamous) to 45% (squamous). (Nivolumab (BMS-936558) Investigator Brochure, version 12, 2013.) By comparison, the historical median PFS for second-line docetaxel is approximately 3 months (Fossella et al. J Clin Oncol. 2000; 18:2354-62; Hanna et al. J Clin Oncol. 2004; 22:1589-97) and that for first-line platinum doublet chemotherapy is approximately 4 to 5.5 months (Belani et al. J Clin Oncol 2008; 26: 468-73; Scagliotti et al. J Clin Oncol 2008; 26: 3543-51). Among all NSCLC subjects with a response, the median duration of response was 74 weeks. Furthermore, the adverse event profile for nivolumab appears favorable versus platinum doublet chemotherapy as hematologic toxicities are currently rare, and the majority of non-hematologic toxicities are low grade and manageable. As previously noted, CA209063, a single arm study of 117 patients with metastatic squamous cell NSCLC with progression after platinum-based chemotherapy and at least one additional systemic regimen, showed an ORR of 15%, with 59% of response durations lasting 6 months or longer. (Rizvi et al. Lancet Oncol 2015; Published online Feb. 20, 2015.) CA209017 was a randomized trial of 272 patients; 135 were randomized to nivolumab and 137 to docetaxel. The median overall survival (OS) for patients in the nivolumab arm was 9.6 months versus 6 months for those in the docetaxel arm. Fifty-seven percent (74/129) of patients in the docetaxel arm experienced a Grade 3-5 treatment-related event, including 3 deaths, compared to 9 (6.9%) patients in the nivolumab arm, none of whom had a grade 5 event.

Nivolumab monotherapy at 3 mg/kg every 2 weeks has been evaluated in one of several cohorts of chemotherapy-naive patients with advanced NSCLC in study CA209012. Results from this cohort (n=52), including 13 subjects with squamous histology and 39 subjects with non-squamous histology, show an overall response rate (ORR) of 23% and a disease control rate (DCR) of 50%. The PFS rate at 24 weeks is 41%, with a median PFS of 16 weeks. The OS rate at 12 months is 74%, and the median overall survival (OS) is 22.6 months. The majority of responses have been durable, with the median duration of response not reached. In contrast, the median OS for patients with non-squamous histology who receive platinum doublet chemotherapy is 13 months, and for those with squamous histology is 10 months. (Ellis et al. J Clin Oncol (2014) 32:1277-1280.)

### Flat Dosing of Nivolumab Monotherapy

Nivolumab monotherapy has been studied in NSCLC patient population in studies CA209003, CA209063, CA209017, and CA209057 with body weight normalized dosing (mg/kg). Nivolumab pharmacokinetics (PK) and exposures of subjects in these studies have been characterized by population pharmacokinetic (PPK) analysis of data collected these studies, together with PK data from several phase 1, 2, and 3 clinical studies of nivolumab monotherapy in solid tumors. Nivolumab PK was determined to be linear, with dose proportional exposures over a dose range of 0.1 to 10 mg/kg. Nivolumab clearance and volume of distribution was found to increase with increasing body weight, but the increase was less than proportional, indicating that a mg/kg dose represents an over-adjustment for the effect of body weight on nivolumab PK. Conversely, given the relationship between nivolumab PK and body weight, a flat dose is expected to lead to lower exposures in heavier patients, relative to the exposures in lighter patients.

Table 3 presents summary statistics of the estimated nivolumab steady-state trough, peak and time-averaged concentration (Cminss, Cmaxss, and Cavgss, respectively) in NSCLC subjects receiving 3 mg/kg, together with corresponding statistics of exposures predicted for a flat nivolumab dose of 240 mg. It should be noted that a dose of 240 mg nivolumab is identical to a dose of 3 mg/kg for subjects weighing 80 kg, which is the approximate median body weight of NSCLC subjects in the 3 phase 2 and 3 clinical studies of nivolumab monotherapy in NSCLC patients (CA209017, CA209057, and CA209063). As evident from the data presented in Table 3, the geometric mean values of Cminss, Cmaxss, and Cavgss with flat dosing are slightly (<15%) higher than that produced by a 3 mg/kg dose, and the coefficient of variation (cv %) in these measures of exposure are only slightly (<10%) greater than that of 3 mg/kg dosing.

TABLE 3
Summary Statistics of Nivolumab Steady-state Exposure

| Nivolumab | Cminss Geo. | Cmaxss Geo. | Cavgss Geo. |
|---|---|---|---|
| Dose | Mean | Mean | Mean |
| 240 | 61.5 (44.6) | 133.7 (35.0) | 82.4 (38.2) |
| 3 | 54.7 (41.9) | 118.9 (31.8) | 73.3 (35.6) |

Nivolumab has been shown to be safe and well tolerated up to a dose level of 10 mg/kg, and the relationship between nivolumab exposure produced by 3 mg/kg and efficacy has been found to be relatively flat. Taken together, the PK, safety, and efficacy data indicate that the safety and efficacy profile of 240 mg nivolumab will be similar to that of 3 mg/kg nivolumab.

The PK and safety of nivolumab have been evaluated in the Asian population. The comparison of PK parameters in global and Japanese subjects suggests that the PK of nivolumab is similar in these populations. Nivolumab is shown to be safe and well tolerated in Japanese subjects. The similar PK and safety profile of nivolumab between global and Japanese subjects supports the use of similar dosing in the Asian population as is being used in global clinical studies.

One of the experimental arms in CA209227 will be nivolumab monotherapy 240 mg every 2 weeks for subjects with previously untreated stage IV or recurrent NSCLC.

Nivolumab in Combination with Ipilimumab (Arms B and C)

Preclinical data indicate that the combination of PD-1 and CTLA-4 receptor blockade may improve antitumor activity. In vitro combinations of nivolumab plus ipilimumab increase IFN-γ production 2- to 7-fold over either agent alone in a mixed lymphocyte reaction. Increased antitumor activity of the combination was also observed in 3 of 5 syngeneic murine cancer models. In a murine melanoma vaccine model, blockade with either CTLA-4 or PD-1 antibodies increased the proportion of CTLA-4 and PD-1-expressing CD4/CD8 tumor infiltrating T effector cells, and dual blockade increased tumor infiltration of T effector cells and decreased intratumoral T regulatory cells, as compared to either agent alone. (Curran et al. PNAS 2010; 107: 4275-80.)

Clinically, ipilimumab has been shown to have activity in lung cancer. A Phase 2 study (CA184041) in subjects with NSCLC or small cell lung cancer (SCLC) investigated the addition of ipilimumab to carboplatin and paclitaxel using 2 different schedules (concurrent and phased). The phased schedule demonstrated a significant improvement of immune-related progression-free survival (irPFS) activity compared to chemotherapy alone, in both NSCLC and SCLC. (Ipilimumab (BMS-734016) Investigator Brochure, version 17, 2014.)

The combination of nivolumab and ipilimumab is currently being evaluated in CA209004 (MDX1106-04), a Phase 1b multiple ascending dose study in subjects with treatment-naive and previously treated advanced melanoma. Both nivolumab and ipilimumab are given concurrently on an every 3 week schedule for 4 doses, followed by nivolumab alone every 3 weeks for 4 doses. A maintenance period was also evaluated in which the combined treatment is administered every 12 weeks for up to 8 doses. Treatment cohorts were as follows: cohort 1 (n=14): nivolumab 0.3 mg/kg+ipilimumab 3 mg/kg; cohort 2 (n=17): nivolumab 1 mg/kg+ipilimumab 3 mg/kg; cohort 2a (n=16): nivolumab 3 mg/kg+ipilimumab 1 mg/kg; and cohort 3 (n=6): nivolumab 3 mg/kg+ipilimumab 3 mg/kg.

Efficacy and safety results have been reported. (Wolchok et al. New Engl J Med 2013; 369: 122-33.) The data show that in cohort 3, DLTs were observed in 3 of 6 subjects (Grade 3/4 amylase/lipase for >3 weeks). The doses in cohort 2 were identified as the maximum doses that were associated with an acceptable level of adverse events (Grade 3 uveitis in one subject and Grade 3 elevated AST and ALT in one subject). Of the 52 subjects evaluable for response, 21 subjects (40%) had a confirmed objective response by modified World Health Organization (mWHO) criteria. An additional 4 subjects (8%) had an objective response according to immune-related criteria. A total of 16 subjects (31%) had tumor reduction of 80% or more at 12 weeks, including 5 with a complete response. Responses were ongoing in 19 or 21 subjects who had a response, with the duration ranging from 6.1 to 72.1 weeks at the time of data analysis. These results from CA209004 suggest that the combination of nivolumab and ipilimumab may have greater clinical activity than either agent alone in advanced melanoma, although with a potential for increased toxicity.

The nivolumab plus ipilimumab combination has been also evaluated at several different doses and schedules as first line therapy in patients with advanced NSCLC in CA209012, an ongoing Phase 1 study of nivolumab as monotherapy and in combination with a variety of agents. Early cohorts evaluated 2 different dosing schedules: nivolumab 1 mg/kg+ipilimumab 3 mg/kg, every 3 weeks for four cycles as induction, followed by nivolumab 3 mg/kg every 2 weeks; or nivolumab 3 mg/kg+ipilimumab 1 mg/kg, every 3 weeks for four cycles as induction, followed by nivolumab 3 mg/kg every 2 weeks.

These regimens resulted in significant toxicity, with 37% of patients discontinuing treatment due to a treatment-related adverse event. Thus, a third combination cohort was initiated: nivolumab 1 mg/kg+ipilimumab 1 mg/kg every 3 weeks (“1+1”) for 4 cycles as induction, followed by nivolumab 3 mg/kg every 2 weeks as maintenance. This dosing regimen has been much better tolerated with only 13% of subjects experiencing treatment-related AEs leading to discontinuation. The efficacy data appears to be comparable to or better than that observed for nivolumab monotherapy. The ORR is 16%, and the DCR is 58%. The PFS rate at 24 weeks is 55% with a median PFS of 46.1 weeks. The OS rate at 12 months is 63%, and the median OS is not reached. Activity has been observed in patients with both PD-L1 positive (PD-L1+) and PD-L1 negative (PD-L1−) tumors.

Currently, CA209012 is enrolling three additional cohorts of the nivolumab/ipilimumab combination. These are designed to test the hypothesis that safety may be improved by decreasing the dose and frequency of ipilimumab, and efficacy could be increased by dosing nivolumab every 2 weeks, allowing nivolumab to serve as the “base” of the combination. Preliminary data confirm an improved safety profile compared to the early cohorts, with discontinuation of treatment due to treatment-related AEs in the newer cohorts ranging from 5-11%. Response rates range from 15-30%, and disease control rates range from 38-51%.

CA209227 will have two nivolumab+ipilimumab treatment arms. One will evaluate the “1+1” schedule; the other arm will evaluate nivolumab 3 mg/kg every 2 weeks+ipilimumab 1 mg/kg, every 6 weeks until progression or unacceptable toxicity. The different dosing schedules will evaluate the effect of different frequencies and dose intensities of the two antibodies on efficacy and safety. In the former schedule, the dose of nivolumab is lower and administration is every 3 weeks during induction, which allows ipilimumab to be administered more frequently, but over a shorter period of time. In the latter schedule, nivolumab becomes the backbone of the combination, as it will be administered more frequently and at a higher dose, while the ipilimumab be administered continuously throughout the schedule but a lower dose.

### Shorter Infusion Times for Nivolumab and Ipilimumab

Long infusion times, especially when multiple agents are administered sequentially to an individual, place a burden on patients and treatment centers. Establishing that nivolumab and ipilimumab can be safely administered using shorter infusion times of 30 minutes duration for nivolumab and ipilimumab in subjects will diminish the burden provided no change in safety profile.

Previous clinical studies of nivolumab monotherapy and ipilimumab monotherapy and the combination of nivolumab and ipilimumab have used a 60 minute infusion duration for nivolumab and 90 minute infusion duration for ipilimumab (1-3 mg/kg dosing for both). However, both nivolumab and ipilimumab have been administered at up to 10 mg/kg with the same infusion duration: nivolumab has been administered safely over 60 minutes at doses ranging up to 10 mg/kg safely over long treatment duration. In Study CA209010, (a Phase 2, randomized, double blinded, dose-ranging study of nivolumab in subjects with advanced/metastatic clear cell RCC) a dose association was observed for infusion site reactions and hypersensitivity reactions (1.7% at 0.3 mg/kg, 3.7% at 2 mg/kg and 18.5% at 10 mg/kg). All the events were grade 1-2 and were manageable.

An infusion duration of 30 minutes for 3 mg/kg nivolumab (30% of the dose provided at 10 mg/kg) is not expected to present any safety concerns compared to the prior experience at 10 mg/kg nivolumab dose infused over a 60 minute duration.

Similarly, ipilimumab at 10 mg/kg has been safely administered over a 90 minute duration. In the CA184022 study, where ipilimumab was administered up to a dose of 10 mg/kg, on-study drug related hypersensitivity events (Grade 1-2) were reported in 1 (1.4%) subject in the 0.3 mg/kg and in 2 (2.8%) subjects in the 10 mg/kg group. There were no drug-related hypersensitivity events reported in the 3 mg/kg group. Across the 3 treatment groups, no Grade 3-4 drug-related hypersensitivity events were reported and there were no reports of infusion reactions. Ipilimumab 10 mg/kg monotherapy has also been safely administered as 90 minute infusion in large phase 3 studies in prostate cancer (CA184043) and as adjuvant therapy for stage 3 melanoma (CA184029), with infusion reactions occurring in subjects. Administering 1 mg/kg of ipilimumab represents one-tenth of the 10 mg/kg dose.

Overall, infusion reactions including high-grade hypersensitivity reactions have been uncommon across nivolumab or ipilimumab clinical studies or the combination of nivolumab and ipilimumab. Furthermore, a 30 minute break after the first infusion for combination cohort will ensure the appropriate safety monitoring before the start of the second infusion. Overall, a change in safety profile is not anticipated with 30 minute infusion of nivolumab, ipilimumab, or combination.

In some embodiments, the anti-cancer agent which is an antibody or an antigen-binding portion thereof that binds specifically to a Programmed Death-1 (PD-1) receptor and inhibits PD-1 activity, e.g., nivolumab, is administered by infusion for less than 60 minutes (e.g., about 30 minutes). In some embodiments, another anti-cancer agent, e.g., ipilimumab, is administered by infusion for less than 90 minutes (e.g., about 60 or about 30 minutes).

### Platinum Doublet Chemotherapy in Comparator Arm (Arm D)

First-line treatment of advanced NSCLC is histology specific. For example, pemetrexed is approved in first-line in combination with cisplatin for locally advanced or metastatic non-squamous NSCLC. This approval was based on a Phase III, randomized study that showed improved survival and decreased toxicity for pemetrexed combined with cisplatin in patients with non-squamous histology, in comparison to gemcitabine combined with cisplatin. (Scagliotti G V et al. J Clin Oncol 2008; 26: 3543-51.)

Pemetrexed has also been approved as continuation maintenance therapy in patients with non-squamous histology, who have not had progressive disease after four cycles of a first-line pemetrexed/platinum regimen. In contrast, gemcitabine in combination with cisplatin has been demonstrated to yield improved overall survival compared to pemetrexed/cisplatin in subjects with squamous NSCLC.

Although some but not all meta-analyses and randomized studies have demonstrated that cisplatin-based regimens may produce improved survival compared to carboplatin-based regimens, many subjects are not ideal candidates for cisplatin due to its higher toxicity. (Azzoli et al. J Clin Oncol 2009; 27: 6251-66.)

In order to accommodate subjects with both squamous and non-squamous histology in this study, subjects with squamous histology randomized to the comparator arm (Arm D) may receive either of the following platinum regimens: gemcitabine/cisplatin (up to 6 cycles) or gemcitabine/carboplatin (up to 6 cycles).

Subjects with non-squamous histology who are randomized to the comparator arm (Arm D) may receive either of the following pemetrexed/platinum regimens, and they have the option for continuation of pemetrexed as maintenance therapy: pemetrexed/cisplatin (up to 6 cycles), pemetrexed/carboplatin (up to 6 cycles), pemetrexed/cisplatin (4 cycles), followed by pemetrexed maintenance, or pemetrexed/carboplatin (4 cycles), followed by pemetrexed maintenance.

### Evaluation of PD-L1 Expression as a Predictive Biomarker

PD-L1 is expressed by many tumor types and its expression has been noted to correlate with decreased immune system function and worse clinical prognosis. It is hypothesized that PD-L1 expression within the tumor microenvironment, either on tumor cells, macrophages or lymphocytes is a means of evading immune system detection and destruction. Still others postulate that PD-L1 expression on tumor cells is a surrogate for interferon-gamma release from neighboring activated T cells and thus portends a good prognosis for immunotherapy agents, and in particular, agents targeting the PD-1/PD-L1 axis.

A prospective assessment of PD-L1 status within tumor biopsies will be conducted. There is evidence to suggest that PD-L1 expression may be both prognostic and predictive. One of the stratification factors will therefore be PD-L1 expression in order to attempt to decrease potential prognostic biases and also increase the ability to evaluate the predictive value of PD-L1 for response to nivolumab.

The PD-L1 status of a tumor in a subject can be measured prior to administering any composition or utilizing any method disclosed herein. PD-L1 expression can be determined by any methods known in the art.

In order to assess the PD-L1 expression, in one embodiment, a test tissue sample can be obtained from the patient who is in need of the therapy. In another embodiment, the assessment of PD-L1 expression can be achieved without obtaining a test tissue sample. In some embodiments, selecting a suitable patient includes (i) optionally providing a test tissue sample obtained from a patient with cancer of the tissue, the test tissue sample comprising tumor cells and/or tumor-infiltrating inflammatory cells; and (ii) assessing the proportion of cells in the test tissue sample that express PD-L1 on the surface of the cells based on an assessment that the proportion of cells in the test tissue sample that express PD-L1 on the cell surface is higher than a predetermined threshold level.

In any of the methods comprising the measurement of PD-L1 expression in a test tissue sample, however, it should be understood that the step comprising the provision of a test tissue sample obtained from a patient is an optional step. It should also be understood that in certain embodiments the “measuring” or “assessing” step to identify, or determine the number or proportion of, cells in the test tissue sample that express PD-L1 on the cell surface is performed by a transformative method of assaying for PD-L1 expression, for example by performing a reverse transcriptase-polymerase chain reaction (RT-PCR) assay or an IHC assay. In certain other embodiments, no transformative step is involved and PD-L1 expression is assessed by, for example, reviewing a report of test results from a laboratory. In certain embodiments, the steps of the methods up to, and including, assessing PD-L1 expression provides an intermediate result that may be provided to a physician or other healthcare provider for use in selecting a suitable candidate for the anti-PD-1 antibody or anti-PD-L1 antibody therapy. In certain embodiments, the steps that provide the intermediate result is performed by a medical practitioner or someone acting under the direction of a medical practitioner. In other embodiments, these steps are performed by an independent laboratory or by an independent person such as a laboratory technician.

In certain embodiments of any of the present methods, the proportion of cells that express PD-L1 is assessed by performing an assay to determine the presence of PD-L1 RNA. In further embodiments, the presence of PD-L1 RNA is determined by RT-PCR, in situ hybridization or RNase protection. In other embodiments, the proportion of cells that express PD-L1 is assessed by performing an assay to determine the presence of PD-L1 polypeptide. In further embodiments, the presence of PD-L1 polypeptide is determined by immunohistochemistry (IHC), enzyme-linked immunosorbent assay (ELISA), in vivo imaging, or flow cytometry. In some embodiments, PD-L1 expression is assayed by IHC. In other embodiments of all of these methods, cell surface expression of PD-L1 is assayed using, e.g., IHC or in vivo imaging.

Imaging techniques have provided important tools in cancer research and treatment. Recent developments in molecular imaging systems, including positron emission tomography (PET), single-photon emission computed tomography (SPECT), fluorescence reflectance imaging (FRI), fluorescence-mediated tomography (FMT), bioluminescence imaging (BLI), laser-scanning confocal microscopy (LSCM) and multiphoton microscopy (MPM), will likely herald even greater use of these techniques in cancer research. Some of these molecular imaging systems allow clinicians to not only see where a tumor is located in the body, but also to visualize the expression and activity of specific molecules, cells, and biological processes that influence tumor behavior and/or responsiveness to therapeutic drugs (Condeelis and Weissleder, “In vivo imaging in cancer,” Cold Spring Harb. Perspect. Biol. 2(12): a003848 (2010)). Antibody specificity, coupled with the sensitivity and resolution of PET, makes immunoPET imaging particularly attractive for monitoring and assaying expression of antigens in tissue samples (McCabe and Wu, “Positive progress in immunoPET—not just a coincidence,” Cancer Biother. Radiopharm. 25(3):253-61 (2010); Olafsen et al., “ImmunoPET imaging of B-cell lymphoma using 124I-anti-CD20 scFv dimers (diabodies),” Protein Eng. Des. Sel. 23(4):243-9 (2010)). In certain embodiments of any of the present methods, PD-L1 expression is assayed by immunoPET imaging. In certain embodiments of any of the present methods, the proportion of cells in a test tissue sample that express PD-L1 is assessed by performing an assay to determine the presence of PD-L1 polypeptide on the surface of cells in the test tissue sample. In certain embodiments, the test tissue sample is a FFPE tissue sample. In other embodiments, the presence of PD-L1 polypeptide is determined by IHC assay. In further embodiments, the IHC assay is performed using an automated process. In some embodiments, the IHC assay is performed using an anti-PD-L1 monoclonal antibody to bind to the PD-L1 polypeptide.

In one embodiment of the present methods, an automated IHC method is used to assay the expression of PD-L1 on the surface of cells in FFPE tissue specimens. This disclosure provides methods for detecting the presence of human PD-L1 antigen in a test tissue sample, or quantifying the level of human PD-L1 antigen or the proportion of cells in the sample that express the antigen, which methods comprise contacting the test sample, and a negative control sample, with a monoclonal antibody that specifically binds to human PD-L1, under conditions that allow for formation of a complex between the antibody or portion thereof and human PD-L1. In certain embodiments, the test and control tissue samples are FFPE samples. The formation of a complex is then detected, wherein a difference in complex formation between the test sample and the negative control sample is indicative of the presence of human PD-L1 antigen in the sample. Various methods are used to quantify PD-L1 expression.

In a particular embodiment, the automated IHC method comprises: (a) deparaffinizing and rehydrating mounted tissue sections in an autostainer; (b) retrieving antigen using a decloaking chamber and pH 6 buffer, heated to 110° C. for 10 min; (c) setting up reagents on an autostainer; and (d) running the autostainer to include steps of neutralizing endogenous peroxidase in the tissue specimen; blocking non-specific protein-binding sites on the slides; incubating the slides with primary antibody; incubating with a post primary blocking agent; incubating with NovoLink Polymer; adding a chromogen substrate and developing; and counterstaining with hematoxylin.

For assessing PD-L1 expression in tumor tissue samples, a pathologist examines the number of membrane PD-L1⁺ tumor cells in each field under a microscope and mentally estimates the percentage of cells that are positive, then averages them to come to the final percentage. The different staining intensities are defined as 0/negative, 1+/weak, 2+/moderate, and 3+/strong. Typically, percentage values are first assigned to the 0 and 3+ buckets, and then the intermediate 1+ and 2+ intensities are considered. For highly heterogeneous tissues, the specimen is divided into zones, and each zone is scored separately and then combined into a single set of percentage values. The percentages of negative and positive cells for the different staining intensities are determined from each area and a median value is given to each zone. A final percentage value is given to the tissue for each staining intensity category: negative, 1+, 2+, and 3+. The sum of all staining intensities needs to be 100%. In one embodiment, the threshold number of cells that needs to be PD-L1 positive is at least about 100, at least about 125, at least about 150, at least about 175, or at least about 200 cells. In certain embodiments, the threshold number or cells that needs to be PD-L1 positive is at least about 100 cells.

Staining is also assessed in tumor-infiltrating inflammatory cells such as macrophages and lymphocytes. In most cases macrophages serve as an internal positive control since staining is observed in a large proportion of macrophages. While not required to stain with 3+ intensity, an absence of staining of macrophages should be taken into account to rule out any technical failure. Macrophages and lymphocytes are assessed for plasma membrane staining and only recorded for all samples as being positive or negative for each cell category. Staining is also characterized according to an outside/inside tumor immune cell designation. “Inside” means the immune cell is within the tumor tissue and/or on the boundaries of the tumor region without being physically intercalated among the tumor cells. “Outside” means that there is no physical association with the tumor, the immune cells being found in the periphery associated with connective or any associated adjacent tissue.

In certain embodiments of these scoring methods, the samples are scored by two pathologists operating independently, and the scores are subsequently consolidated. In certain other embodiments, the identification of positive and negative cells is scored using appropriate software.

A histoscore is used as a more quantitative measure of the IHC data. The histoscore is calculated as follows:

Histoscore=[(% tumor×1(low intensity))+(% tumor×2(medium intensity))+(% tumor×3(high intensity)]

To determine the histoscore, the pathologist estimates the percentage of stained cells in each intensity category within a specimen. Because expression of most biomarkers is heterogeneous the histoscore is a truer representation of the overall expression. The final histoscore range is 0 (no expression) to 300 (maximum expression).

An alternative means of quantifying PD-L1 expression in a test tissue sample IHC is to determine the adjusted inflammation score (AIS) score defined as the density of inflammation multiplied by the percent PD-L1 expression by tumor-infiltrating inflammatory cells (Taube et al., “Colocalization of inflammatory response with B7-hl expression in human melanocytic lesions supports an adaptive resistance mechanism of immune escape,” Sci. Transl. Med. 4(127):127ra37 (2012)).

In one embodiment, the PD-L1 expression level of a tumor is at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 40%, at least about 50%, at least about 60%, at least about 70%, at least about 75%, at least about 80%, at least about 85%, at least about 90%, at least about 95%, or about 100%. In another embodiment, the PD-L1 status of a tumor is at least about 1%. In other embodiments, the PD-L1 status of the subject is at least about 5%. In a certain embodiment, the PD-L1 status of a tumor is at least about 10%. In a one embodiment, the PD-L1 status of the tumor is at least about 25%. In a particular embodiment, the PD-L1 status of the tumor is at least about 50%.

In some embodiments, the present disclosure includes methods of treating a MSI-high (MSI-H) tumor, a MSI stable tumor, or a MSI low (MSI-L) tumor, e.g., a colorectal tumor, comprising administering the combination therapy of an anti-PD-1 antibody and an anti-CD27 antibody to a subject that has a tumor expressing PD-L1 or a PD-L1 positive tumor. In certain embodiments, the present disclosure is directed to a method of treating a tumor, e.g., a colorectal tumor, comprising (i) identifying a subject who has a MSI-high (MSI-H) tumor, a MSI stable tumor, or a MSI low (LSI-L) tumor; (ii) assessing whether the tumor expresses PD-L1; and (iii) administering an effective amount of an anti-PD-1 antibody and an effective amount of an anti-CD27 antibody to the subject. In certain embodiments, the subject has a tumor that has ≥1% PD-L1 expression, ≥5% PD-L1 expression, ≥10% PD-L1 expression, ≥25% PD-L1 expression, or ≥50% PD-L1 expression.

In another embodiment, the present disclosure provides a method of treating a tumor, e.g., a colorectal tumor, comprising (i) identifying a subject who has a MSI-high (MSI-H) tumor, a MSI stable tumor, or a MSI low (LSI-L) tumor; (ii) assessing whether the tumor is PD-L1 positive; and (iii) administering an effective amount of an anti-PD-1 antibody and an effective amount of an anti-CD27 antibody to the subject.

“PD-L1 positive” as used herein can be interchangeably used with “PD-L1 expression of at least about 1%”. In one embodiment, the PD-L1 positive tumors can thus have at least about 1%, at least about 2%, at least about 5%, at least about 10%, at least about 20%, at least about 25%, at least about 30%, at least about 40%, at least about 50%, at least about 60%, at least about 70%, at least about 75%, at least about 80%, at least about 85%, at least about 90%, at least about 95%, or about 100% of the tumor cells expressing PD-L1 as measured by an automated IHC. In certain embodiments, “PD-L1 positive” means that there are at least 100 cells that express PD-L1 on the surface of the cells.

### Including PD-L1 Positive and Negative Tumors

Analyses based on archival specimens for subjects in CA209012 have shown that subjects with both PD-L1+(IHC greater than or equal to 5%) and PD-L1 tumors may benefit from treatment with nivolumab. The overall response rate (ORR) for subjects with PD-L1+ tumors who received nivolumab monotherapy (n=26) was 31%, but the response rate for those with PD-L1- tumors (n=20) was 15%. However, the disease control rate (DCR, CR+PR+SD) was 54% for those with PD-L1+ tumors and 50% for those with PD-L1− tumors. Pooled data for all subjects who received the combination of nivolumab+ipilimumab showed an ORR of 23% for those with PD-L1+ tumors (n=30) and 11% for those with PD-L1− tumors (n=37). Again there was less of a difference in the DCR between the 2 groups: 53% for those with PD-L1+ tumors and 51% for those with PD-L1− tumors. For subjects receiving nivolumab monotherapy, the PFS rate at 24 weeks was 40% and 45% for patients with PD-L1+ and PD-L1− tumors respectively; the median OS was 19.1 months in subjects with PD-L1+ tumors and not reached in subjects with PD-L1− tumors. For subjects receiving nivolumab plus ipilimumab, the median PFS was 21.7 weeks and 12.4 weeks in PD-L1+ and PD-L1− tumors respectively; the median OS was 21.5 months in subjects with PD-L1+ tumors and 19.4 months in subjects with PD-L1− tumors.

Thus, subjects with PD-L1+ and PD-L1− tumors will be eligible to participate in CA209227.

Exclusion of Subjects with EGFR Mutations or ALK Translocations

As first-line standard of care for subjects with EGFR mutations and ALK translocations is targeted therapy rather than chemotherapy, subjects known to have these abnormalities will be excluded from this study.

In addition, patients with EGFR mutations have a better prognosis, even in the absence of EGFR inhibitor therapy (Eberhard, D A, et al. J Clin Onc. 2005; 2: 5900-5909) and may have an improved response to chemotherapy compared to patients without EGFR mutations (Mok T S, et al. N Engl J Med 2009; 361-947-57). Patients with ALK translocations who are treated with chemotherapy appear to have similar PFS compared to patients without ALK translocations who are treated with chemotherapy. (Shaw A T, et al. Annals of Oncology 2013; 24: 59-66.) Excluding subjects with these abnormalities will help to reduce the potentially confounding effects of these abnormalities on the study endpoints.

### Overall Survival in Comparator Arm

Prior phase 3 randomized studies of platinum double chemotherapy have documented median OS of 9-11 month. More recent randomized studies with maintenance therapy with bevacizumab or pemetrexed in non-squamous NSCLC patients have further improved median OS to ˜13.9 month. As this study will enroll NSCLC patients regardless of histology, we anticipate that about 80% subjects will have non-squamous histology, and that about half of these subjects are expected to receive continuation maintenance therapy.

Also, nivolumab is approved in the U.S. as second-line therapy for patients with metastatic squamous NSCLC with progression on or after platinum-based chemotherapy. It may be approved in other countries during the conduct of this study. The number of subjects who receive treatment with nivolumab after progression depends on whether and when approval will be granted in each country. We estimate that about 30% the subjects randomized to the platinum doublet chemotherapy arm are expected to receive immunotherapy (e.g., nivolumab) as second-line therapy after progression, which will further improve median OS in this arm. For these reasons, the median OS is estimated to be 13 months for the comparator arm (Arm D).

### Open Label Design

This study uses an open-label design. Due to the obvious difference in chemotherapy and immunotherapy related toxicities, histology-dependent chemotherapy options, the different schedules and durations of therapy in the treatment arms, different dose modification rules for safety management, including different dose delay rules per arm, and different pre-medication requirements according to chemotherapy, an open-label design is appropriate. An open-label design will also help ensure that immune-related toxicities in subjects receiving immunotherapy are promptly identified and managed.

Because this study is open label, a blinded independent central review (BICR) will be used to review tumor images in all randomized subjects to determine all response-related endpoints.

### Continued Treatment in Select Cases of Progressive Disease

Accumulating clinical evidence indicates some subjects treated with immune system stimulating agents may develop progression of disease (by conventional response criteria) before demonstrating clinical objective responses and/or stable disease. This phenomenon was observed in approximately 10% of subjects in the Phase 1 study of nivolumab and also with ipilimumab monotherapy. (Wolchok J D, et al. Clin Cancer Res. 2009; 15:7412-20.) Without being bound by theory, two hypotheses are put forth to explain this phenomenon. First, enhanced inflammation within tumors could lead to an increase in tumor size which would appear as enlarged index lesions and as newly visible small non-index lesions. Over time, both the malignant and inflammatory portions of the mass may then decrease leading to overt signs of clinical improvement. Alternatively, in some individuals, the kinetics of tumor growth may initially outpace anti-tumor immune activity. With sufficient time, the anti-tumor activity will dominate and become clinically apparent. Therefore for Arms A, B and C, subjects will be allowed to continue study therapy after initial investigator-assessed RECIST 1.1 defined progression if they are assessed to be deriving clinical benefit and tolerating study drug. Such subjects must discontinue study therapy upon evidence of further progression.

### Recist 1.1 Guidelines

Evaluation of Lesions

At baseline, tumor lesions/lymph nodes will be categorized measurable or non-measurable as follows:

Measurable Tumor lesions: Must be accurately measured in at least one dimension (longest diameter in the plane of measurement is to be recorded) with a minimum size of:

1. 10 mm by CT scan (CT scan slice thickness no greater than 5 mm)

2. 10 mm caliper measurement by clinical exam (lesions which cannot be accurately measured with calipers should be recorded as non-measurable)

3. 20 mm by chest x-ray

Measurable Malignant lymph nodes: To be considered pathologically enlarged and measurable, a lymph node must be greater than or equal to 15 mm in short axis when assessed by CT scan (CT scan slice thickness recommended to be no greater than 5 mm).

Measurable Lymph nodes merit special mention since they are normal anatomical structures which may be visible by imaging even if not involved by tumor. Pathological nodes which are defined as measurable and may be identified as target lesions must meet the criterion of a short axis of greater than or equal to 15 mm by CT scan. Only the short axis of these nodes will contribute to the baseline sum. The short axis of the node is the diameter normally used by radiologists to judge if a node is involved by solid tumor. Nodal size is normally reported as two dimensions in the plane in which the image is obtained (for CT scan this is almost always the axial plane; for MM the plane of acquisition may be axial, sagittal or coronal). The smaller of these measures is the short axis. For example, an abdominal node which is reported as being 20 mm×30 mm has a short axis of 20 mm and qualifies as a malignant, measurable node. In this example, 20 mm should be recorded as the node measurement. All other pathological nodes (those with short axis greater than or equal to 10 mm but <15 mm) should be considered non-target lesions. Nodes that have a short axis <10 mm are considered non-pathological and should not be recorded or followed.

All other lesions are considered non-measurable, including small lesions (longest diameter <10 mm or pathological lymph nodes with greater than or equal to 10 to <15 mm short axis) as well as truly non-measurable lesions. Lesions considered truly non-measurable include: leptomeningeal disease, ascites, pleural or pericardial effusion, inflammatory breast disease, lymphangitic involvement of skin or lung, abdominal masses/abdominal organomegaly identified by physical exam that is not measurable by reproducible imaging techniques.

When more than one measurable lesion is present at baseline all lesions up to a maximum of five lesions total (and a maximum of two lesions per organ) representative of all involved organs should be identified as target lesions and will be recorded and measured at baseline (this means in instances where patients have only one or two organ sites involved a maximum of two and four lesions respectively will be recorded).

Target lesions should be selected on the basis of their size (lesions with the longest diameter), be representative of all involved organs, but in addition should be those that lend themselves to reproducible repeated measurements. It may be the case that, on occasion, the largest lesion does not lend itself to reproducible measurement in which circumstance the next largest lesion which can be measured reproducibly should be selected.

A sum of the diameters (longest for non-nodal lesions, short axis for nodal lesions) for all target lesions will be calculated and reported as the baseline sum diameters. If lymph nodes are to be included in the sum, then as noted above, only the short axis is added into the sum. The baseline sum diameters will be used as reference to further characterize any objective tumor regression in the measurable dimension of the disease.

All other lesions (or sites of disease) including pathological lymph nodes should be identified as non-target lesions and should also be recorded at baseline. Measurements are not required and these lesions should be followed as ‘present’, ‘absent’, or in rare cases ‘unequivocal progression’ (more details to follow). In addition, it is possible to record multiple non-target lesions involving the same organ as a single item on the case record form (e.g., ‘multiple enlarged pelvic lymph nodes’ or ‘multiple liver metastases’).

Evaluation of Target Lesions

Complete Response (CR): Disappearance of all target lesions. Any pathological lymph nodes (whether target or non-target) must have reduction in short axis to <10 mm.

Partial Response (PR): At least a 30% decrease in the sum of diameters of target lesions, taking as reference the baseline sum diameters.

Progressive Disease (PD): At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression).

Stable Disease (SD): Neither sufficient shrinkage to qualify for PR nor sufficient increase to qualify for PD, taking as reference the smallest sum diameters while on study.

Lymph Nodes

Lymph nodes identified as target lesions should always have the actual short axis measurement recorded (measured in the same anatomical plane as the baseline examination), even if the nodes regress to below 10 mm on study. This means that when lymph nodes are included as target lesions, the ‘sum’ of lesions may not be zero even if complete response criteria are met, since a normal lymph node is defined as having a short axis of <10 mm. Case report forms or other data collection methods may therefore be designed to have target nodal lesions recorded in a separate section where, in order to qualify for CR, each node must achieve a short axis <10 mm. For PR, SD and PD, the actual short axis measurement of the nodes is to be included in the sum of target lesions.

Target Lesions that Become ‘Too Small to Measure’

While on study, all lesions (nodal and non-nodal) recorded at baseline should have their actual measurements recorded at each subsequent evaluation, even when very small (e.g., 2 mm). However, sometimes lesions or lymph nodes which are recorded as target lesions at baseline become so faint on CT scan that the radiologist may not feel comfortable assigning an exact measure and may report them as being ‘too small to measure’. When this occurs it is important that a value be recorded on the case report form. If it is the opinion of the radiologist that the lesion has likely disappeared, the measurement should be recorded as 0 mm. If the lesion is believed to be present and is faintly seen but too small to measure, a default value of 5 mm should be assigned (Note: It is less likely that this rule will be used for lymph nodes since they usually have a definable size when normal and are frequently surrounded by fat such as in the retroperitoneum; however, if a lymph node is believed to be present and is faintly seen but too small to measure, a default value of 5 mm should be assigned in this circumstance as well). This default value is derived from the 5 mm CT slice thickness (but should not be changed with varying CT slice thickness). The measurement of these lesions is potentially non-reproducible, therefore providing this default value will prevent false responses or progressions based upon measurement error. To reiterate, however, if the radiologist is able to provide an actual measure, that should be recorded, even if it is below 5 mm.

Lesions that Split or Coalesce on Treatment

When non-nodal lesions ‘fragment’, the longest diameters of the fragmented portions should be added together to calculate the target lesion sum. Similarly, as lesions coalesce, a plane between them may be maintained that would aid in obtaining maximal diameter measurements of each individual lesion. If the lesions have truly coalesced such that they are no longer separable, the vector of the longest diameter in this instance should be the maximal longest diameter for the ‘coalesced lesion’.

Evaluation of Non-Target Lesions

This section provides the definitions of the criteria used to determine the tumor response for the group of non-target lesions. While some non-target lesions may actually be measurable, they need not be measured and instead should be assessed only qualitatively at the time points specified in the protocol.

Complete Response (CR): Disappearance of all non-target lesions and normalization of tumor marker level. All lymph nodes must be non-pathological in size (<10 mm short axis).

Non-CR/Non-PD: Persistence of one or more non-target lesion(s) and/or maintenance of tumor marker level above the normal limits.

Progressive Disease (PD): Unequivocal progression (see comments below) of existing non-target lesions. (Note: the appearance of one or more new lesions is also considered progression).

Assessment of Progression of Non-Target Disease

The concept of progression of non-target disease requires additional explanation as follows:

When the patient also has measurable disease, to achieve ‘unequivocal progression’ on the basis of the non-target disease, there must be an overall level of substantial worsening in non-target disease such that, even in presence of SD or PR in target disease, the overall tumor burden has increased sufficiently to merit discontinuation of therapy (see examples in Appendix 2 and further details below). A modest ‘increase’ in the size of one or more non-target lesions is usually not sufficient to qualify for unequivocal progression status. The designation of overall progression solely on the basis of change in non-target disease in the face of SD or PR of target disease will therefore be extremely rare.

When the patient has only non-measurable disease, this circumstance arises in some trials when it is not a criterion of study entry to have measurable disease. The same general concepts apply here as noted above, however, in this instance there is no measurable disease assessment to factor into the interpretation of an increase in non-measurable disease burden. Because worsening in non-target disease cannot be easily quantified (by definition: if all lesions are truly non-measurable) a useful test that can be applied when assessing patients for unequivocal progression is to consider if the increase in overall disease burden based on the change in non-measurable disease is comparable in magnitude to the increase that would be required to declare PD for measurable disease: i.e., an increase in tumor burden representing an additional 73% increase in ‘volume’ (which is equivalent to a 20% increase diameter in a measurable lesion). Examples include an increase in a pleural effusion from ‘trace’ to ‘large’, an increase in lymphangitic disease from localized to widespread, or may be described in protocols as ‘sufficient to require a change in therapy’. If ‘unequivocal progression’ is seen, the patient should be considered to have had overall PD at that point. While it would be ideal to have objective criteria to apply to non-measurable disease, the very nature of that disease makes it impossible to do so; therefore the increase must be substantial.

New Lesions

The appearance of new malignant lesions denotes disease progression; therefore, some comments on detection of new lesions are important. There are no specific criteria for the identification of new radiographic lesions; however, the finding of a new lesion should be unequivocal: i.e., not attributable to differences in scanning technique, change in imaging modality or findings thought to represent something other than tumor (for example, some ‘new’ bone lesions may be simply healing or flare of pre-existing lesions). This is particularly important when the patient's baseline lesions show partial or complete response. For example, necrosis of a liver lesion may be reported on a CT scan report as a ‘new’ cystic lesion, which it is not.

A lesion identified on a follow-up study in an anatomical location that was not scanned at baseline is considered a new lesion and will indicate disease progression. An example of this is the patient who has visceral disease at baseline and while on study has a CT or MRI brain ordered which reveals metastases. The patient's brain metastases are considered to be evidence of PD even if he/she did not have brain imaging at baseline.

If a new lesion is equivocal, for example because of its small size, continued therapy and follow-up evaluation will clarify if it represents truly new disease. If repeat scans confirm there is definitely a new lesion, then progression should be declared using the date of the initial scan. While FDG-PET response assessments need additional study, it is sometimes reasonable to incorporate the use of FDG-PET scanning to complement CT scanning in assessment of progression (particularly possible ‘new’ disease). New lesions on the basis of FDG-PET imaging can be identified according to the following algorithm:

1. Negative FDG-PET at baseline, with a positive FDG-PET at follow-up is a sign of PD based on a new lesion.

2. No FDG-PET at baseline and a positive FDG-PET at follow-up: If the positive FDG-PET at follow-up corresponds to a new site of disease confirmed by CT, this is PD. If the positive FDG-PET at follow-up is not confirmed as a new site of disease on CT, additional follow-up CT scans are needed to determine if there is truly progression occurring at that site (if so, the date of PD will be the date of the initial abnormal FDG-PET scan). If the positive FDG-PET at follow-up corresponds to a pre-existing site of disease on CT that is not progressing on the basis of the anatomic images, this is not PD.

Response Assessment

The best overall response is the best response recorded from the start of the study treatment until the end of treatment taking into account any requirement for confirmation. The patient's best overall response assignment will depend on the findings of both target and non-target disease and will also take into consideration the appearance of new lesions. Furthermore, depending on the nature of the study and the protocol requirements, it may also require confirmatory measurement.

Time Point Response

It is assumed that at each protocol specified time point, a response assessment occurs. Table 4 provides a summary of the overall response status calculation at each time point for patients who have measurable disease at baseline. When patients have non-measurable (therefore non-target) disease only, Table 5 is to be used.

TABLE 4
Time Point Response: Patients With Target
(+/−Non-Target) Disease

| Target Lesions | Non-Target Lesions | New Lesions | Overall Response |
|---|---|---|---|
| CR | CR | No | CR |
| CR | Non-CR/non-PD | No | PR |
| CR | Not evaluated | No | PR |
| PR | Non-PD or not all | No | PR |
| | evaluated | | |
| SD | Non-PD or not all | No | SD |
| | evaluated | | |
| Not all evaluated | Non-PD | No | NE |
| PD | Any | Yes or No | PD |
| Any | PD | Yes or No | PD |
| Any | Any | Yes | PD |

CR = complete response,
PR = partial response,
SD = stable disease,
PD = progressive disease, and
NE = inevaluable

TABLE 5
Time Point Response: Patients with Non-target Disease Only

| Non-Target Lesions | New Lesions | Overall Response |
|---|---|---|
| CR | No | CR |
| Non-CR/non-PD | No | Non-CR/non-PD^{a} |
| Not all evaluated | No | NE |
| Unequivocal PD | Yes or No | PD |
| Any | Yes | PD |

CR = complete response,
PD = progressive disease and
NE = inevaluable
^{a}Non-CR/non-PD is preferred over SD for non-target disease since SD is increasingly used as endpoint for assessment of efficacy in some trials so to assign this category when no lesions can be measured is not advised.

Best response determination of complete or partial response requires confirmation: Complete or partial responses may be claimed only if the criteria for each are met at a subsequent time point of greater than or equal to 4 weeks later. In this circumstance, the best overall response can be interpreted as in Table 6.

TABLE 6
Best Overall Response (Confirmation of CR&PR Required)

| Overall | Overall Response | |
|---|---|---|
| Response | Subsequent Time | BEST Overall Response |
| CR | CR | CR |
| CR | PR | SD, PD OR PR^{a} |
| CR | SD | SD provided minimum criteria for |
| | | SD^{b} duration met, otherwise, PD |
| CR | PD | SD provided minimum criteria for |
| | | SD^{b} duration met, otherwise, PD |
| CR | NE | SD provided minimum criteria for |
| | | SD^{b} duration met, otherwise, NE |
| PR | CR | PR |
| PR | PR | PR |
| PR | SD | SD |
| PR | PD | SD provided minimum criteria for |
| | | SD^{b} duration met, otherwise, PD |
| PR | NE | SD provided minimum criteria for |
| | | SD^{b} duration met, otherwise, NE |
| NE | NE | NE |

CR = complete response,
PR = partial response,
SD = stable disease,
PD = progressive disease, and
NE = inevaluable
^{a}If a CR is truly met at first time point, then any disease seen at a subsequent time point, even disease meeting PR criteria relative to baseline, makes the disease PD at that point (since disease must have reappeared after CR). Best response would depend on whether minimum duration for SD was met. However, sometimes ‘CR’ may be claimed when subsequent scans suggest small lesions were likely still present and in fact the patient had PR, not CR at the first time point. Under these circumstances, the original CR should be changed to PR and the best response is PR.
^{b}Minimum criteria for SD duration is 6 weeks.

When nodal disease is included in the sum of target lesions and the nodes decrease to ‘normal’ size (<10 mm), they may still have a measurement reported on scans. This measurement should be recorded even though the nodes are normal in order not to overstate progression should it be based on increase in size of the nodes. As noted earlier, this means that patients with CR may not have a total sum of ‘zero’ on the case report form (CRF).

Confirmation Scans

Verification of Response: To be assigned a status of CR or PR, changes in tumor measurements must be confirmed by consecutive repeat assessments that should be performed no less than 28 days after the criteria for response are first met. For this study, the next scheduled tumor assessment can meet this requirement.

Verification of Progression: Progression of disease should be verified in cases where progression is equivocal. If repeat scans confirm PD, then progression should be declared using the date of the initial scan. If repeat scans do not confirm PD, then the subject is considered to not have progressive disease.

### Study Objectives

Some embodiments are directed to a comparison of the overall survival (OS) of nivolumab monotherapy and nivolumab in combination with ipilimumab to platinum-doublet chemotherapy in subjects with previously untreated stage IV or recurrent NSCLC.

Some embodiments are directed to a comparison of the progression-free survival (PFS), based on BICR assessment of nivolumab monotherapy and nivolumab in combination with ipilimumab, to platinum-doublet chemotherapy in subjects with previously untreated stage IV or recurrent NSCLC.

Some embodiments are directed to a comparison of the objective response rate (ORR), based on BICR assessment of nivolumab and nivolumab in combination with ipilimumab, to platinum-doublet chemotherapy in patients with previously untreated stage IV or recurrent NSCLC.

Some embodiments are directed to a pairwise comparison of OS among experimental arms.

Some embodiments are directed to differences in PFS and ORR between nivolumab combined with ipilimumab and nivolumab monotherapy in subjects with stage IV or recurrent NSCLC.

Some embodiments are directed PD-L1 expression as a predictive biomarker for OS or PFS.

Some embodiments are directed to treating patients exhibiting disease-related symptom improvement by 12 weeks as measured by the Lung Cancer Symptom Score (LCSS), e.g., subjects receiving nivolumab monotherapy, nivolumab in combination with ipilimumab or subjects receiving platinum doublet chemotherapy.

Some embodiments are directed to safety and tolerability of nivolumab and nivolumab in combination with ipilimumab compared to platinum-doublet chemotherapy.

Some embodiments are directed to pharmacokinetics of nivolumab in combination with ipilimumab and exposure-safety and exposure-efficacy relationships.

Some embodiments are directed to immunogenicity of nivolumab in combination with ipilimumab.

Some embodiments are directed to immune correlates of nivolumab, nivolumab in combination with ipilimumab and platinum-doublet chemotherapy.

Some embodiments are directed to predictive tumor and peripheral biomarkers of clinical response to nivolumab and nivolumab in combination with ipilimumab.

Some embodiments are directed to a comparison of overall health status using the EQ-5D index and visual analogue scale in subjects treated with nivolumab in combination with ipilimumab and in those treated with platinum doublet chemotherapy.

### Mechanisms of Action of Nivolumab

Cancer immunotherapy rests on the premise that tumors can be recognized as foreign rather than as self and can be effectively attacked by an activated immune system. An effective immune response in this setting is thought to rely on immune surveillance of tumor antigens expressed on cancer cells that ultimately results in an adaptive immune response and cancer cell death. Meanwhile, tumor progression may depend upon acquisition of traits that allow cancer cells to evade immunosurveillance and escape effective innate and adaptive immune responses. (Pardoll D M, Nature 2012; 12:252-64; Zitvogel L, Tesniere A, Kroemer G. Nat Rev Immunol. 2006; 6:715-27; and Dunn G P, et al. Nat Immunol. 2002; 3:991-8.)

Support for the role of immunosurveillance in NSCLC is suggested in retrospective analyses demonstrating a correlation between tumor infiltrating lymphocytes in surgically resected specimens and recurrence free survival. (Home Z D et al. J Surg Res. 2011; 171:1-5; Al-Shibli K I et al. Clin Cancer Res. 2008; 14:5220-7; and Ruffini E et al. Ann Thorac Surg. 2009; 87:365-72.) Current immunotherapy efforts attempt to break the apparent tolerance of the immune system to tumor cells and antigens by either introducing cancer antigens by therapeutic vaccination or by modulating regulatory checkpoints of the immune system.

T-cell stimulation is a complex process involving the integration of numerous positive as well as negative co-stimulatory signals in addition to antigen recognition by the T-cell receptor (TCR). (Greenwald R J, Freeman G H, Sharpe A H. Annu Rev Immunol. 2004; 23:515-48.)

Collectively, these signals govern the balance between T-cell activation and tolerance to antigens. PD-1 is a member of the CD28 family of T-cell co-stimulatory receptors that also includes CD28, CTLA-4, ICOS, and BTLA.40 PD-1 signaling has been shown to inhibit CD-28-mediated upregulation of IL-2, IL-10, IL-13, interferon-γ (IFN-γ) and Bcl-xL. PD-1 expression also been noted to inhibit T cell activation, and expansion of previously activated cells. Evidence for a negative regulatory role of PD-1 comes from studies of PD-1 deficient mice, which develop a variety of autoimmune phenotypes.41 These results suggest that PD-1 blockade has the potential to activate anti-self T-cell responses, but these responses are variable and dependent upon various host genetic factors. Thus, PD-1 deficiency or inhibition is not accompanied by a universal loss of tolerance to self-antigens.

In vitro, nivolumab binds to PD-1 with high affinity (EC50 0.39-2.62 nM), and inhibits the binding of PD-1 to its ligands PD-L1 and PD-L2 (IC50±1 nM). BMS-936558 binds specifically to PD-1 and not to related members of the CD28 family such as CD28, ICOS, CTLA-4 and BTLA. Blockade of the PD-1 pathway by nivolumab results in a reproducible enhancement of both proliferation and IFN-γ release in the mixed lymphocyte reaction (MLR). Using a CMV re-stimulation assay with human PBMC, the effect of nivolumab on antigen specific recall response indicates that nivolumab augmented IFN-γ secretion from CMV specific memory T cells in a dose-dependent manner versus isotype-matched control. In vivo blockade of PD-1 by a murine analog of nivolumab enhances the anti-tumor immune response and result in tumor rejection in several immunocompetent mouse tumor models (MC38, SA1/N, and PAN02). (Topalian S L et al. N Eng J Med 2012; 366: 2443-54.)

### Cytotoxic T-Lymphocyte Antigen 4 (CTLA-4) and Ipilimumab

CTLA-4, an activation-induced T-cell surface molecule, is a member of the CD28:B7 immunoglobulin superfamily that competes with CD28 for B7. CTLA-4 mediated signals are inhibitory and turn off T cell-dependent immune responses. (Alegre M L et al. J Immunol 1996; 157:4762-70; Postow M A, Harding J, Wolchok J D. Cancer J 2012; 18:153-159.)

Ipilimumab is a fully human monoclonal IgG1κ that binds to the CTLA-4 antigen expressed on a subset of T cells from human and nonhuman primates. A proposed mechanism of action for ipilimumab is interference of the interaction of CTLA-4 with B7 molecules on APCs, with subsequent blockade of the inhibitory modulation of T-cell activation promoted by the CTLA-4/B7 interaction.

### Non-Small Cell Lung Cancer (NSCLC)

Lung cancer is the leading cause of cancer and cancer-related deaths globally, accounting for 1.8 million new cases and 1.6 million deaths worldwide in 2012. (Ferlay J, et al. GLOBOCAN 2012 v1.0, Cancer Incidence and Mortality Worldwide: IARC CancerBase No. 11 [Internet]. Lyon, France: International Agency for Research on Cancer; 2013.) Between 2004 and 2010, according to the SEER database, the overall 5 year survival rate was 21.4%. (Surveillance, Epidemiology, and End Results (SEER) Program Research Data (1973-2011), National Cancer Institute, DCCPS, Surveillance Research Program, Surveillance Systems Branch, released April 2014, based on the November 2013 submission.) The majority of subjects were diagnosed with advanced or metastatic disease. Prognosis for these patients remains dismal, with 5-year survival rates of <5%. Approximately 85% of lung cancer is NSCLC, and of these, approximately 80% are non-squamous, and 20% are squamous histology.

The use of platinum-based chemotherapy doublets, given for up to 6 cycles, is standard-of-care for patients with newly diagnosed advanced or metastatic NSCLC who do not have EGFR mutation or ALK translocation. Current first-line chemotherapy doublets include cisplatin or carboplatin in combination with antimicrotubule agents, gemcitabine, or pemetrexed.

Overall response rates with these platinum doublets are approximately 25%. Progression-free survival (PFS) has remained about 4 to 5.5 months, with overall survival (OS) about 9 to 11 months. (Belani C P et al. J Clin Oncol 2008; 26: 468-73; Scagliotti G V et al. J Clin Oncol 2008; 26: 3543-51.)

Response and outcome after treatment may vary according to histologic subtype. The platinum doublet of pemetrexed/cisplatin improves PFS and OS compared to gemcitabine/cisplatin in subjects with non-squamous histology NSCLC; gemcitabine/cisplatin improves OS compared to pemetrexed/cisplatin in subjects with squamous cell histology. (Scagliotti G V, et al. 2008.) In the PARAMOUNT study, pemetrexed was demonstrated to improve PFS and OS, when continued as maintenance therapy in patients with non-squamous NSCLC which did not progress, after completion of induction treatment with pemetrexed/cisplatin. (Paz-Ares L G et al. J Clin Oncol 2013.) However, there have been no substantial improvements in long term survival, making NSCLC a persistent area of high unmet medical need.

The major adverse events related to platinum doublet chemotherapy regimens are primarily hematologic. For example, with gemcitabine/cisplatin, the rate of Grade 3/4 neutropenia is 27%; the rate of Grade 3/4 anemia is 10%; and the rate of Grade 3/4 thrombocytopenia is 13%. With pemetrexed/cisplatin, the rate of Grade 3/4 neutopenia is 15%; the rate of Grade 3/4 anemia is 6%; and the rate of Grade 3/4 thrombocytopenia is 4%. (Scagliotti G V, et al. 2008.)

Non-hematologic adverse events vary according to the specific platinum doublet. For example, those related to gemcitabine/cisplatin include alopecia (21%, all grades), vomiting (6%, Grade 3/4), fatigue (5%, Grade 3/4), and febrile neutropenia (4%, Grade 3/4).25 Common non-hematologic adverse events associated with paclitaxel/carboplatin include neuropathy (18%, Grade 2/3), arthralgia (6%, Grade 3/4), fatigue (5%, Grade 3/4), and febrile neutropenia (3%, Grade 3/4). (Belani C P et al. J Clin Oncol 2008; 26: 468-73.) Besides histology, the choice of platinum doublet for any individual NSCLC patient may depend on the toxicities associated with different doublets.

### Cisplatin

Cisplatin is a platinum-based drug that is used in NSCLC. Cisplatin is administered intravenously at a dose of 75 mg/m² over 30 to 120 minutes after gemcitabine or over 120 minutes after pemetrexed. Subjects who are receiving cisplatin must be monitored for nephrotoxicity, ototoxicity and neuropathy in addition to myelosuppression. Caution must be observed in cases of nausea, vomiting and dehydration.

### Carboplatin

Carboplatin is a platinum-based drug that is used in combination with a taxane, gemcitabine, or pemetrexed for treatment of NSCLC. Carboplatin is administered intravenously at a dose of AUC 6 mg/mL*min (per Calvert formula) over 15 to 30 minutes after the use of paclitaxel or pemetrexed (Patel J D, et al. J Clin Oncol 2013; 34: 4349-57). Carboplatin may also be given at a dose of AUC 5 mg/mL*min (per Calvert formula) with gemcitabine. (Rosell R, et al. Lancet Oncol 2012; 13:239-46.) Subjects who are receiving carboplatin must be monitored for myelosuppression and anaphylaxis.

### Gemcitabine

Gemcitabine is indicated in combination with cisplatin in first-line treatment of inoperable, locally advanced (Stages IIIA or IIIB) or metastatic (Stage IV) NSCLC. Using the three week schedule, gemcitabine is administered intravenously at a dose of 1,250 mg/m² over 30 minutes on Days 1 and 8 of each 21-day cycle. Cisplatin should be administered 30 minutes after gemcitabine on Day 1 only at a dose of 75 mg/m².

Gemcitabine may also be given at a dose of 1000 mg/m² over 30 minutes on Days 1 and 8 for each 21-day cycle, in combination with carboplatin at AUC 5 mg/mL*min (per Calvert formula), as first-line treatment of advanced NSCLC.

Dose adjustments for hematologic toxicity may be required for gemcitabine and cisplatin (individually). Gemcitabine dosage adjustment for hematologic toxicities based upon the granulocyte and platelet counts on the day of treatment. Subjects receiving gemcitabine should be monitored prior to each dose using complete blood counts (CBC). If marrow suppression is noted, dose modifications can be made. For non-hematologic toxicities, other than alopecia and nausea, dose modifications should be considered for both gemcitabine and cisplatin.

### Pemetrexed

Pemetrexed is a folate analog metabolic inhibitor indicated as initial treatment for locally advanced or metastatic non-squamous NSCLC in combination with cisplatin. Pemetrexed is also indicated as maintenance treatment for locally advanced or metastatic non-squamous NSCLC patients whose disease has not progressed after platinum-based first-line chemotherapy. Pemetrexed is administered intravenously at a dose of 500 mg/m² on Day 1 of each 21-day cycle. Cisplatin can be administered 30 minutes after pemetrexed at a dose of 75 mg/m².

The pre-medication regimen for pemetrexed includes folic acid and vitamin B12 as well as dexamethasone or equivalent to reduce cutaneous reactions. Subjects receiving pemetrexed should be monitored prior to each dose using CBC and renal function tests. If marrow suppression is noted, dose modifications can be made. For renal toxicity, pemetrexed should be held when the creatinine clearance (CrCl)<45 mL/minute. Caution should be used among subjects who are receiving non-steroidal anti-inflammatory drugs (NSAIDs) and who have mild to moderate renal insufficiency (CrCl between 45 and 79 mL/minute). Caution should also be used when nephrotoxic drugs are administered with pemetrexed.

Nivolumab Combined with Ipilimumab

In CA209012, the combination of nivolumab and ipilimumab has been studied as first line therapy for subjects with previously untreated stage IV or recurrent NSCLC at several different dose and schedules. As of 17 Mar. 2015, 80 patients have been treated with this combination in the original 5 cohorts. An additional 111 patients have been treated in the newer 3 cohorts. At least one AE, regardless of causality, was reported in 100% of subjects treated in the original cohorts, and in the newer cohorts 80% of subjects have reported 1 AE. In the original cohorts, the most common (reported at >15% incidence) treatment related AEs (any Grade %; Grade 3-4%: 82; 43) are fatigue (40; 4), diarrhea (30; 6), rash (28; 8), decreased appetite (19; 0), lipase increased (15; 8), and nausea (15; 1). In the newer cohorts, the most common (reported at >10% incidence) treatment related AEs (any Grade %; Grade 3-4%: 59; 26) are diarrhea (14; 3), rash (14; 4), and fatigue (12; 1).

In the original cohorts, the majority of AEs leading to discontinuation (regardless of causality) were Grade 3 or 4 (reported in 17 of 80 subjects, 21%). Grade 3 events included pneumonitis, ALT increased, AST increased, colitis, diarrhea, ulcerative colitis, delayed gastric emptying, Miller Fisher syndrome, allergic nephritis, and rash. One subject discontinued due to Grade 4 event of ALT increased, and 2 subjects discontinued for AST increased. One patient died from pulmonary hemorrhage.

In the “1+1” cohort, the most common (reported at >10% incidence) treatment related AEs (any Grade %; Grade 3-4%) were fatigue (29; 0), rash (29; 13), diarrhea (19; 0), lipase increased (13; 7), pruritus (16; 0). Only four patients (13%) in this cohort reported AEs leading to discontinuation of study treatment: pneumonitis (1), AST increased (1), myalgia (1), and rash (1). There were no treatment-related deaths.

Efficacy data from this cohort indicates a substantial level of clinical activity. At a median follow-up of 50 weeks, the median overall survival has not been reached. The median PFS is 34 weeks (8.5 months) compared to approximately 4-5 months for platinum-based chemotherapy. This is despite the lower overall response rate of 16%, compared to 25-30% observed with platinum-based chemotherapy. Activity is observed in both PD-L1+ and PD-L1− tumors.

In the newer cohorts, the most common (reported at >10% incidence) treatment related AEs (any Grade %; Grade 3-4%) are diarrhea (14; 3), rash (14; 4), and fatigue (12; 1). Eight patients (7%) reported AES leading to discontinuation of study treatment: autoimmune hepatitis (2) and 1 each of colitis, encephalopathy, facial nerve disorder, infusion related reaction, pneumonitis, rash, and transaminases increased. There have been no treatment related deaths.

Nivolumab 3 mg/kg every 2 weeks appears to be the most active nivolumab dose in previously treated subjects with NSCLC (Brahmer J R, et al. Nivolumab (anti-PD-1; BMS-936558; ONO-4538) in patients with non-small cell lung cancer (NSCLC): Overall survival and long-term safety in a phase 1 trial. Presented at World Conference on Lung Cancer, 2013) and it is the dose being evaluated in CA209026, the open-label study of nivolumab monotherapy compared to Investigator's Choice chemotherapy as first-line therapy for stage IV or recurrent PD-L1+ NSCLC. Thus, for CA209227 the second nivolumab/ipilimumab dosing schedule will use this dose of nivolumab, combined with ipilimumab 1 mg/kg, every 6 weeks. It is postulated that the safety of the combination may be enhanced by administering the ipilimumab at less frequent intervals, and the efficacy enhanced by continuous administration during the course of therapy, instead of 4 induction doses only.

### Overall Risk/Benefit Assessment

Subjects with newly diagnosed metastatic or recurrent NSCLC represent a great unmet need. Nivolumab and nivolumab plus ipilimumab in NSCLC have potential for improved clinical outcomes. Preliminary data suggest that PD-L1 negative tumors, as well as PD-L1 positive tumors, may respond to nivolumab or the combination of nivolumab plus ipilimumab. The benefit of nivolumab or combination immunotherapy with nivolumab plus ipilimumab over standard-of-care platinum-based first-line chemotherapy is being tested in CA209227. The platinum-based chemotherapy regimens have similar clinical activity and well described safety profiles, characterized by myelosuppression and other regimen-specific non-hematologic toxicities, such as peripheral neuropathy, nausea/vomiting, and renal impairment. The safety profile of nivolumab and nivolumab plus ipilimumab is characterized by immune-related toxicities, such as diarrhea, rash, pneumonitis, liver toxicity, and endocrinopathies. The frequencies and intensities of these events in the combination are variable and depend on the specific doses and schedule used. In the two dosing schedules selected, these events were mostly low grade and manageable with the use of corticosteroids.

In order to assess the potential benefit of nivolumab monotherapy and nivolumab plus ipilimumab in the treatment of patients with PD-L1 positive and negative advanced NSCLC compared to standard-of-care platinum-based first-line chemotherapy, and to assess the contribution of ipilimumab to treatment regimen, a randomized trial comparing nivolumab and nivolumab plus ipilimumab to platinum doublet chemotherapy in subjects with stage IV or recurrent PD-L1 positive and negative NSCLC is performed as described herein.

### Study Design and Duration

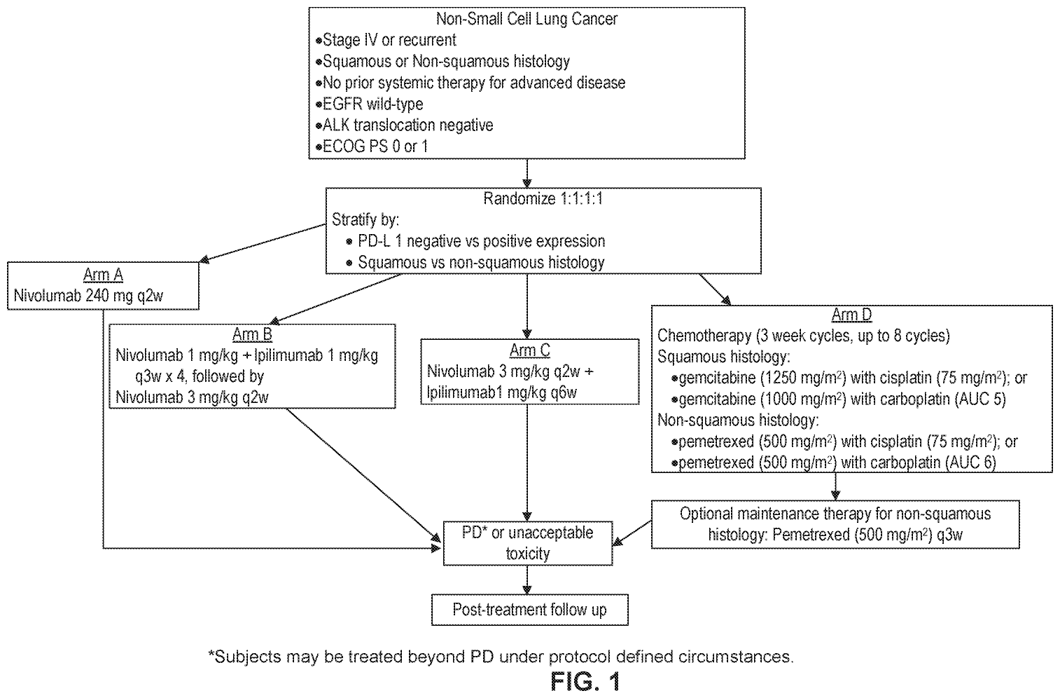

The study design schematic is presented in FIG. 1.

The screening phase begins by establishing the subject's initial eligibility and signing of the informed consent (ICF). A subject is enrolled using the Interactive Voice Response System (IVRS). Tumor tissue (archival or recent tumor biopsy) is submitted for determination of PD-L1 status. A subject is assessed for study eligibility. All screening assessments and procedures must be performed within 28 days prior to randomization.

The treatment phase begins when the randomization is made. The subject is randomly assigned to one of the four treatment arms. Study treatment must begin within 3 working days of randomization.

The duration of the study from start of enrollment to analysis of the primary OS endpoint is expected to be approximately 48 months. The study will end when analysis of survival is complete.

At the conclusion of the study, subjects who continue to demonstrate clinical benefit will be eligible to receive study drug (nivolumab and/or ipilimumab) up to 12 months after the approval of study drug by the responsible health authority, or until the study drug becomes commercially available within the country, whichever occurs sooner. Study drug (nivolumab and/or ipilimumab) will be provided via an extension of the study, a rollover study requiring approval by responsible health authorities and ethics committee, or through another mechanism.

### Target Population

The target population inclusion criteria include:

a) ECOG Performance Status of greater than or equal to 1 (See Table 7);

TABLE 7
ECOG PERFORMANCE STATUS^{a}

| | |
|---|---|
| 0 | Fully active, able to carry on all pre-disease performance |
| | without |
| 1 | Restricted in physically strenuous activity but ambulatory |
| | and able to carry out work of a light or sedentary nature, |
| | e.g., light house work, office work |
| 2 | Ambulatory and capable of all selfcare but unable to carry |
| | out any work activities. Up and about more than 50% of |
| | waking hours |
| 3 | Capable of only limited selfcare, confined to bed or chair |
| | more than 50% of waking hours |
| 4 | Completely disabled. Cannot carry on any selfcare. Totally |
| | confined to bed or chair |
| 5 | Dead |

^{a}Oken M M, Creech R H, Tormey D C, Horton J, Davis T E, McFadden E T, and Carbone P P. Toxicity and Response Criteria of the Eastern Cooperative Oncology Group. Am J Clin Oncol 1982; 5: 649-655

b) Subjects with histologically confirmed Stage IV or recurrent NSCLC (per the 7th International Association for the Study of Lung Cancer classification (IASLC)) squamous or non-squamous histology, with no prior systemic anti-cancer therapy (including EGFR and ALK inhibitors) given as primary therapy for advanced or metastatic disease. Prior adjuvant or neoadjuvant chemotherapy is permitted as long as the last administration of the prior regimen occurred at least 6 months prior to enrollment. Prior definitive chemoradiation for locally advanced disease is also permitted as long as the last administration of chemotherapy or radiotherapy (which ever was given last) occurred at least 6 months prior to enrollment;

c) Measurable disease by CT or MRI per RECIST 1.1 criteria and radiographic tumor assessment performed within 28 days of randomization. Target lesions may be located in a previously irradiated field if there is documented (radiographic) disease progression in that site after the completion of radiation therapy;

d) Subjects will have PD-L1 IHC testing. Either a formalin-fixed, paraffin-embedded (FFPE) tissue block or unstained tumor tissue sections, with an associated pathology report, must be submitted for biomarker evaluation prior to randomization. The tumor tissue sample may be fresh or archival if obtained within 6 months prior to enrollment, and there can have been no systemic therapy (e.g., adjuvant or neoadjuvant chemotherapy) given after the sample was obtained. Tissue can be a core needle biopsy, excisional or incisional biopsy. Fine needle biopsies or drainage of pleural effusions with cytospins are not considered adequate for biomarker review and randomization. Biopsies of bone lesions that do not have a soft tissue component or decalcified bone tumor samples are also not acceptable;

e) Prior palliative radiotherapy to non-CNS lesions must have been completed at least 2 weeks prior to randomization. Subjects with symptomatic tumor lesions at baseline that may require palliative radiotherapy within 4 weeks of randomization are strongly encouraged to receive palliative radiotherapy prior to randomization;

Screening laboratory values must meet the following criteria (using CTCAE v4): i) WBC greater than or equal to 2000 ii) Neutrophils greater than or equal to 1500/μL, iii) Platelets greater than or equal to 100×10³/μL, iv) Hemoglobin greater than or equal to 9.0 g/dL, v) Serum creatinine less than or equal to 1.5×ULN or calculated creatinine clearance greater than or equal to 50 mL/min (using the Cockcroft Gault formula) [Female CrCl=((140-age in years)×weight in kg×0.85)/(72×serum creatinine in mg/dL)] and [Male CrCl=((140-age in years)×weight in kg×1.00)/(72×serum creatinine in mg/dL)], vi) AST/ALTless than or equal to 3.0×ULN, vii) Total bilirubin less than or equal to 1.5×ULN except subjects with Gilbert Syndrome who must have a total bilirubin level <3.0 mg/dL).

This study permits the re-enrollment of a subject who has discontinued the study as a pre-treatment failure (ie, subject has not been randomized/has not been treated).

### Age and Reproductive Status

The age and reproductive status criteria include a) Males and Females, ages greater than or equal to 18 years of age; b) Women of childbearing potential (WOCBP) must have a negative serum or urine pregnancy test (minimum sensitivity 25 IU/L or equivalent units of HCG) within 24 hours prior to the start of study drug; c) Women must not be breastfeeding; d) WOCBP must agree to follow instructions for method(s) of contraception from the time of enrollment for the duration of treatment plus 5 half-lives of nivolumab (half-life up to 25 days) plus 30 days (duration of ovulatory cycle) for a total of 23 weeks post treatment completion (for subjects treated in arms A, B and C); e) Men who are sexually active with WOCBP must agree to follow instructions for method(s) of contraception for the duration of treatment with nivolumab plus 5 half-lives of nivolumab plus 90 days (duration of sperm turnover) for a total of 31 weeks post treatment completion (for subjects treated in arms A, B and C).

### Exclusion Criteria

Target Disease Exceptions:

a) Subjects with known EGFR mutations which are sensitive to available targeted inhibitor therapy (including, but not limited to, deletions in exon 19 and exon 21 [L858R] substitution mutations) are excluded. All subjects with non-squamous histology must have been tested for EGFR mutation status. Subjects with non-squamous histology and unknown or indeterminate EGFR status are excluded.

b) Subjects with known ALK translocations which are sensitive to available targeted inhibitor therapy are excluded. If tested, use of an FDA-approved test is strongly encouraged. Subjects with unknown or indeterminate ALK status may be enrolled.

c) Subjects with untreated CNS metastases are excluded. Subjects are eligible if CNS metastases are adequately treated and subjects are neurologically returned to baseline (except for residual signs or symptoms related to the CNS treatment) for at least 2 weeks prior to randomization. In addition, subjects must be either off corticosteroids, or on a stable or decreasing dose of less than or equal to 10 mg daily prednisone (or equivalent) for at least 2 weeks prior to randomization.

d) Subjects with carcinomatous meningitis.

Medical History and Concurrent Diseases:

a) Subjects must have recovered from the effects of major surgery or significant traumatic injury at least 14 days before randomization.

b) Subjects with previous malignancies (except non-melanoma skin cancers, and in situ cancers such as the following: bladder, gastric, colon, cervical/dysplasia, melanoma, or breast) are excluded unless a complete remission was achieved at least 2 years prior to randomization and no additional therapy is required or anticipated to be required during the study period.

c) Other active malignancy requiring concurrent intervention.

d) Subjects with an active, known or suspected autoimmune disease. Subjects with type I diabetes mellitus, hypothyroidism only requiring hormone replacement, skin disorders (such as vitiligo, psoriasis, or alopecia) not requiring systemic treatment, or conditions not expected to recur in the absence of an external trigger are permitted to enroll.

e) Subjects with a condition requiring systemic treatment with either corticosteroids (>10 mg daily prednisone equivalent) or other immunosuppressive medications within 14 days of randomization. Inhaled or topical steroids, and adrenal replacement steroid >10 mg daily prednisone equivalent, are permitted in the absence of active autoimmune disease.

Subjects with interstitial lung disease that is symptomatic or may interfere with the detection or management of suspected drug-related pulmonary toxicity.

g) Known history of testing positive for human immunodeficiency virus (HIV) or known acquired immunodeficiency syndrome (AIDS).

Physical and Laboratory Test Findings:

a) Any positive test for hepatitis B virus or hepatitis C virus indicating acute or chronic infection, or b) Subjects with greater than or equal to Grade 2 peripheral neuropathy.

Allergies and Adverse Drug Reaction:

History of allergy or hypersensitivity to platinum-containing compounds or other study drug components

Prohibited and/or Restricted Treatments:

The following strong CYP3A4 inhibitors should be avoided during the study. This includes, but not is limited to, the following: Ketoconazole, Itraconazole, Clarithromycin, Nefazodone, Teithromycin, and Voriconzole.

The following medications are prohibited during the study (unless utilized to treat a drug related adverse event): Immunosuppressive agents; Immunosuppressive doses of systemic corticosteroids (except as elsewhere in this application); Any concurrent anti-neoplastic therapy (i.e., chemotherapy, hormonal therapy, immunotherapy, extensive, non-palliative radiation therapy, or standard or investigational agents for treatment of NSCLC).

Other Restrictions and Precautions:

Subjects with a condition requiring systemic treatment with either corticosteroids (>10 mg daily prednisone equivalent) or other immunosuppressive medications within 14 days of randomization are excluded. Inhaled or topical steroids, and adrenal replacement steroid doses >10 mg daily prednisone equivalent, are permitted in the absence of active autoimmune disease.

Subjects with renal insufficiency should be assessed as to whether or not they should receive contrast and if so, what type and dose of contrast is appropriate. Specific to MRI, subjects with severe renal insufficiency (i.e., estimated glomerular filtration rate (eGFR)<30 mL/min/1.73 m²) are at increased risk of nephrogenic systemic fibrosis. MRI contrast should not be given to this subject population. In addition, subjects are excluded from MM if they have tattoos, metallic implants, pacemakers, etc. The ultimate decision to perform MRI in an individual subject in this study rests with the site radiologist, the investigator and the standard set by the local Ethics Committee.

Permitted Therapy:

Subjects are permitted the use of topical, ocular, intra-articular, intranasal, and inhalational corticosteroids (with minimal systemic absorption). Adrenal replacement steroid doses >10 mg daily prednisone are permitted. A brief (less than 3 weeks) course of corticosteroids for prophylaxis (e.g., contrast dye allergy) or for treatment of non-autoimmune conditions (e.g., delayed-type hypersensitivity reaction caused by a contact allergen) is permitted.

Concomitant palliative and supportive care for disease related symptoms (including bisphosphonates and RANK-L inhibitors) is allowed if initiated prior to first dose of study therapy. Prior palliative radiotherapy must have been completed at least 2 weeks prior to randomization.

Palliative Local Therapy:

Palliative local therapy, including palliative radiation therapy and palliative surgical resection, to symptomatic non-target bone lesions, skin lesions, or CNS lesions is permitted prior to discontinuation of study treatment for subjects who do not have evidence of overall clinical or radiographic progression per RECIST 1.1. Palliative local therapy to lesions causing hemoptysis may also be permitted prior to discontinuation of study treatment in subjects who do not have evidence of overall clinical or radiographic progression per RECIST 1.1, provided that the lesions undergoing palliative local therapy are not the only sites of measurable disease and the case is discussed with and approved by the Medical Monitor.

The potential for overlapping toxicities with radiotherapy and nivolumab/ipilimumab currently is not known; however, anecdotal data suggests that it is tolerable. As concurrent radiotherapy and nivolumab/ipilimumab have not been formally evaluated, in cases where palliative radiotherapy is required for a tumor lesion, then nivolumab/ipilimumab should be withheld for at least 1 week before, during, and 1 week after radiation. Subjects should be closely monitored for any potential toxicity during and after receiving radiotherapy, and AEs should resolve to Grade less than or equal to 1 prior to resuming nivolumab.

### Post Study Drug Study Follow Up

In this study, overall survival is a key endpoint. Post study follow-up is of critical importance and is essential to preserving subject safety and the integrity of the study. Follow-Up Visit 1 to occur 35 days from the last dose (+/−7 days) or coinciding with the date of discontinuation of study drug (+/−7 days) if the date of discontinuation is greater than 42 days from the last dose. Follow-Up Visit 2 to occur 80 days from Follow-Up Visit 1 (+/−7 days). Survival Follow-Up Visits to occur approximately every 3 months from Follow-Up Visit 2.

### Study Drugs for CA209227

The drug products used for the study described herein are shown in Table 8.

TABLE 8
Product Description - Treatment Phase

| | | Primary Packaging | Secondary | | |
|---|---|---|---|---|---|
| Product Description | | (Volume)/Label | Packaging | | Storage Conditions |
| and Dosage Form | Potency | [symbol] | [symbol] | Appearance | (per label) |
| BMS-936558-01 | 100 mg | 10 mL per vial/ | 5 or 10 vials per | Clear to opalescent colorless | 2 to 8° C. Protect from |
| Solution for Injection^{a} | (10 mg/mL) | Open-label | carton/Open-label | to pale yellow liquid. May | light and freezing |
| | | | | contain particles | |
| Ipilimumab Solution | 200 mg | 40 mL vial/ | 4 vials per | Clear to opalescent colorless | 2 to 8° C. Protect from |
| for Injection | (5 mg/mL) | Open-label | carton/Open-label | to pale yellow liquid. May | light and freezing |
| | | | | contain particles | |
| Carboplatin Solution | 450 mg/vial | 45 mL per vial/ | 4 vials per | Clear, colorless or slightly | Store at or below 25° C. |
| for Injection^{b} | (10 mg/mL) | Open label | carton/Open-label | yellow solution | Protect from light |
| Cisplatin Concentrate | 100 mg/vial | 100 mL per vial/ | 4 vials per | Clear, colorless solution | Do not store above |
| for Solution for | (1 mg/mL) | Open-label | carton Open-label | | 25° C. Do not |
| Infusion | | | | | refrigerate or freeze. |
| | | | | | Store in original |
| | | | | | container. |
| Gemcitabine Powder | 1000 mg/vial | 1000 mg per vial/ | 1 vial per | White to off-white plug or | Store at 15-30° C. |
| for Solution for | | Open label | carton/Open label | powder | |
| Infusion^{b} | | | | | |
| Gemcitabine Solution | 1000 mg/vial | 1000 mg per vial/ | 1 vial per | Clear, colorless or light | Store at 2-8° C. |
| for Infusion^{b} | | Open label | carton/Open label | straw-colored solution | |
| Pemetrexed Powder for | 500 mg/vial | 500 mg per vial/ | 1 vial per | White to either light yellow | Store at 25° C. |
| Concentrate for | | Open label | carton/Open- label | or green-yellow lyophilised | Excursions permitted |
| Solution for Infusion^{b} | | | | powder | (15-30° C.) |

^{a}May be labeled as either “BMS-936558-01” or “Nivolumab”.
^{b}These products may be obtained by the investigational sites as local commercial product in certain countries if allowed by local regulations. In these cases, products may be a different pack size/potency than listed in the table. These products should be prepared/stored/administered in accordance with the package insert or summary of product characteristics (SmPC). [symbol]
indicates data missing or illegible when filed

In the CA209227 protocol, the investigational products are: BMS-936558 (nivolumab), Ipilimumab, Gemcitabine, Cisplatin, Carboplatin, and Pemetrexed.

### Storage and Dispensing

Nivolumab is to be administered as an approximately 30 minute IV infusion in the 3 nivolumab containing arms. At the end of the infusion, flush the line with a sufficient quantity of normal saline or dextrose solution. Ipilimumab is to be administered as an approximately 30 minute IV infusion. At the end of the infusion, flush the line with a sufficient quantity of normal saline or 5% dextrose solution. When both study drugs are to be administered on the same day, separate infusion bags and filters must be used for each infusion. Nivolumab is to be administered first. The nivolumab infusion must be promptly followed by a saline flush to clear the line of nivolumab before starting the infusion. The second infusion will always be ipilimumab, and will start at least 30 minutes after completion of the nivolumab infusion.

### Selection and Timing of Dose for Each Subject

A dosing schedule is detailed in Table 16 (Example 1 below). 103231 Arm A Dosing (nivolumab monotherapy): Subjects randomized to Arm A will receive treatment with nivolumab at a dose of 240 mg as a 30 minute IV infusion, on Day 1 of each treatment cycle every 2 weeks, until progression, unacceptable toxicity, withdrawal of consent, or the study ends, whichever occurs first.

Arm B Dosing (nivolumab plus ipilimumab): Subjects randomized to Arm B will receive treatment with nivolumab 1 mg/kg as a 30 minute infusion and ipilimumab 1 mg/kg as a 30 minute infusion on Day 1 of each treatment cycle every 3 weeks for 4 cycles, followed by nivolumab 3 mg/kg as a 30 minute infusion every 2 weeks. Treatment will continue until progression, unacceptable toxicity, withdrawal of consent, or the study ends, whichever occurs first. When nivolumab and ipilimumab are to be administered on the same day, separate infusion bags and filters must be used for each infusion. Nivolumab is to be administered first. The second infusion will always be ipilimumab and will start no sooner than 30 minutes after completion of the nivolumab infusion. Nivolumab and ipilimumab may be diluted in 0.9% Sodium Chloride Solution or 5% Dextrose solution.

Dosing calculations should be based on the body weight. It is not necessary, but may be standard of care, to re-calculate the dose if the subject weight is within 10% of the weight used to calculate the previous dose. All doses should be rounded to the nearest milligram. No dose modifications are allowed. Subjects can be dosed no less than 12 days from the previous dose. There are no pre-medications recommended. Doses of nivolumab and/or ipilimumab may be interrupted, delayed, or discontinued depending on how well the subject tolerates the treatment.

Arm C Dosing (nivolumab plus ipilimumab): Subjects randomized to Arm C will receive treatment with nivolumab as a 30 minute infusion 3 mg/kg every 2 weeks and ipilimumab as a 30 minute infusion 1 mg/kg every 6 weeks, starting on Day 1, until progression, unacceptable toxicity, withdrawal of consent, or the study ends, whichever occurs first. When nivolumab and ipilimumab are to be administered on the same day, separate infusion bags and filters must be used for each infusion. Nivolumab is to be administered first. The second infusion will always be ipilimumab and will start no sooner than 30 minutes after completion of the nivolumab infusion. Nivolumab and ipilimumab may be diluted in 0.9% sodium chloride solution or 5% dextrose solution.

Dosing calculations should be based on the body weight. If the subject's weight on the day of dosing differs by >10% from the weight used to calculate the prior dose, the dose must be recalculated. All doses should be rounded to the nearest milligram. No dose modifications are allowed. Subjects can be dosed with nivolumab no less than 12 days from the previous dose. There are no pre-medications recommended. Doses of nivolumab and/or ipilimumab may be interrupted, delayed, or discontinued depending on how well the subject tolerates the treatment.

Arm D Dosing (Platinum Doublet Chemotherapy):

Squamous Histology Chemotherapy Options

Subjects with squamous histology who are randomized to Arm D may receive either of the following gemcitabine/platinum regimens:

1) Gemcitabine/Cisplatin: Subjects will receive gemcitabine at a dose of 1250 mg/m² as a 30 minute IV infusion on days 1 and 8 with cisplatin at a dose of 75 mg/m² as a 30 to 120 minute IV infusion on Day 1 of a 3-week treatment cycle for up to 6 cycles. At the discretion of the investigator and according to local standards of care, gemcitabine/cisplatin may also be discontinued after cycle 4 in subjects whose disease is felt by the investigator not likely to benefit from additional platinum doublet chemotherapy. Dosing calculations should be based on the body surface area calculation. The dose may remain the same if the subject's weight is within 10% of the baseline weight or prior dose weight.

Cisplatin will be administered to patients following the end of the gemcitabine infusion. Pretreatment hydration for cisplatin can follow local standard of care, or 1 to 2 liters of fluid (per local standards) infused IV for 8 to 12 hours prior to cisplatin infusion is recommended. Adequate hydration and urinary output must be maintained for at least 24 hours following cisplatin administration. Administration and monitoring should be performed according to local standards. Use of mannitol following the cisplatin infusion should also follow local standards-of-care.

Pre-medications: Antiemetic pre-medication will be administered according to local standards. Recommended antiemetic treatments are dexamethasone (dosing according to local standards; an equivalent dose of another corticosteroid may be substituted) and a 5-HT3 receptor antagonist (type per investigator discretion and local standards-of-care). Additional use of antiemetic pre-medications may be employed at the discretion of the Investigator.

Doses of gemcitabine and/or cisplatin may be interrupted, delayed, reduced, or discontinued depending on how well the subject tolerates the treatment.

All subjects who will be receiving cisplatin should have audiometric testing performed prior to initiation of therapy and prior to subsequent doses of cisplatin, or as per local standards of care.

Subjects who discontinue cisplatin alone may, at the investigator's discretion, be switched to gemcitabine/carboplatin for the remainder of the platinum doublet cycles (up to 6 cycles in total). Dosing for gemcitabine/carboplatin for such subjects should follow the dosing in the Gemcitabine/Carboplatin section here.

2) Gemcitabine/Carboplatin: Subjects will receive gemcitabine at a dose of 1000 mg/m² as a 30 minute IV infusion on Days 1 and 8 with carboplatin at a dose of AUC 5 as a 30 minute IV infusion, on Day 1 of a 3-week cycle, for up to 6 cycles. At the discretion of the investigator and according to local standards of care, gemcitabine/carboplatin may also be discontinued after cycle 4 in subjects whose disease is felt by the investigator not likely to benefit from additional platinum doublet chemotherapy. Gemcitabine dosing calculations should be based on the body surface area calculation. The dose may remain the same if the subject's weight is within 10% of the baseline weight or prior dose weight.

Carboplatin should be given following gemcitabine on Day 1 of each cycle, and the carboplatin dose will be calculated using the Calvert formula as follows: Carboplatin dose (mg)=Target AUC×[(CrCl (ml/min)+25]; Creatinine clearance (CrCl) calculation is based on the Cockroft-Gault formula) and should include the most recent serum creatinine and most recent weight. If calculation of the CrCl by the Cockroft-Gault formula yields a result of >125 mL/min, then a CrCl should be calculated by an alternative formula per institutional standards or capped at 125 mL/min.

Pre-medications: Oral antiemetic pre-medication will be administered according to local standards. Recommended antiemetic treatments are dexamethasone (dosing according to local standards; an equivalent dose of another corticosteroid may be substituted) and a 5-HT3 receptor antagonist (type per investigator discretion and local standards of care). Additional use of antiemetic pre-medications may be employed at the discretion of the investigator per local standards of care.

Doses of gemcitabine and/or carboplatin may be interrupted, delayed, reduced, or discontinued depending on how well the subject tolerates the treatment.

Non-Squamous Histology Chemotherapy Options

Subjects with non-squamous histology who are randomized to Arm D may receive one of the following pemetrexed/platinum regimens, with or without pemetrexed continuation maintenance therapy:

1) Pemetrexed/Cisplatin With or Without Pemetrexed Continuation Maintenance: Subjects will receive pemetrexed at a dose of 500 mg/m² as a 10 minute IV infusion on Day 1 with cisplatin at a dose of 75 mg/m² as a 120 minute IV infusion on Day 1 of a 3-week treatment cycle, for up to 6 cycles. At the discretion of the investigator and according to local standards of care, pemetrexed/cisplatin may also be discontinued after cycle 4 in subjects whose disease is felt by the investigator not likely to benefit from additional platinum doublet chemotherapy. After cycle 4, subjects with stable disease or response may also discontinue cisplatin and continue pemetrexed at the same dose and schedule as continuation maintenance until progression, unacceptable toxicity, or withdrawal of consent. In subjects who required pemetrexed dose reduction due to toxicity during the pemetrexed/cisplatin combination cycles, the dose of pemetrexed may be escalated to 500 mg/m² after the discontinuation of cisplatin, at the investigator's discretion and according to local standards, if the prior toxicity was felt to be related mainly to cisplatin. Dosing calculations should be based on the body surface area calculation and may be capped per local standards. The dose may remain the same if the subject's weight is within 10% of the baseline weight or prior dose weight.

Cisplatin will be administered to subjects at least 30 minutes following the end of the pemetrexed infusion. Pretreatment hydration for cisplatin can follow local standard of care, or use 1 to 2 liters of fluid (per local standards) infused IV for 8 to 12 hours prior to cisplatin infusion is recommended. Adequate hydration and urinary output must be maintained for at least 24 hours following cisplatin administration. Administration and monitoring should be performed according to local standards. Use of mannitol following the cisplatin infusion should also follow local standards-of-care.

Pre-medications for use with pemetrexed: Oral corticosteroid should be given according to local standards at a dose equivalent to dexamethasone 4 mg BID on the day prior to, the day of, and the day after the administration of pemetrexed. Oral folic acid 350 to 1000 mcg daily should be given starting 1 week prior to the first dose of pemetrexed, with at least 5 doses of folic acid administered in the 7 days prior to the first dose. Oral folic acid should be continued daily throughout the treatment with pemetrexed and for 21 days after the last dose of pemetrexed. Intramuscular (IM) injection of vitamin B12 1000 mcg should be given approximately one week prior to the first dose of pemetrexed repeated every 3 cycles thereafter during pemetrexed treatment. Subsequent injections of vitamin B12 may be given on the same day as pemetrexed. (Subjects with non-squamous histology may begin folic acid and vitamin B12 prior to randomization in anticipation of pemetrexed should they be randomized to Arm D.)

Pre-medications for use with pemetrexed/cisplatin: Antiemetic pre-medication will be administered according to local standards. Recommended antiemetic treatments are dexamethasone (dosing according to local standards; an equivalent dose of another corticosteroid may be substituted) and a 5-HT3 receptor antagonist (type per investigator discretion and local standards-of-care). Additional use of antiemetic pre-medications may be employed at the discretion of the Investigator.

Doses of pemetrexed and/or cisplatin may be interrupted, delayed, reduced, or discontinued depending on how well the subject tolerates the treatment

All subjects who will be receiving cisplatin should have audiometric testing performed prior to initiation of therapy and prior to subsequent doses of cisplatin, or as per local standards of care.

Subjects who discontinue cisplatin alone may, at the investigator's discretion, be switched to pemetrexed/carboplatin for the remainder of the platinum doublet cycles (up to 6 cycles in total). Dosing for pemetrexed/carboplatin for such subjects should follow the instructions in the Pemetrexed/Carboplatin With or Without Pemetrexed Continuation Maintenance section below.

2) Pemetrexed/Carboplatin With or Without Pemetrexed Continuation Maintenance: Subjects will receive pemetrexed at a dose of 500 mg/m² as a 10 minute IV infusion on Day 1, followed by carboplatin at a dose of AUC 6 as a 30 minute IV infusion, on Day 1 of a 3-week treatment cycle, for up to 6 cycles.

At the discretion of the investigator and according to local standards of care, pemetrexed/carboplatin may also be discontinued after cycle 4 in subjects whose disease is felt by the investigator not likely to benefit from additional platinum doublet chemotherapy.

After cycle 4, subjects with stable disease or response may also discontinue carboplatin and continue pemetrexed at the same dose and schedule as continuation maintenance until progression, unacceptable toxicity, or withdrawal of consent. In subjects who required pemetrexed dose reduction due to toxicity during the pemetrexed/carboplatin combination cycles, the dose of pemetrexed may be escalated to 500 mg/m² after the discontinuation of carboplatin, at the investigator's discretion and according to local standards, if the prior toxicity was felt to be related mainly to carboplatin.

Pemetrexed dosing calculations should be based on the body surface area calculation. The dose may remain the same if the subject's weight is within 10% weight used to calculate the previous dose.

The carboplatin dose will be calculated using the Calvert formula as follows: Carboplatin dose (mg)=Target AUC×[(CrCl (ml/min)+25]; Creatinine clearance (CrCl) calculation is based on the Cockroft-Gault formula and should include the most recent serum creatinine and most recent weight. If calculation of the CrCl by the Cockroft-Gault formula yields a result of >125 mL/min, then a CrCl should be calculated by an alternative formula per institutional standards or capped at 125 mL/min.

Pre-medication for use with pemetrexed: Oral corticosteroid should be given according to local standards at a dose equivalent to dexamethasone 4 mg BID on the day prior to, the day of, and the day after the administration of pemetrexed. Oral folic acid 350 to 1000 mcg daily should be given starting 1 week prior to the first dose of pemetrexed, with at least 5 doses of folic acid administered in the 7 days prior to the first dose. Oral folic acid should be continued daily throughout the treatment with pemetrexed and for 21 days after the last dose of pemetrexed. Intramuscular (IM) injection of vitamin B12 1000 mcg should be given approximately one week prior to the first dose of pemetrexed and repeated every 3 cycles thereafter during pemetrexed treatment. Subsequent injections of vitamin B12 may be given on the same day as pemetrexed. (Subjects with non-squamous histology may begin folic acid and vitamin B12 prior to randomization in anticipation of pemetrexed should they be randomized to Arm D.)

Pre-medications for use with pemetrexed/carboplatin: Antiemetic pre-medication will be administered according to local standards. Recommended antiemetic treatments are dexamethasone (dosing according to local standards; an equivalent dose of another corticosteroid may be substituted) and a 5-HT3 receptor antagonist (type per investigator discretion and local standards-of-care). Additional use of antiemetic pre-medications may be employed at the discretion of the Investigator.

Doses of pemetrexed and/or carboplatin may be interrupted, delayed, reduced, or discontinued depending on how well the subject tolerates the treatment.

### Dose Delay for Arm A (Nivolumab Monotherapy)

Nivolumab administration should be delayed for the following:

Any Grade greater than or equal to 2 non-skin, drug-related adverse event, except for fatigue and laboratory abnormalities; Any Grade greater than or equal to 3 skin drug-related AE; Any Grade 3 drug-related laboratory abnormality with the following exceptions for lymphopenia, AST, ALT, or total bilirubin or asymptomatic amylase or lipase: Grade 3 lymphopenia does not require a dose delay. If a subject has a baseline AST, ALT, or total bilirubin that is within normal limits, delay dosing for drug-related Grade greater than or equal to 2 toxicity. If a subject has baseline AST, ALT, or total bilirubin within the Grade 1 toxicity range, delay dosing for drug-related Grade greater than or equal to 3 toxicity, Any Grade greater than or equal to 3 drug-related amylase or lipase abnormality that is not associated with symptoms or clinical manifestations of pancreatitis does not require dose delay. The BMS Medical Monitor should be consulted for such Grade greater than or equal to 3 amylase or lipase abnormalities; and Any AE, laboratory abnormality or inter-current illness which, in the judgment of the investigator, warrants delaying the dose of study medication.

### Dose Delay for Arms B and C (Nivolumab Plus Ipilimumab)

Nivolumab and ipilimumab administration should be delayed for the following: Any Grade greater than or equal to 2 non-skin, drug-related adverse event, except for fatigue and laboratory abnormalities; Any Grade greater than or equal to 3 skin drug-related AE; Any Grade greater than or equal to 3 drug-related laboratory abnormality with the following exceptions for lymphopenia, AST, ALT, or total bilirubin or asymptomatic amylase or lipase: Grade 3 lymphopenia does not require a dose delay. If a subject has a baseline AST, ALT, or total bilirubin that is within normal limits, delay dosing for drug-related Grade greater than or equal to 2 toxicity. If a subject has baseline AST, ALT, or total bilirubin within the Grade 1 toxicity range, delay dosing for drug-related Grade greater than or equal to 3 toxicity, Any Grade greater than or equal to 3 drug-related amylase or lipase abnormality that is not associated with symptoms or clinical manifestations of pancreatitis does not require dose delay. The BMS Medical Monitor should be consulted for such Grade greater than or equal to 3 amylase or lipase abnormalities; and Any AE, laboratory abnormality or inter-current illness which, in the judgment of the investigator, warrants delaying the dose of study medication.

Subjects receiving ipilimumab in combination with nivolumab that have drug-related toxicities that meet the criteria for dose delay, should have both drugs (ipilimumab and nivolumab) delayed until retreatment criteria are met. (Exceptions apply to the retreatment criteria after dose delay of ipilimumab and nivolumab for Grade greater than or equal to 3 amylase and lipase abnormalities that are not associated with symptoms or clinical manifestations of pancreatitis and that are attributed to ipilimumab alone.

In Arm C, nivolumab may be delayed until the next planned ipilimumab dose if the next ipilimumab dose is scheduled within the next 12 days. This will permit periodic ipilimumab dosing to be synchronized with nivolumab dosing.

In Arm C, ipilimumab should be dosed at the specified interval regardless of any delays in intervening nivolumab doses. However, in order to maintain periodic synchronized dosing of ipilimumab and nivolumab, the dosing days of nivolumab and ipilimumab may be adjusted within the permitted +/−5 day window, as long as consecutive nivolumab doses are given at least 12 days apart. Ipilimumab may be delayed beyond the 5 day window if needed to synchronize with the next nivolumab dose.

In Arm C, if an ipilimumab dose is delayed beyond 6 weeks from the prior ipilimumab dose, then subsequent ipilimumab doses should rescheduled to maintain the 6 week interval between consecutive ipilimumab doses. In Arm C, dose delay of ipilimumab which results in no ipilimumab dosing for >12 weeks requires ipilimumab discontinuation.

### Dose Delay Criteria for Arm D (Platinum Doublet Chemotherapy)

In Arm D, dosing of both drugs in the platinum doublet chemotherapy regimen selected should be delayed for any of the following on the Day 1 of each cycle: Absolute neutrophil count (ANC)<1,500/mm³, Platelets <100,000/mm³; Any Grade greater than or equal to 2 non-skin, non-hematologic, drug-related adverse event (excluding Grade 2 alopecia, Grade 2 fatigue, and Grade 2 laboratory abnormalities); Any Grade greater than or equal to 3 skin, drug-related adverse event; Any Grade greater than or equal to 3 drug-related laboratory abnormality, with the following exceptions for lymphopenia, AST, ALT, or total bilirubin: Grade 3 lymphopenia does not require dose delay. If a subject has a baseline AST, ALT or total bilirubin that is within normal limits, delay dosing for drug-related Grade greater than or equal to 2 toxicity; If a subject has baseline AST, ALT, or total bilirubin within the Grade 1 toxicity range, delay dosing for drug-related Grade greater than or equal to 3 toxicity; Any adverse event, laboratory abnormality, or intercurrent illness which, in the judgment of the investigator, warrants delaying the dose of study medication.

In addition, subjects receiving cisplatin with pemetrexed or gemcitabine must discontinue cisplatin if the calculated creatinine clearance decreases to <50 mL/min (based on the Cockroft Gault formula). The other drug (pemetrexed or gemcitabine) may be continued, and the platinum agent may, at the investigator's discretion, be switched to carboplatin for the remainder of the platinum doublet cycles (up to 6 cycles in total) when the subject meets retreatment criteria.

Subjects receiving gemcitabine with cisplatin or carboplatin should omit the Day 8 gemcitabine dose for any of the following on Day 8 of any cycle: ANC <1,000/mm³; Platelets <75,000/mm³.

If any non-hematologic adverse event meeting the dose delay criteria above is felt to be related to only one particular agent in the platinum doublet chemotherapy regimen, then that agent alone may be omitted for that cycle while the other agent is given. In order to maintain synchronized dosing of the regimen, the omitted agent should be resumed with the next scheduled cycle once the AE has improved and retreatment criteria are met.

If both drugs in the platinum doublet chemotherapy regimen are delayed, then the subject should be re-evaluated weekly or more frequently if clinically indicated until re-treatment criteria are met.

### Dose Reductions

There will be no dose reductions for nivolumab or ipilimumab.

Dose reductions of platinum doublet chemotherapy may be required. Chemotherapy dose reductions are permanent; once the dose of any chemotherapy agent is reduced, it may not be re-escalated in subsequent cycles, except as noted when starting pemetrexed maintenance therapy. The dose reductions for each agent in the platinum doublet chemotherapy regimen are not linked and may be adjusted independently as summarized below.

Possible dose modifications for chemotherapeutic agents are shown in Table 9.

TABLE 9
Dose Modifications of Chemotherapeutic Agents

| Dose Level | Gemcitabine | Pemetrexed | Cisplatin | Carboplatin |
|---|---|---|---|---|
| First dose | 950 mg/m² | 375 mg/m² | 56 mg/m² | AUC 5 with |
| reduction | (with cisplatin) or | | | pemetrexed or |
| | 750 mg/m² | | | AUC 4 with |
| | (with carboplatin) | | | gemcitabine |
| Second dose | 625 mg/m² | 250 mg/m² | 38 mg/m² | AUC 4 with |
| reduction | (with cisplatin) or | | | pemetrexed or |
| | 500 mg/m² | | | AUC 3 with |
| | (with carboplatin) | | | gemcitabine |
| Third dose | Discontinue | Discontinue | Discontinue | Discontinue |
| reduction | | | | |

Any subjects with two prior dose reductions for one agent who experiences a toxicity that would cause a third dose reduction must be discontinued from that agent.

Dose modifications for hematologic toxicities (according to CTCAE version 4) are summarized in Table 10. Dose adjustments are based on nadir blood counts (assessed as per local standards) since the preceding drug administration. Dose level adjustments for platinum doublet chemotherapy are relative to that of the preceding administration. Generally, both chemotherapy agents in the platinum doublet chemotherapy regimen should be dose reduced together for hematologic toxicity. After the first cycle, growth factors may be used to assist hematologic recovery. Use local standards of care in the use of these supportive measures. Additionally, prophylactic antibiotics may be used according to local standards of care. Please report any antibiotic or growth factor use on the eCRF.

TABLE 10
Dose Modifications for Hematologic Toxicity (Based on Nadir Counts)

| | | | | |
|---|---|---|---|---|
| Toxicity | Gemcitabine | Pemetrexed | Cisplatin | Carboplatin |

Neutrophil Count Decreased

| | | | | |
|---|---|---|---|---|
| Grade 4 | Reduce one | Reduce one | Reduce one | Reduce one |
| (<500/ | dose level | dose level | dose level | dose level |
| mm³ or <0.5 × | | | | |
| 10⁹/L) | | | | |

Platelet Count Decreased

| | | | | |
|---|---|---|---|---|
| Grade 3 | Reduce one | Reduce one | Reduce one | Reduce one |
| (25,000-<50,000/ | dose level | dose level | dose level | dose level |
| mm³; 25.0-<50.0 × | | | | |
| 10⁹/L) | | | | |
| Grade 4 | Reduce one | Reduce one | Reduce one | Reduce one |
| (<25,000/ | dose level | dose level | dose level | dose level |
| mm³; <25.0 × | | | | |
| 10⁹/L) | | | | |

Dose adjustments for platinum doublet chemotherapy for non-hematologic toxicities during treatment are described in Table 11. All dose reductions should be made based on the worst grade toxicity. Subjects experiencing any of the toxicities detailed in Table 10 during the previous cycle should have their chemotherapy delayed until retreatment criteria are met and then reduced for all subsequent cycles by 1 dose level or discontinued as appropriate. Dose levels for the two drugs in the platinum-doublet chemotherapy regimen are not linked and may be reduced independently, as summarized in the table below.

TABLE 11
Dose Modifications for Non-hematologic Toxicity

| Toxicity | Gemcitabine | Pemetrexed | Cisplatin | Carboplatin |
|---|---|---|---|---|
| Febrile Neutropenia | Reduce one | Reduce one | Reduce one | Reduce one |
| Grade ≥3 | dose level | dose level | dose level | dose level |
| Diarrhea | Reduce one | Reduce one | No change | No change |
| Grade ≥3 | dose level | dose level | | |
| Allergic reaction^{a} | Discontinue | Discontinue | Discontinue | Discontinue |
| Grade ≥3 | | | | |
| Neuropathy | No change | No change | Reduce one | No change |
| Grade 2 | | | dose level | |
| Neuropathy | Discontinue | Discontinue | Discontinue | Discontinue |
| Grade ≥3 | | | | |
| Calculated creatinine | No change | No change | Discontinue | No change |
| clearance <50 mL/min | | | | |
| Other Grade ≥3 | Adjust as | Adjust as | Adjust as | Adjust as |
| toxicity (except for | medically | medically | medically | medically |
| fatigue and transient | indicated | indicated | indicated | indicated |
| arthralgia and myalgia) | | | | |

^{a}Only the drug(s) causing the hypersensitivity reaction or acute infusion reaction (≥Grade 3) require(s)discontinuation. All other drugs may be continued.

### Criteria to Resume Nivolumab Dosing

Subjects may resume treatment with nivolumab when the drug-related AE(s) resolve(s) to Grade less than or equal to 1 or baseline, with the following exceptions: Subjects may resume treatment in the presence of Grade 2 fatigue; Subjects who have not experienced a Grade 3 drug-related skin AE may resume treatment in the presence of Grade 2 skin toxicity; Subjects with baseline Grade 1 AST/ALT or total bilirubin who require dose delays for reasons other than a 2-grade shift in AST/ALT or total bilirubin may resume treatment in the presence of Grade 2 AST/ALT OR total bilirubin. Subjects with combined Grade 2 AST/ALT and total bilirubin values meeting discontinuation parameters should have treatment permanently discontinued.

Drug-related pulmonary toxicity, diarrhea, or colitis must have resolved to baseline before treatment is resumed. Subjects with persistent Grade 1 pneumonitis after completion of a steroid taper over at least 1 month may be eligible for retreatment if discussed with and approved by the BMS Medical Monitor.

Drug-related endocrinopathies adequately controlled with only physiologic hormone replacement may resume treatment after consultation with the BMS Medical Monitor.

Subjects who delay study treatment due to any Grade greater than or equal to 3 amylase or lipase abnormality that is not associated with symptoms or clinical manifestations of pancreatitis, and that is assessed by the investigator to be related to ipilimumab and not to nivolumab, may resume nivolumab when the amylase or lipase abnormality has resolved to Grade <3. Dose delay of nivolumab which results in treatment interruption of >6 weeks requires treatment discontinuation.

### Criteria to Resume Ipilimumab Dosing

Subjects may resume treatment with nivolumab and ipilimumab when drug-related AE(s) resolve(s) to Grade 1 or baseline value, with the following exceptions: Subjects may resume treatment in the presence of Grade 2 fatigue; Subjects who have not experienced a Grade 3 drug-related skin AE may resume treatment in the presence of Grade 2 skin toxicity; Subjects with baseline Grade 1 AST/ALT or total bilirubin who require dose delays for reasons other than a 2-grade shift in AST/ALT or total bilirubin may resume treatment in the presence of Grade 2 AST/ALT or total bilirubin. Subjects with combined Grade 2 AST/ALT and total bilirubin values meeting discontinuation parameters should have treatment permanently discontinued.

Drug-related pulmonary toxicity, diarrhea, or colitis must have resolved to baseline before treatment is resumed.

Subjects who received systemic corticosteroids for management of any drug-related toxicity must be off corticosteroids or have tapered down to an equivalent dose of prednisone ±10 mg/day.

Drug-related endocrinopathies adequately controlled with only physiologic hormone replacement may resume treatment after consultation with a Medical Monitor.

Dose delay of ipilimumab which results in no ipilimumab dosing for >12 weeks requires ipilimumab discontinuation.

In Arm C, ipilimumab may not be resumed sooner than 6 weeks (+/−5 days) after the prior ipilimumab dose.

In Arm C, in general, subjects who meet criteria to resume ipilimumab will also have met criteria to resume nivolumab, so it should be feasible to synchronize dosing of both drugs when resuming ipilimumab. In order to facilitate this, the dosing days of nivolumab and ipilimumab may be adjusted within the permitted +/−5 day window, as long as consecutive nivolumab doses are given at least 12 days apart.

One exception to note is when ipilimumab and nivolumab doses are delayed due to drug-related Grade greater than or equal to 3 amylase or lipase abnormalities not associated with symptoms or clinical manifestations of pancreatitis. If the investigator assesses the Grade greater than or equal to 3 amylase or lipase abnormality to be related to ipilimumab and not related to nivolumab, nivolumab may be resumed when the amylase or lipase abnormality resolves to Grade <3 but ipilimumab may only be resumed when the amylase or lipase abnormality resolves to Grade 1 or baseline.

Criteria to Resume Treatment with Platinum Doublet Chemotherapy

Subjects may resume treatment with platinum doublet chemotherapy when the ANC returns to greater than or equal to 1,500/mm³, the platelet count returns to greater than or equal to 100,000/mm³, and all other drug-related toxicities have returned to baseline or Grade less than or equal to 1 (or Grade less than or equal to 2 for alopecia and fatigue).

If a subject fails to meet criteria for re-treatment, then re-treatment should be delayed, and the subject should be re-evaluated weekly or more frequently as clinically indicated. Any subject who fails to recover from toxicity attributable to platinum doublet chemotherapy to baseline or Grade less than or equal to 1 (except Grade 2 alopecia and fatigue) within 6 weeks from the last dose given should discontinue the drug(s) that caused the delay.

### Nivolumab Dose Discontinuation

Treatment with nivolumab should be permanently discontinued for any of the following: Any Grade 2 drug-related uveitis or eye pain or blurred vision that does not respond to topical therapy and does not improve to Grade 1 severity within the re-treatment period OR requires systemic treatment; Any Grade greater than or equal to 2 drug-related pneumonitis or interstitial lung disease that does not resolve to dose delay and systemic steroids (also see Pulmonary Adverse Event Management Algorithm); Any Grade 3 drug-related bronchospasm, hypersensitivity reaction, or infusion reaction, regardless of duration; Any Grade 3 non-skin, drug-related adverse event lasting >7 days, with the following exceptions for uveitis, pneumonitis, bronchospasm, diarrhea, colitis, neurologic toxicity, hypersensitivity reactions, infusion reactions, endocrinopathies, and laboratory abnormalities; Grade 3 drug-related uveitis, pneumonitis, bronchospasm, diarrhea, colitis, neurologic toxicity, hypersensitivity reaction, or infusion reaction of any duration requires discontinuation; Grade 3 drug-related endocrinopathies adequately controlled with only physiologic hormone replacement do not require discontinuation; Grade 3 drug-related laboratory abnormalities do not require treatment discontinuation except:

Grade 3 drug-related thrombocytopenia >7 days or associated with bleeding requires discontinuation; Any drug-related liver function test (LFT) abnormality that meets the following criteria require discontinuation (also see Hepatic Adverse Event Management Algorithm): AST or ALT >5-10×ULN for >2 weeks, AST or ALT >10×ULN, Total bilirubin >5×ULN, Concurrent AST or ALT >3×ULN and total bilirubin >2×ULN.

Any Grade 4 drug-related adverse event or laboratory abnormality, except for the following events, which do not require discontinuation: Grade 4 neutropenia less than or equal to 7 days; Grade 4 lymphopenia or leukopenia; Isolated Grade 4 amylase or lipase abnormalities that are not associated with symptoms or clinical manifestations of pancreatitis and decrease to <Grade 4 within 1 week of onset; Isolated Grade 4 electrolyte imbalances/abnormalities that are not associated with clinical sequelae and are corrected with supplementation/appropriate management within 72 hours of their onset; Grade 4 drug-related endocrinopathy adverse events such as adrenal insufficiency, ACTH deficiency, hyper- or hypothyroidism, or glucose intolerance, which resolve or are adequately controlled with physiologic hormone replacement (corticosteroids, thyroid hormones) or glucose controlling agents, respectively, may not require discontinuation after discussion with and approval from the Medical Monitor.

Dosing delays lasting >6 weeks from the previous dose that occur for non-drug-related reasons may be allowed if approved by the BMS medical monitor. Prior to re-initiating treatment in a subject with a dosing delay lasting >6 weeks, the medical monitor must be consulted. Tumor assessments should continue as per protocol even if dosing is delayed. Periodic study visits to assess safety and laboratory studies should also continue every 6 weeks or more frequently if clinically indicated during such dosing delays.

Any adverse event, laboratory abnormality, or intercurrent illness which, in the judgment of the Investigator, presents a substantial clinical risk to the subject with continued nivolumab dosing.

The assessment for discontinuation of nivolumab should be made separately from the assessment made for discontinuation of ipilimumab. Although there is overlap among the discontinuation criteria, if discontinuation criteria are met for ipilimumab but not for nivolumab, treatment with nivolumab may continue if ipilimumab is discontinued.

If a subject in Arm B meets the criteria for discontinuation of ipilimumab but not nivolumab prior to completion of the first 4 cycles, treatment with nivolumab may not resume until the adverse event has fully resolved, and the subject has discontinued steroids, if they were required for treatment of the adverse event.

If a subject in Arm B (during the first 4 cycles) or Arm C meets criteria for discontinuation and investigator is unable to determine whether the event is related to both or one study drug, the subject should discontinue both nivolumab and ipilimumab and be taken off the treatment phase of the study.

### Ipilimumab Dose Discontinuation

Ipilimumab should be permanently discontinued if any of the following criteria are met: Any grade greater than or equal to Grade 2 drug-related uveitis or eye pain or blurred vision that does not respond to topical therapy and does not improve to Grade 1 severity within 2 weeks OR requires systemic treatment; Any Grade greater than or equal to 3 bronchospasm or other hypersensitivity reaction; Any other Grade 3 non-skin, drug-related adverse with the following exceptions for laboratory abnormalities, grade 3 nausea and vomiting, grade 3 neutropenia and thrombocytopenia, and symptomatic endocrinopathies which resolved (with or without hormone substitution); Any drug-related liver function test (LFT) abnormality that meets the following criteria require discontinuation: AST or ALT >8×ULN, Total bilirubin >5×ULN, or Concurrent AST or ALT >3×ULN and total bilirubin >2×ULN; Any Grade 4 drug-related adverse event or laboratory abnormality, except for the following events, which do not require discontinuation: Grade 4 neutropenia less than or equal to 7 days, Grade 4 lymphopenia or leukopenia; Isolated Grade 4 amylase or lipase abnormalities which are not associated with symptoms or clinical manifestations of pancreatitis; Isolated Grade 4 electrolyte imbalances/abnormalities that are not associated with clinical sequelae and are corrected with supplementation/appropriate management within 72 hours of their onset; Grade 4 drug-related endocrinopathy adverse events such as adrenal insufficiency, ACTH deficiency, hyper- or hypothyroidism, or glucose intolerance, which resolve or are adequately controlled with physiologic hormone replacement (corticosteroids, thyroid hormones) or glucose controlling agents, respectively, may not require discontinuation after discussion with and approval from the Medical Monitor.

Any treatment delay resulting in no ipilimumab dosing for >12 weeks in Arm C with the following exceptions: Dosing delays to manage drug-related adverse events, such as prolonged steroid tapers, are allowed. Prior to re-initiating treatment in a subject with a dosing delay lasting >12 weeks in Arm C, the medical monitor must be consulted. Tumor assessments should continue as per protocol even if dosing is delayed.

Dosing delays resulting in no ipilimumab dosing for >12 weeks in Arm C that occur for non-drug-related reasons may be allowed if approved by the medical monitor. Prior to re-initiating treatment in a subject with a dosing delay lasting >12 weeks in Arm C, the medical monitor must be consulted. Tumor assessments should continue as per protocol even if dosing is delayed.

The assessment for discontinuation of ipilimumab should be made separately from the assessment made for discontinuation of nivolumab. Although there is overlap among the discontinuation criteria, if discontinuation criteria are met for ipilimumab but not for nivolumab, treatment with nivolumab may continue if ipilimumab is discontinued.

If a subject in Arm B meets the criteria for discontinuation of ipilimumab but not nivolumab, treatment with nivolumab may not resume until the adverse event has fully resolved and the subject has discontinued steroids, if they were required for treatment of the adverse event. The relationship to ipilimumab should be well documented in the source documents.

If a subject in Arm B, during the first 4 cycles, or Arm C meets criteria for discontinuation and investigator is unable to determine whether the event is related to both or one study drug, the subject should discontinue both nivolumab and ipilimumab and be taken off the treatment phase of the study.

### Platinum Doublet Chemotherapy Dose Discontinuation

Except where specified below, both chemotherapy drugs in the platinum doublet chemotherapy regimen should be discontinued for any of the following: Any Grade greater than or equal to 3 peripheral neuropathy; Grade greater than or equal to 3 drug-related thrombocytopenia associated with clinically significant bleeding; Any drug-related liver function test (LFT) abnormality that meets the following criteria requires discontinuation: AST or ALT >5-10×ULN for >2 weeks, AST or ALT >10×ULN, Total bilirubin >5×ULN, or Concurrent AST or ALT >3×ULN and total bilirubin >2×ULN; Any cisplatin-related decrease in creatinine clearance to <50 mL/min (using the Cockroft Gault formula) requires discontinuation of cisplatin; Any drug-related adverse event which recurs after two prior dose reductions for the same drug-related adverse event requires discontinuation of the drug(s) which was/were previously dose reduced; Any Grade greater than or equal to 3 drug-related hypersensitivity reaction or infusion reaction requires discontinuation of the drug(s) felt to be causing the reaction. The drug not felt to be related to the hypersensitivity reaction or infusion reaction may be continued; Any Grade 4 drug-related adverse event which the investigator deems is inappropriate to be managed by dose reduction(s) requires discontinuation of the drug(s) felt to be causing the event. The drug not felt to be related to the event may be continued; Any event that leads to delay in dosing of any study drug(s) for >6 weeks from the previous dose requires discontinuation of that drug(s) with the following exception:

Dosing delays lasting >6 weeks from the previous dose that occur for non-drug-related reasons may be allowed if approved by the BMS medical monitor. Prior to re-initiating treatment in a subject with a dosing delay lasting >6 weeks, the BMS medical monitor must be consulted. Periodic study visits to assess safety and laboratory studies should also continue every 6 weeks or more frequently if clinically indicated during such dosing delays.

Any adverse event, laboratory abnormality, or intercurrent illness which, in the judgment of the Investigator, presents a substantial clinical risk to the subject with continued platinum doublet chemotherapy dosing. Investigators should consult local labeling for the chemotherapy drugs being administered to any given subject for additional guidance on dose discontinuation.

Note that subjects receiving gemcitabine/cisplatin who discontinue cisplatin alone may, at the investigator's discretion, be switched to gemcitabine/carboplatin for the remainder of the platinum doublet cycles (up to 6 cycles in total). Subjects receiving pemetrexed/cisplatin who discontinue cisplatin alone may, at the investigator's discretion, be switched to pemetrexed/carboplatin for the remainder of the platinum doublet cycles (up to 6 cycles in total).

### Treatment Beyond Disease Progression (Arms A, B, and C)

Some subjects treated with immunotherapy may derive clinical benefit despite initial evidence of PD. Subjects will be permitted to continue on nivolumab in Arm A or nivolumab+ipilimumab in Arms B and C for treatment beyond initial RECIST 1.1 defined PD as long as they meet the following criteria: Investigator-assessed clinical benefit and no rapid disease progression; Subject is tolerating study treatment; Stable performance status; Treatment beyond progression will not delay an imminent intervention to prevent serious complications of disease progression (e.g., CNS metastases); and Subject provides written informed consent prior to receiving additional nivolumab and or ipilimumab treatment, using an ICF describing any reasonably foreseeable risks or discomforts, or other alternative treatment options.

A follow-up scan should be performed within six (6) weeks ±5 days of original PD to determine whether there has been a decrease in the tumor size, or continued progression of disease. Subsequent scans should be performed every twelve (12) weeks until further progression is determined.

If the investigator feels that the subject continues to achieve clinical benefit by continuing treatment, the subject should remain on the trial and continue to receive monitoring according to the Time and Events Schedule on Tables 18-20 (Example 1).

For the subjects in Arms A, B, and C who continue study therapy beyond progression, further progression is defined as an additional 10% increase in tumor burden from time of initial PD. This includes an increase in the sum of diameters of all target lesions and/or the diameters of new measurable lesions compared to the time of initial PD. Nivolumab and/or ipilimumab treatment should be discontinued permanently upon documentation of further progression.

New lesions are considered measureable at the time of initial progression if the longest diameter is at least 10 mm (except for pathological lymph nodes which must have a short axis of at least 15 mm). Any new lesion considered non-measureable at the time of initial progression may become measureable and therefore included in the tumor burden if the longest diameter increases to at least 10 mm (except for pathological lymph nodes which must have a short axis of at least 15 mm). In situations where the relative increase in total tumor burden by 10% is solely due to inclusion of new lesions which become measurable, these new lesions must demonstrate an absolute increase of at least 5 mm.

### Management Algorithms for Immuno-Oncology Agents

Immuno-oncology (I-O) agents are associated with AEs that can differ in severity and duration than AEs caused by other therapeutic classes. Nivolumab and ipilimumab are considered immuno-oncology agents in this protocol. Early recognition and management of AEs associated with immuno-oncology agents may mitigate severe toxicity. Management Algorithms have been developed to assist investigators in assessing and managing the following groups of AEs: Gastrointestinal; Renal; Pulmonary; Hepatic; Endocrinopathy; Skin; and Neurological. The algorithms are found in both the nivolumab and ipilimumab Investigator Brochures.

### Treatment of Nivolumab or Ipilimumab Infusion Reactions

Since nivolumab and ipilimumab contain only human immunoglobulin protein sequences, they are unlikely to be immunogenic and induce infusion or hypersensitivity reactions. However, if such a reaction were to occur, it might manifest with fever, chills, rigors, headache, rash, pruritus, arthralgias, hypo- or hypertension, bronchospasm, or other symptoms. Infusion reactions should be graded according to NCI CTCAE (Version 4.0) guidelines.

Treatment recommendations are provided below and may be modified based on local treatment standards and guidelines, as appropriate:

For Grade 1 symptoms: (mild reaction; infusion interruption not indicated; intervention not indicated), remain at bedside and monitor subject until recovery from symptoms. The following prophylactic pre-medications are recommended for future infusions: diphenhydramine 50 mg (or equivalent) and/or acetaminophen/paracetamol 325 to 1000 mg at least 30 minutes before additional nivolumab or ipilimumab administrations.

For Grade 2 symptoms: (moderate reaction requires therapy or infusion interruption but responds promptly to symptomatic treatment [e.g., antihistamines, non-steroidal anti-inflammatory drugs, narcotics, corticosteroids, bronchodilators, IV fluids]; prophylactic medications indicated for less than or equal to 24 hours), stop the nivolumab or ipilimumab infusion, begin an IV infusion of normal saline, and treat the subject with diphenhydramine 50 mg IV (or equivalent) and/or acetaminophen/paracetamol 325 to 1000 mg; remain at bedside and monitor subject until resolution of symptoms. Corticosteroid and/or bronchodilator therapy may also be administered as appropriate. If the infusion is interrupted, then restart the infusion at 50% of the original infusion rate when symptoms resolve; if no further complications ensue after 30 minutes, the rate may be increased to 100% of the original infusion rate. Monitor subject closely. If symptoms recur, then no further nivolumab or ipilimumab will be administered at that visit. Administer diphenhydramine 50 mg IV, and remain at bedside and monitor the subject until resolution of symptoms.

For future infusions, the following prophylactic pre-medications are recommended: diphenhydramine 50 mg (or equivalent) and/or acetaminophen/paracetamol 325 to 1000 mg should be administered at least 30 minutes before nivolumab or ipilimumab infusions. If necessary, corticosteroids (up to 25 mg of SoluCortef or equivalent) may be used.

For Grade 3 or 4 symptoms: (severe reaction, Grade 3: prolonged [i.e., not rapidly responsive to symptomatic medication and/or brief interruption of infusion]; recurrence of symptoms following initial improvement; hospitalization indicated for other clinical sequelae [e.g., renal impairment, pulmonary infiltrates]. Grade 4: Life threatening; pressor or ventilatory support indicated), immediately discontinue infusion of nivolumab or ipilimumab. Begin an IV infusion of normal saline and treat the subject as follows: Recommend bronchodilators, epinephrine 0.2 to 1 mg of a 1:1000 solution for subcutaneous administration or 0.1 to 0.25 mg of a 1:10,000 solution injected slowly for IV administration, and/or diphenhydramine 50 mg IV with methylprednisolone 100 mg IV (or equivalent), as needed. Subject should be monitored until the investigator is comfortable that the symptoms will not recur. Nivolumab or ipilimumab will be permanently discontinued.

In case of late-occurring hypersensitivity symptoms (e.g., appearance of a localized or generalized pruritus within 1 week after treatment), symptomatic treatment may be given (e.g., oral antihistamine or corticosteroids).

Follow-up and survival procedures are shown in Table 12.

TABLE 12
Follow-up and Survival Procedures (CA209227) - All subjects

| | | | |
|---|---|---|---|
| | Follow-Up^{a} | Survival | |
| Procedure | Visits 1 & 2 | Follow-up Visits^{b} | Notes |

SAFETY ASSESSMENTS

| | | | |
|---|---|---|---|
| Targeted Physical Examination | X | | To assess for potential late emergent study drug related issues. |
| Vital Signs | X | | |
| Adverse Event Assessment | X | X | |
| Review of Concomitant | X | | |
| Medications | | | |
| Laboratory Tests | X | | Required at Visit 1. Repeat at Visit 2 only if study drug related toxicity |
| | | | persists. |

EFFICACY ASSESSMENTS

| | | | |
|---|---|---|---|
| Radiographic Tumor Assessment | X | X | For subjects who discontinue study treatment for reasons other than PD, |
| (CT/MRI of chest, abdomen, | | | follow up scans should be performed every 6 weeks (±1 wk) up to first 12 |
| pelvis and known sites of disease) | | | months (week 48), then every 12 weeks until PD, lost to follow-up, or |
| | | | withdrawal of consent |
| | | | *Radiographic assessments for subjects who have not experienced PD must |
| | | | be obtained every 6 weeks (±7 days), and not delayed until follow-up visits 1 |
| | | | & 2. |
| Patient Reported Outcomes | X | EQ-5D only | Both the LCSS and EQ-5D will be given in FU Visits 1 & 2. In Survival |
| Assessment (PRO) | | | Visits, EQ-5D is collected every 3 months for the first year of the Follow-Up |
| | | | Phase, then every 6 months thereafter. |
| Pharmacokinetic & | X | | (For subjects treated in arms A, B, and C) |
| Immunogenicity | | | |
| Collection of Survival Status and | X | X | Collect every 3 months in Survival Visits until death, lost to follow-up, or |
| Subsequent Therapy Information | | | withdrawal of study consent. May be performed by phone contact or office |
| | | | visit. |

^{a}Follow-Up Visit 1 to occur 35 days from the last dose (±7 days) or coinciding with the date of discontinuation of study drug (±7 days) if the date of discontinuation is greater than 42 days from the last dose. Follow-Up Visit 2 to occur 80 days from Follow-Up Visit 1 (±7 days).
^{b}Survival Follow-Up Visits to occur approximately every 3 months from Follow-Up Visit 2.

### Safety Assessments

At baseline, a medical history will be obtained to capture relevant underlying conditions. The baseline examinations should include weight, height, ECOG Performance Status, blood pressure (BP), heart rate (HR), temperature, and oxygen saturation by pulse oximetry at rest (also monitor amount of supplemental oxygen if applicable) should be performed within 28 days prior to first dose. Baseline signs and symptoms are those that are assessed within 14 days prior to first dose. Concomitant medications will be collected from within 14 days prior to the first dose through the study treatment period.

Baseline local laboratory assessments should be done within 14 days prior to first dose and are to include: CBC w/differential, LFTs (ALT, AST, total bilirubin, alkaline phosphatase), BUN or serum urea level, creatinine, albumin, Ca, Mg, Na, K, Cl, phosphate, LDH, glucose, amylase, lipase, Thyroid function tests includes TSH, free T4, and free T3.

The following baseline local laboratory assessments should be done within 28 days prior to randomization: Hepatitis B and C testing (HBV sAg and HCV antibody or HCV RNA).

Pregnancy testing for WOCBP (done locally) must be performed within 24 hours prior to the Day 1 at baseline and then every 4 weeks (2 cycles) ±3 days for subjects assigned to arms A and C and every 3 weeks (each cycle) ±3 days for subjects (for subjects assigned to arms B and D). Pregnancy testing must be within 24 hours prior to Day 1 of each treatment cycle (prior to dosing).

While on-study the following local laboratory assessments are to be done within 3 days prior to each dose: CBC with differential, LFTs (ALT, AST, total bilirubin, alkaline phosphatase), BUN or serum urea level, creatinine, albumin, Ca, Mg, Na, K, Cl, phosphate, LDH, glucose, amylase, and lipase. Thyroid function testing is to be done every 6 weeks.

Subjects will be evaluated for safety if they have received any study drug. Toxicity assessments will be continuous during the treatment phase. During the safety follow-up phase toxicity assessments should be done in person.

Adverse events and laboratory values will be graded according to the NCI-CTCAE version 4.0. On-study weight, ECOG performance status, and vital signs should be assessed at each on-study visit prior to dosing. Vital signs should also be taken as per institutional standard of care prior to, during and after infusions. Oxygen saturation by pulse oximetry at rest and on exertion (also monitor amount of supplemental oxygen if applicable) should be assessed at each on-study visit prior to dosing.

On treatment local laboratory assessments are to be completed within 72 hours prior to dosing. Additional measures, including non-study required laboratory tests, should be performed as clinically indicated or to comply with local regulations. Laboratory toxicities (e.g., suspected drug induced liver enzyme evaluations) will be monitored during the follow-up phase via on site/local labs until all study drug related toxicities resolve, return to baseline, or are deemed irreversible.

Oxygen saturation by pulse oximetry should be obtained prior to each dosing and at any time a subject has any new or worsening respiratory symptoms. A reading at rest and on exertion should be obtained at each time point. The extent of the exertion should be based on the judgment of the investigator, but should remain consistent for each individual subject throughout the study. If the patient's subject's status changes, the investigator can alter the extent of exertion based on their medical judgment. If a subject shows changes on pulse oximetry or other pulmonary related signs (hypoxia, fever) or symptoms (e.g., dyspnea, cough, fever) consistent with possible pulmonary adverse events, the patient subject should be immediately evaluated to rule out pulmonary toxicity. An algorithm for the management of suspected pulmonary toxicity can be found in the nivolumab Investigator's Brochure.

Some of the assessments referred to in this section may not be captured as data in the eCRF. They are intended to be used as safety monitoring by the treating physician.

Eastern Cooperative Oncology Group (ECOG) Performance Status will be evaluated and documented at Screening and within 72 hours prior to each dosing visit (See Table 7).

WOCBP are required to have pregnancy tests performed. WOCBP must exhibit a negative serum or urine pregnancy (minimum sensitivity 25 IU/L or equivalent units of HCG within 24 hours prior to Day 1 of each treatment cycle

Thyroid function testing will be performed. At Screening, thyroid function testing is to include TSH, free T3 and free T4. At subsequent time points, thyroid function testing consists of TSH only. However, if the TSH is abnormal, reflexive testing of free T3 and free T4 are to be performed.

Management algorithms for suspected endocrinopathy adverse events (including abnormal thyroid function) can be found in the nivolumab investigator brochure.

All subjects who have met the eligibility criteria are required to have a 12-lead ECG performed during Screening. If clinically indicated, additional ECGs may be obtained during the study.

Contrast enhanced CT with PO/IV contrast or contrast enhanced MM are imaging modalities for assessing radiographic tumor response. If a subject has a known allergy to contrast material, please use local prophylaxis standards to obtain the assessment with contrast if at all possible, or use the alternate modality. In cases where contrast is strictly contraindicated, a non-contrast scan will suffice. Should a subject have a contraindication for CT IV contrast, a non-contrast CT of the chest and a contrast enhanced MRI of the abdomen and pelvis may be obtained.

Use of CT component of a PET/CT scanner: Combined modality scanning such as with FDG-PET/CT is increasingly used in clinical care, and is a modality/technology that is in rapid evolution; therefore, the recommendations outlined here may change rather quickly with time. At present, low dose or attenuation correction CT portions of a combined FDG-PET/CT are of limited use in anatomically based efficacy assessments and it is therefore suggested that they should not be substituted for dedicated diagnostic contrast enhanced CT scans for anatomically based RECIST measurements. However, if a site can document that the CT performed as part of a FDG-PET/CT is of identical diagnostic quality to a diagnostic CT (with IV and oral contrast) then the CT portion of the FDG-PET/CT can be used for RECIST 1.1 measurements.

MRI of brain is required at screening in order to rule out active metastatic disease.

Bone scan or PET scan is not adequate for assessment of RECIST 1.1 response in target lesions. In selected circumstances where such modalities are the sole modality used to assess certain non-target organs, those non-target organs may be evaluated less frequently. For example, bone scans may need to be repeated only when complete response is identified in target disease or when progression in bone is suspected.

Screening assessments are to be performed within 28 days prior to randomization. In addition to the chest, abdomen, pelvis, and brain (to rule out brain metastases), all known sites of disease should be assessed at baseline. Subsequent assessments should include chest, abdomen, pelvis, and all known sites of disease using the same imaging method and technique as was used at baseline.

Radiographic tumor response will be assessed at Week 6 (±7 days) from randomization date, then every 6 weeks (±7 days) for the first 12 months (until week 48) and every 12 weeks (±7 days) thereafter, until disease progression is documented or treatment is discontinued (whichever occurs later). Subjects with a history of brain metastasis may have surveillance MM approximately every 12 weeks from the date of first dose, or sooner if clinically indicated.

In addition, subjects receiving nivolumab and/or ipilimumab treatment beyond progression must continue tumor assessments until such treatment has been discontinued.

A primary endpoint is overall survival (OS) in all randomized subjects. Secondary efficacy endpoints of the study include PFS and ORR, based on BICR assessment, in all randomized subjects. All randomized subjects will be monitored by radiographic assessment on an every-6-week schedule every 6 weeks (±7 days) for the first 12 months (until week 48) and every 12 weeks (±7 days) thereafter [beginning from the first on-study assessment on week 6 (±7 days)], to determine changes in tumor size. RECIST 1.1 criteria will be used for the assessment.

### Pharmacokinetic and Immunogenicity Assessments

Samples for PK and immunogenicity assessments will be collected for all subjects receiving nivolumab and ipilimumab as described in Table 13 to Table 15. All time points are relative to the start of study drug administration. All on-treatment time points are intended to align with days on which study drug is administered, if dosing occurs on a different day, the PK and immunogenicity sampling should be adjusted accordingly.

TABLE 13
Pharmacokinetic (PK) and Immunogenicity Sample Collections- Nivolumab Arm A

| | Event | Time | Pharmacokinetic | Immunogenicity |
|---|---|---|---|---|
| Study Day^{a} | (Relative | (Relative | Blood | Blood |
| (1 Cycle = 2 | To Dosing) | To Dosing) | Sample for | Sample for |
| Weeks) | Hour | Hour:Min | Nivolumab | Nivolumab |
| C1D1 | Predose^{a} | 00:00 | X | X |
| C3D1 | Predose^{b} | 00:00 | X | X |
| C8D1 | Predose^{b} | 00:00 | X | X |
| D1 of every 8th | Predose^{b} | 00:00 | X | X |
| cycle after | | | | |
| C8D1 until end | | | | |
| of study | | | | |
| First 2 Follow-up visits | | | X | X |
| (approximately up to | | | | |
| 100 days from the | | | | |
| discontinuation | | | | |
| of study drug | | | | |

^{a}If a subject permanently discontinues study drug treatment during the sampling period, they will move to sampling at follow-up visits
^{b}All pre-dose samples for nivolumab should be taken prior to the start of nivolumab infusion

TABLE 14
Pharmacokinetic (PK) and Immunogenicity Sample Collections- Nivolumab + Ipilimumab Arm B

| | Study Day^{b} | | | | | | |
|---|---|---|---|---|---|---|---|
| | 1 Cycle = 3 | Event | Time | Pharmacokinetic | Immunogenicity | Pharmacokinetic | Immunogenicity |
| | Weeks (Part A) | (Relative | (Relative | Blood | Blood | Blood | Blood |
| | 1 Cycle = 2 | To Dosing) | To Dosing) | Sample for | Sample for | Sample for | Sample for |
| Part^{a} | Weeks (Part B) | Hour | Hour:Min | Nivolumab | Nivolumab | Ipilimumab | Ipilimumab |
| A | C1D1 | Predose^{c} | 00:00 | X | X | X | X |
| A | C3D1 | Predose^{c} | 00:00 | X | X | X | X |
| B | C5D1 | Predose^{c} | 00:00 | X | X | X | X |
| B | C13D1 | Predose^{c} | 00:00 | X | X | X | X |
| B | D1 of every 8th | Predose^{c} | 00:00 | X | X | X | X |
| | Cycle after | | | | | | |
| | C13D1 until end | | | | | | |
| | of study treatment | | | | | | |
| | First 2 Follow-up visits | | | X | X | Xa | Xa |
| | (approximately up to | | | | | | |
| | 100 days from the | | | | | | |
| | discontinuation | | | | | | |
| | of study drug | | | | | | |

^{a}Part A indicates first 12 weeks of treatment (nivolumab + ipilimumab dosing). Part B indicates nivolumab monotherapy period starting from Week 13
^{b}If a subject permanently discontinues both study drug treatments during the sampling period, they will move to sampling at the follow up visits. If a subject discontinues during the initial 4 doses when nivolumab and ipilimumab are administered together, follow-up samples should be collected for both the drugs. If a subject discontinues during the nivolumab monotherapy phase, follow up samples should be collected only for nivolumab
^{c}Samples must be collected before the administration of the first drug

TABLE 15
Pharmacokinetic (PK) and Immunogenicity Sample Collections-
Nivolumab + Ipilimumab Arm C

| | | Time | Pharmaco- | Immuno- | Pharmaco- | Immuno- |
|---|---|---|---|---|---|---|
| | Event | (Relative | kinetic | genicity | kinetic | genicity |
| | (Relative | To | Blood | Blood | Blood | Blood |
| Study Day^{a} | To | Dosing) | Sample | Sample | Sample | Sample |
| (1 Cycle = | Dosing) | Hour: | for | for | for | for |
| 2 weeks) | Hour | Min | Nivolumab | Nivolumab | Ipilimumab | Ipilimumab |
| C1D1 | Predose^{b} | 00:00 | X | X | X | X |
| (Ipilimumab | | | | | | |
| dose 1) | | | | | | |
| C4D1 | Predose^{b} | 00:00 | X | X | X | X |
| (Ipilimumab | | | | | | |
| dose 2) | | | | | | |
| C10D1 | Predose^{b} | 00:00 | X | X | X | X |
| (Ipilimumab | | | | | | |
| dose 4) | | | | | | |
| D1 of every | Predose^{b} | 00:00 | X | X | X | X |
| 9th cycle after | | | | | | |
| C10D1 until | | | | | | |
| end of study | | | | | | |
| treatment (or | | | | | | |
| Ipilimumab | | | | | | |
| Dose 7, 10, | | | | | | |
| 13. . . etc.) | | | | | | |
| First 2 Follow- | | | X | X | X | X |
| up visits | | | | | | |
| (approximately | | | | | | |
| up to 100 days | | | | | | |
| from the | | | | | | |
| discontinuation | | | | | | |
| of study drug | | | | | | |

^{a}If a subject permanently c is continues both study drug treatments during the sampling period, they will move to sampling at the follow up visits.
If ipilimumab is discontinued and nivolumab continues, ipilimumab PK and ADA should be collected only for the next 2 time points (corresponding to nivolumab sample collection) according to the PK table.
If nivolumab is discontinued and ipilimumab continues, nivolumab PK and ADA should becollected only for the next 2 time points (corresponding to ipilimumab sample collection) according to the PK table
^{b}Samples must be collected before the administration of the drug

### Pharmacokinetic and Immunogenicity Collection and Processing

A detailed schedule of PK and immunogenicity evaluations is provided in Table 13 to Table 15. PK samples will be analyzed for nivolumab/ipilimumab by a validated ligand binding assay. Immunogenicity samples will be analyzed for anti-nivolumab antibodies/anti-ipilimumab antibodies by a validated immunogenicity assay; samples may also be analyzed for neutralizing antibodies by a validated method. Serum samples may be analyzed by an exploratory method that measures anti-drug antibodies for technology exploration purposes; exploratory results will not be reported. Serum samples designated for PK or biomarker assessments may also be used for immunogenicity analysis if required (e.g., insufficient volume for complete immunogenicity assessment or to follow up on suspected immunogenicity related AE).

### Biomarker Assessments

Tumor Tissue Specimens

Archival (or fresh) FFPE tumor tissue collected within 6 months prior to enrollment must be sent to a third party laboratory for determination of PD-L1 status using the analytically validated IHC assay. PD-L1 stained tissue samples will be assessed by a pathologist at a central lab identified by the Sponsor and scored as PD-L1+ if membrane staining is observed in greater than or equal to 5% tumor cells among a minimum of 100 evaluable tumor cells. Tissue will also be analyzed by IHC to determine the abundance of immunoregulatory proteins such as, but not limited to, PD-L1, PD-L2, PD-1, and other markers associated with TILs (e.g., CD4, CD8, FOXP3). These data will be evaluated for associations with clinical endpoints.

FFPET may be evaluated also by FISH, genetic mutation detection methods, immunophenotyping and/or by QPCR for exploratory analyses of prognostic or predictive molecular markers associated with NSCLC (e.g., gene mutation, amplification or overexpression), or to determine if these factors influence response to nivolumab.

Peripheral Blood Markers

A variety of factors that may impact the immunomodulatory properties and efficacy of nivolumab will be investigated in peripheral blood specimens taken from all subjects prior to or during treatment. Data from these investigations will be evaluated for associations with response, survival, and/or safety (adverse event) data. Several analyses will be completed and are described briefly below.

Single Nucleotide Polymorphisms (SNPs)

Whole blood will be collected from all subjects prior to treatment to generate genomic DNA for Single Nucleotide Polymorphism (SNP) analyses. These analyses will focus on SNPs within genes associated with PD1 and other immunoregulatory signaling pathways to determine if natural variation within those genes is associated with response to nivolumab and/or with adverse events during treatment.

Serum Soluble Factors

To understand the prevalence of circulating proteins and the impact they may have on the clinical activity of nivolumab, the protein concentrations of a panel of cytokines, chemokines, and other relevant immunomodulatory, serum-soluble factors (e.g., soluble PD-L1) will be investigated at baseline and during treatment.

Serum MicroRNA (miRNA)

MicroRNAs (miRNA) are widely-expressed, small RNAs that regulate the abundance of mRNA transcripts and their translation into protein. Global miRNA expression profiling has become increasingly common in cancer research, and miRNA signatures that are correlated to stage of disease or to clinical outcomes are now available for a variety of cancer types. Expression profiling of miRNA may be useful also in identifying molecular markers for the prediction of drug-responses and for prospective stratification. Intriguingly, miRNAs are stable in serum and may represent miRNAs over-expressed in tumors and/or reflect immune system activity. Serum taken at baseline and during treatment from subjects randomized to each treatment arm will be analyzed for miRNA content by microarray or similar methodology. The resulting miRNA profiles will be evaluated for changes in miRNA abundance that occurs following treatment and for associations with response and survival data. Ultimately, the goal will be to determine if unique, immune-relevant and/or NSCLC-relevant miRNA signatures exist and if they are potentially useful for identifying patients who are likely (or unlikely) to respond to nivolumab treatment.

Myeloid Derived Suppressor Cells (MDSC)

Myeloid derived suppressor cells are an immune cell population capable of suppressing T cell activation and proliferation. Low pre-treatment MDSC levels in peripheral blood may be associated with better overall survival in melanoma patients treated with the immunotherapeutic agent ipilimumab. MDSCs will be measured at baseline and on-treatment to assess pharmacodynamic changes or associations with outcome.

Peripheral Blood Mononuclear Cells (PBMCs)

At sites in the USA and Canada only, peripheral blood mononuclear cells in whole blood taken from subjects at baseline and on treatment and will be analyzed by flow cytometry or other methods (e.g., ELIspot) to assess immune cell activity.

Tumor Biopsy Gene Expression Profiling

As feasible, an optional fresh biopsy at baseline or at any time following initiation of treatment may be collected. RNA derived from either fresh tumor tissue samples or from laser capture microdissected cells within the FFPE tumor tissue samples will be examined for gene expression by Affymetrix gene array technology, quantitative RT-PCR or other high throughput profiling technology to detect expression of immune related genes in tumor cells and/or the tumor microenvironment.

Tumor tissue or derived RNA/DNA from these specimens may be evaluated also by FISH, genetic mutation detection methods, immunophenotyping and/or by QPCR for exploratory analyses of prognostic or predictive molecular markers associated with NSCLC (e.g., gene mutation, amplification or overexpression), or to determine if these factors influence response to nivolumab.

### Outcomes Research Assessments

The evaluation of health related quality of life is an increasingly important aspect of a clinical efficacy. Such data provides an understanding of the impact of treatment from the subjects' perspective and offers insights into the patient experience that may not be captured through physician reporting. Generic health related quality of life scales additionally provide data necessary in calculating utility values for health economic models. The EQ-5D will be collected in order to assess the impact of study treatment on generic health related quality of life, which will also be used in populating health economic models most notably, cost effectiveness analysis.

The Lung Cancer Symptom Scale (LCSS) will be collected to assess the impact of study treatment on patient reported disease related symptoms. The Lung Cancer Symptom Scale is a validated instrument designed to assess the impact of treatment on disease-related symptoms. It consists of 6 symptom specific questions related to dyspnea, cough, fatigue, pain, hemoptysis and anorexia plus 3 summary items: symptom distress, interference with activity, and global health related quality of life (HRQoL). The degree of impairment is recorded on a 100 mm visual analogue scale with scores from 0 to 100 with zero representing the best score.

General health status will be measured using the EQ-5D. The EQ-5D is a standardized instrument for use as a measure of self-reported health status. The EQ-5D comprises 5 dimensions (mobility, self-care, usual activities, pain/discomfort, and anxiety) and a visual analog rating scale (VAS). The utility data generated from the EQ-5D is recommended for and commonly used in cost effectiveness analysis.

### Immunogenicity Assessments

Blood samples for immunogenicity analyses of nivolumab and ipilimumab will be collected according to the schedule given in Tables 13-15. Samples collected from subjects in each treatment arm will be evaluated for development of Anti-Drug Antibody (ADA) for nivolumab/ipilimumab by validated immunoassays. Samples may also be analyzed for neutralizing ADA response to nivolumab/ipilimumab.

### Adverse Events

An Adverse Event (AE) is defined as any new untoward medical occurrence or worsening of a preexisting medical condition in a clinical investigation subject administered study drug and that does not necessarily have a causal relationship with this treatment. An AE can therefore be any unfavorable and unintended sign (such as an abnormal laboratory finding), symptom, or disease temporally associated with the use of study drug, whether or not considered related to the study drug.

The causal relationship to study drug is determined by a physician and should be used to assess all adverse events (AE). The causal relationship can be one of the following:

Related: There is a reasonable causal relationship between study drug administration and the AE.

Not related: There is not a reasonable causal relationship between study drug administration and the AE.

The term “reasonable causal relationship” means there is evidence to suggest a causal relationship.

A Serious Adverse Event (SAE) is any untoward medical occurrence that at any dose: results in death; is life-threatening (defined as an event in which the subject was at risk of death at the time of the event; it does not refer to an event which hypothetically might have caused death if it were more severe); requires inpatient hospitalization or causes prolongation of existing hospitalization; results in persistent or significant disability/incapacity; is a congenital anomaly/birth defect; is an important medical event (defined as a medical event(s) that may not be immediately life-threatening or result in death or hospitalization but, based upon appropriate medical and scientific judgment, may jeopardize the subject or may require intervention [e.g., medical, surgical] to prevent one of the other serious outcomes listed in the definition above.) Examples of such events include, but are not limited to, intensive treatment in an emergency room or at home for allergic bronchospasm; blood dyscrasias or convulsions that do not result in hospitalization.) Potential drug induced liver injury (DILI) is also considered an important medical event; Suspected transmission of an infectious agent (e.g., pathogenic or nonpathogenic) via the study drug is an SAE.

Although pregnancy, overdose, cancer, and potential drug induced liver injury (DILI) are not always serious by regulatory definition, these events must be handled as SAEs.

The following hospitalizations are not considered SAEs in BMS clinical studies: a visit to the emergency room or other hospital department <24 hours, that does not result in admission (unless considered an important medical or life-threatening event); elective surgery, planned prior to signing consent; admissions as per protocol for a planned medical/surgical procedure; routine health assessment requiring admission for baseline/trending of health status (e.g., routine colonoscopy); medical/surgical admission other than to remedy ill health and planned prior to entry into the study; admission encountered for another life circumstance that carries no bearing on health status and requires no medical/surgical intervention (e.g., lack of housing, economic inadequacy, caregiver respite, family circumstances, administrative reason); or Admission for administration of anticancer therapy in the absence of any other SAEs (applies to oncology protocols).

Potential Drug Induced Liver Injury (DILI)

Potential drug induced liver injury is defined as:

1. AT (ALT or AST) elevation >3 times upper limit of normal (ULN) AND

2. Total bilirubin >2 times ULN, without initial findings of cholestasis (elevated serum alkaline phosphatase),

AND

3. No other immediately apparent possible causes of AT elevation and hyperbilirubinemia, including, but not limited to, viral hepatitis, pre-existing chronic or acute liver disease, or the administration of other drug(s) known to be hepatotoxic.

### Sample Size Determination

The sample size is calculated to compare OS between nivolumab and platinum doublet chemotherapy, and to compare OS between nivolumab in combination with ipilimumab and platinum doublet chemotherapy, at a Type I error level of 0.0167 (two-sided) and 90% power for each comparison. The number of events and power are calculated assuming an exponential distribution in platinum doublet chemotherapy arm and a piecewise mixture distribution in each of the experimental treatment arms. Table 16 summarizes the key parameters of the overall survival analysis.

TABLE 16
Parameters of the Overall Survival Analysis

| | Type-I error/Power | Sample size | Timing |
|---|---|---|---|
| Platinum doublet | α = 0.0167; Power = | 1200 subjects | 257 events in the |
| chemotherapy arm (Arm D): | 90% per pair-wise | | platinum doublet |
| Exponential distribution | comparison | | chemotherapy arm |
| with median OS = 13 months | | | (Arm D) 1 at 48 |
| Experimental treatment | | | months (14 months |
| arms (Arm A, Arm B and | | | of accrual + 34 |
| Arm C): Piecewise mixture | | | months of follow-up) |
| distribution with median | | | |
| OS = 18 months | | | |

Results from ipilimumab phase-III studies (Hodi F S, et al., N Eng J Med. 2010; 363: 711-723; Robert C, et al., N Eng J Med. 2011; 364: 2517-2526) in metastatic melanoma patients have demonstrated long term survival benefits in patients treated with ipilimumab observed as long lasting plateau towards the tail of survival curve. Results also suggested a delayed effect observed as late separation of survival curves between experimental and control arms of the studies. Both long-term survival and delayed onset of benefit may be particular to immuno-oncology drugs based on their mechanisms of action.

Exponential distribution assumption for OS in platinum doublet chemotherapy arm is made based on the considerations of a mixture of subjects with squamous and non-squamous NSCLC. In addition, consideration has also been given to that possibility that patients in platinum doublet chemotherapy arm may receive second line nivolumab (or another anti PD-1 agent) post progression. It is estimated that approximately 30% of subjects from the platinum doublet arm may receive second line anti-PD-1 therapy. The actual rate will be closely monitored to enable necessary adjustment to the number of events.

This study assumes an exponential OS distribution for platinum doublet therapy arm (13 months median OS) and a piecewise mixture distribution for each of the experimental treatment arms with an 18 months median OS. Power calculations were performed via simulations conducted using Power Analysis & Sample Size Software® (PASS).56 Additional details on power calculations will be documented in statistical analysis plan.

Approximately 1200 subjects will be randomized to the 4 treatment groups in a 1:1:1:1 ratio. The final analysis will be conducted after 257 events occur in the control group, and these events will be monitored by the un-blinded independent statistician supporting the DMC. Assuming a 20% screening failure rate, it is estimated that approximately 1500 subjects will be enrolled in order to have 1200 subjects randomized. assuming a piecewise constant accrual rate (8 subjects/month during Month 1 to 2, 40 subjects/month during Month 3 to 4, 85 subjects/month during Month 5 to 6, 138 subjects/month during Month 7 to 8, 170 subjects/month after Month 8), it will take approximately 48 months to obtain the required number of death for the final OS analysis (14 months for accrual and 34 months for survival follow up).

### Endpoints

A primary objective will be measured by the endpoint of OS in all randomized subjects. It is defined as the time between the date of randomization and the date of death due to any cause. OS will be censored on the last date a subject was known to be alive. OS will be followed continuously while subjects are on the study drug and every 3 months after subjects discontinue the study drug.

Progression-free Survival (PFS) is defined as the time between the date of randomization and the first date of documented progression, as determined by BICR, or death due to any cause, whichever occurs first. Subjects who die without a reported progression will be considered to have progressed on the date of their death. Subjects who did not progress or die will be censored on the date of their last evaluable tumor assessment. Subject who did not have any on study tumor assessments and did not die will be censored on their date of randomization. Subjects who had palliative local therapy or initiated anti-cancer therapy without a prior reported progression will be censored on the date of their last evaluable tumor assessment prior to the initiation of subsequent anti-cancer therapy or palliative local therapy. Tumor assessments are scheduled to be performed at Week 6 (±7 days), every 6 Weeks until Week 48 (±7 days) and then every 12 Weeks (±7 days) until disease progression or treatment discontinuation, whichever occurs later.

Objective Response Rate (ORR) is defined as the number of subjects with a BOR of CR or PR divided by the number of randomized subjects for each treatment group. BOR is defined as the best response designation, recorded between the date of randomization and the date of objectively documented progression per RECIST 1.1 as determined by BICR or the date of initiation of palliative local therapy or the date of subsequent anti-cancer therapy, whichever occurs first. For subjects without documented progression or palliative local therapy or subsequent anti-cancer therapy, all available response designations will contribute to the BOR determination. For subjects who continue study medication beyond progression, the BOR should be determined based on response designations recorded at the time of the initial RECIST 1.1 defined progression.

PD-L1 Protein Expression is defined as the percent of tumor cells demonstrating plasma membrane PD-L1 staining of any intensity using the validated DAKO PD-L1 IHC assay.

Disease-Related Symptom Improvement Rate by week 12 is defined as the proportion of subjects who had a disease-related symptom improvement as measured by the LCSS at any time between randomization and week 12 among all randomized subject. The first six items of the LCSS are summarized into a symptom scale ranging in score from zero to one hundred, with zero being the best possible score and one hundred the worst possible score. The minimum important change in the LCSS used to define symptom improvement is approximately a change of 10 mm in a Visual Acuity Scale (VAS), and that definition has been used for this NSCLC symptom scale in other trials. Disease-related symptom improvement is defined as a subject decreasing by 10 mm on the average LCSS VAS relative to the subject's baseline average LCSS score. LCSS questionnaire is completed on Day 1 of the scheduled cycle for the first 6 months on study treatment, then every 6 weeks thereafter for the remainder of the study, and at the first two follow-up visits. See Section 5.1 for frequency of assessments on study for each arm.

Safety and tolerability objective will be measured by the incidence of adverse events, serious events, deaths, and laboratory abnormalities.

Adverse event assessment and laboratory tests are performed at baseline, and continuously throughout the study at the beginning of each subsequent cycle.

The PK objective will be measured from serum concentration. Samples will be collected to characterize pharmacokinetics of nivolumab and to explore exposure-safety and exposure-efficacy relationships.

Each of the three primary OS analyses will be conducted using a two-sided log-rank test stratified by histology and PD-L1 status in all randomized subjects using Hochberg's procedure to address multiplicity. Hazard ratios (HR) and corresponding two-sided (1-adjusted α) % confidence intervals (CI) will be estimated using a Cox proportional hazard model, with treatment group as a single covariate, stratified by the above factors. OS curves, OS medians with 95% CIs, and OS rates at 12 and 24 months with 95% CIs will be estimated using Kaplan-Meier methodology.

If OS superiority is demonstrated for at least one comparison, a gatekeeping testing approach for the key secondary endpoints will be applied to additional experimental vs. control comparisons as described in the statistical analysis plan. The alpha level retained for testing of secondary endpoints will depend on the positive OS comparison and ensure that the overall type I error is adequately maintained. The key secondary endpoints will be tested in the following hierarchical order:

1) PFS (based on BICR assessments) analyses will be conducted using a two-sided log-rank test stratified by histology and PD-L1 status in all randomized subjects to compare each of the three experimental treatments to the control group. HRs and corresponding two-sided (1-adjusted α) % CIs will be estimated using a Cox proportional hazard model, with treatment group as a single covariate, stratified by the above factors. PFS curves, PFS medians with 95% CIs, and PFS rates at 6 and 12 months with 95% CIs will be estimated using Kaplan-Meier methodology.

2) ORR (based on BICR assessments) analyses will be conducted using a two-sided Cochran-Mantel-Haenszel (CMH) test stratified by PD-L1 status and histology to compare each of the three experiment treatments to the control group. Associated odds ratios and (1-adjusted α) % CI will also be calculated. Additionally, ORRs and their corresponding 95% exact CIs will be calculated using the Clopper-Pearson method for each of the four treatment groups.

3) Pairwise comparison of OS among experimental arms will be conducted using a two-sided log-rank test stratified by histology and PD-L1 status. HRs and corresponding two-sided (1-adjusted α) % CIs will be estimated using a Cox proportional hazard model, with treatment group as a single covariate, stratified by the above factors.

Descriptive analyses of PFS and ORR will be performed to evaluate differences between nivolumab monotherapy and nivolumab in combination of ipilimumab groups. These include HRs and medians with corresponding two-sided 95% CIs for PFS, as well as an ORR odds ratio with corresponding 95% CI.

Analyses of PD-L1 expression will be descriptive. Distribution of PD-L1 expression will be examined based on overall population. Potential associations between PD-L1 expression and efficacy measures (ORR, OS, PFS) will be assessed. If there is an indication of a meaningful association, future work will evaluate PD-L1 expression as a predictive biomarker, including selection of an optimal PD-L1 expression cut-off to classify subjects as PD-L1 positive or PD-L1 negative. Cut-off selection and validation will be conducted across studies.

Safety analysis will be performed in all treated subjects. Descriptive statistics of safety will be presented using National Cancer Institute (NCI) Common Terminology Criteria for Adverse Events (CTCAE) version 4.0 by treatment group. All on-study AEs, drug-related AEs, SAEs and drug-related SAEs will be tabulated using worst grade per NCI CTCAE v 4.0 criteria by system organ class and preferred term. On-study lab parameters including hematology, coagulation, chemistry, liver function and renal function will be summarized using worst grade per NCI CTCAE v 4.0 criteria.

The nivolumab concentration data obtained in this study may be combined with data from other studies in the clinical development programs to develop or refine a population PK model. These models may be used to evaluate the effects of intrinsic and extrinsic covariates on the PK of nivolumab and ipilimumab to determine measures of individual exposure. In addition, model determined exposures of nivolumab and ipilimumab may be used for exposure-response analyses with efficacy and safety measures in combination. Results of population PK and exposure-response analyses will be reported separately.

Methodology for exploratory biomarker analyses is described herein.

Outcome research analysis will be performed based on all randomized subjects. LCSS questionnaire complete rate, defined as the proportion of questionnaires actually received out of the expected number (i.e., the number of subjects still on treatment in follow-up), will be calculated and summarized at each assessment point.

The disease-related symptom improvement rate at week 12 and its corresponding 95% exact CI will also be calculation by Clopper-Pearson method for each randomized arm. Baseline and change from baseline of the average symptom burden scale index score at each assessment point will be summarized using descriptive statistics (N, mean, median, SD) by treatment group as randomized.

The EQ-5D will be used to assess the subject's overall health status. EQ-5D essentially has 2 components-the EQ-5D descriptive system and the EQ visual analogue scale (EQ VAS). The EQ-5D descriptive system comprises the following 5 dimensions: mobility, self-care, usual activities, pain/discomfort and anxiety/depression. Each dimension has 3 levels: no problems, some problems, sever problems. The EQ VAS records the subject's self-rated health state on a 100-point vertical, visual analogue scale (0=worst imaginable health state, 100=best imaginable health state).

Subject's overall health state on a visual analog scale (EQ-VAS) at each assessment time point will be summarized using descriptive statistics by treatment group, as randomized.

Proportion of subjects reporting problems for the 5 EQ-5D dimensions at each assessment time point will be summarized by level of problem and by treatment group, as randomized. Percentages will be based on number of subjects assessed at assessment time point.

Summary statistics will be calculated for the population preference-based health state utility score (EQ-5D index).

Methodology for exploratory analyses including immunogenicity is described herein.

The present invention is further illustrated by the following examples which should not be construed as further limiting. The contents of all references cited throughout this application are expressly incorporated herein by reference.

### Example 1

Treatment of NSCLC with Nivolumab Monotherapy or Nivolumab+Ililimumab v. Platinum Doublet Chemotherapy

In a phase 3 CA209-227 study, treatment with nivolumab monotherapy or nivolumab combined with ipilimumab is tested to determine if there is an improvement in overall survival (OS) compared to platinum doublet chemotherapy in chemotherapy-naive subjects with stage IV or recurrent NSCLC. A formal pairwise comparison of OS among experimental arms is conducted.

The study also compares the progression-free survival (PFS) and the objective response rate (ORR), based on Blinded Independent Central Review (BICR) assessment of nivolumab monotherapy and nivolumab in combination with ipilimumab, to platinum-doublet chemotherapy in subjects with previously untreated stage IV or recurrent NSCLC. Differences in PFS and ORR between nivolumab combined with ipilimumab and nivolumab monotherapy in subjects with stage IV or recurrent NSCLC are evaluated.

The study also evaluates whether PD-L1 expression is a predictive biomarker for OS or PFS. And evaluates the proportion of treated patients exhibiting disease-related symptom improvement by 12 weeks as measured by the Lung Cancer Symptom Score (LCSS) in subjects receiving nivolumab monotherapy, nivolumab in combination with ipilimumab and in subjects receiving platinum doublet chemotherapy.

Other objectives of the study include: 1) assessing the overall safety and tolerability of nivolumab and nivolumab in combination with ipilimumab compared to platinum-doublet chemotherapy; 2) characterizing pharmacokinetics of nivolumab in combination with ipilimumab and explore exposure-safety and exposure-efficacy relationships; 3) characterizing immunogenicity of nivolumab in combination with ipilimumab; 4) characterizing immune correlates of nivolumab, nivolumab in combination with ipilimumab and platinum-doublet chemotherapy; 5) assessing predictive tumor and peripheral biomarkers of clinical response to nivolumab and nivolumab in combination with ipilimumab; and 6) assessing overall health status using the EQ-5D index and visual analogue scale in subjects treated with nivolumab in combination with ipilimumab and in those treated with platinum doublet chemotherapy.

### Methods

### Study Design

The study is an open label 4-arm, randomized, Phase 3 study in adult (greater than or equal to 18 years of age) male and female subjects, with stage IV or recurrent non-small cell lung cancer (NSCLC), PD-L1 positive or negative, previously untreated for advanced disease.

Key inclusion criteria include: 1) ECOG Performance Status of greater than or equal to 1 (See Table 7); 2) Patients with histologically confirmed Stage IV or recurrent NSCLC (per the 7th International Association for the Study of Lung Cancer classification squamous or non-squamous histology), with no prior systemic anticancer therapy (including EGFR and ALK inhibitors) given as primary therapy for advanced or metastatic disease; and 3) Measurable disease by CT or MRI per RECIST 1.1 criteria.

Key exclusion criteria include: 1) Subjects with known EGFR mutations which are sensitive to available targeted inhibitor therapy; 2) Subjects with known ALK translocations which are sensitive to available targeted inhibitor therapy; 3) Subjects with untreated CNS metastases; 4) Subjects with an active, known or suspected autoimmune disease (subjects with type I diabetes mellitus, hypothyroidism only requiring hormone replacement, skin disorders (such as vitiligo, psoriasis, or alopecia) not requiring systemic treatment, or conditions not expected to recur in the absence of an external trigger are permitted to enroll); and 5) Subjects with a condition requiring systemic treatment with either corticosteroids (>10 mg daily prednisone equivalent) or other immunosuppressive medications within 14 days of randomization (inhaled or topical steroids, and adrenal replacement steroid >10 mg daily prednisone equivalent, are permitted in the absence of active autoimmune disease).

Subjects are randomized 1:1:1:1, and stratified by histology (Squamous versus Non-squamous) and PD-L1 status. PD-L1 status is determined by immunohistochemical (IHC) staining of PD-L1 protein in a tumor sample submitted prior to randomization. Subjects are identified as PD-L1 positive if greater than or equal to 5% tumor cell membrane staining in a minimum of a hundred evaluable tumor cells is observed, or PD-L1 negative if less than 5% tumor cell membrane staining in a minimum of a hundred evaluable tumor cells is observed.

Subjects receive open-label treatment from one of four study arms. The dosing schedule is shown in Table 17. The maintenance schedule for nivolumab (Arm B) and optional pemetrexed (Arm D) is shown in Table 18.

TABLE 17
Dosing Schedule*

| | Week 1 | Week 2 | Week 3 | Week 4 | Week 5 | Week 6 |
|---|---|---|---|---|---|---|
| Arm A: | Day 1 | | Day 1 | | Day 1 | |
| Nivolumab 240 mg^{a} | Nivolumab | | Nivolumab | | Nivolumab | |
| q 2 weeks | | | | | | |
| Arm B: | Day 1 | | | Day 1 | | |
| Nivolumab 1 mg/kg + | Nivolumab + | | | Nivolumab + | | |
| Ipilimumab | Ipilimumab | | | Ipilimumab | | |
| 1 mg/kg q 3weeks × | | | | | | |
| 4^{a} followed by | | | | | | |
| maintenance | | | | | | |
| nivolumab | | | | | | |
| Arm C: | Day 1 | | Day 1 | | Day 1 | |
| Nivolumab 3 mg/kg | Nivolumab + | | Nivolumab | | Nivolumab | |
| q 2 weeks^{a} + | Ipilimumab | | | | | |
| Ipilimumab | | | | | | |
| 1 mg/kg q | | | | | | |
| 6 weeks | | | | | | |
| Arm D | Day 1: | Day 8 | | Day 1: | Day 8 | |
| Platinum doublet | Gemcitabine/ | Gemcitabine | | Gemcitabine/ | Gemcitabine | |
| chemotherapy: | Cisplatin or | | | cisplatin or | | |
| q 3 weeks × 4-6 | Gemcitabine/ | | | Gemcitabine/ | | |
| followed by optional | Carboplatin or | | | carboplatin or | | |
| maintenance | Pemetrexed/ | | | Pemetrexed/ | | |
| Pemetrexed for | Cisplatin or | | | cisplatin or | | |
| nonsquamous | Pemetrexed/ | | | Pemetrexed/ | | |
| histology | Carboplatin | | | carboplatin | | |

*Both nivolumab and ipilimumab may be administered as 30 minute infusions
^{a}continues until disease progression, discontinuation due to unacceptable toxicity, withdrawal of consent, or study closure

TABLE 18
Maintenance Schedule for Nivolumab (Arm B) and Optional Pemetrexed (Arm D)

| | Week 1 | Week 2 | Week 3 | Week 4 | Week 5 | Week 6 |
|---|---|---|---|---|---|---|
| Arm B: | Day 1 | | Day 1 | | Day 1 | |
| Nivolumab | Nivolumab | | Nivolumab | | Nivolumab | |
| 3 mg/kg | | | | | | |
| q 2 weeks a | | | | | | |
| Arm D: | Day 1 | | | Day 1 | | |
| (pemetrexed | Pemetrexed | | | Pemetrexed | | |
| 500 mg/m²) | | | | | | |

a continues until disease progression, discontinuation due to unacceptable toxicity, withdrawal of consent, or study closure.

On-study tumor assessment begins at Week 6 post randomization (±7 days) and is performed every 6 weeks (±7 days) until Week 48. After Week 48, tumor assessment is performed every 12 weeks (±7 days) until progression or treatment discontinuation, whichever occurs later. Subjects receiving nivolumab or nivolumab plus ipilimumab beyond investigator-assessed RECIST 1.1-defined progression must also continue tumor assessments until such treatment is discontinued. Enrollment will end after approximately 1200 subjects are randomized. The primary endpoint of the study is Overall Survival (OS). The duration of the study from start of enrollment to analysis of the primary OS endpoint is expected to be approximately 48 months.

The study design schematic is presented in FIG. 1.

### Study Arms

### Nivolumab Monotherapy (Arm A)

Nivolumab 240 mg is administered intravenously (IV) on day 1 of each cycle over 30 minutes every 2 weeks until disease progression, discontinuation due to unacceptable toxicity, withdrawal of consent or study closure. Treatment beyond initial investigator-assessed RECIST 1.1-defined progression is permitted if the subject has investigator-assessed clinical benefit and is tolerating treatment. Upon completion of dosing, subjects enter the Follow-up Phase.

Study assessments are collected as outlined in Table 19.

TABLE 19
On Study Assessments Treatment Phase-Arms A (CA209227)^{a}

| | | Each | | | |
|---|---|---|---|---|---|
| | | Subsequent | Every 2 | Every 3 | Notes |
| | Cycle 1 | Cycle | Cycles | Cycles | For purposes of this table, a cycle refers to the |
| Procedure | Day 1 | Day 1 | Day 1 | Day 1 | nivolumab every 2 weeks regimen. |
| Safety Assessments | | | | | |
| Physical | X | X | | | See, e.g., Table 7 |
| Measurements & | | | | | |
| ECOG Performance | | | | | |
| Status | | | | | |
| Vital Signs and | | X | | | |
| Oxygen Saturation | | | | | |
| Adverse Event | Continuously during the study | | | | |
| Assessments | | | | | |
| Review of | X | X | | | |
| Concomitant | | | | | |
| Medications | | | | | |
| Laboratory Tests | X | X | | X (TSH) | Within 72 hrs prior to dosing to include CBC w/ |
| | | | | | differential, AST, ALT, ALP, T. Bili, BUN or serum urea |
| | | | | | level, creatinine, albumin, Ca, Mg, Na, K, Cl, LDH, |
| | | | | | glucose, amylase, lipase, TSH (with reflexive Free T4 |
| | | | | | and Free T3). |
| | | | | | Thyroid Function Testing to be evaluated every 6 weeks |
| | | | | | Note: C1D1 labs do not need to be repeated if they were |
| | | | | | performed within 14 days of dosing. |
| Pregnancy Test | X | | X | | To be evaluated at least every 4 weeks |
| (WOCBP only) | | | | | |
| Efficacy Assessments | | | | | |
| Radiographic Tumor | FIRST tumor assessment should first be performed at 6 weeks (±7 days) from randomization date. | | | | |
| Assessment | SUBSEQUENT tumor assessments should occur every 6 weeks (±7 days) up to first 12 months (week 48), then | | | | |
| (CT/MRI of chest, | every 12 weeks until disease progression. | | | | |
| abdomen, pelvis) | * Subjects with a history of brain metastasis may have surveillance MRI approximately every 12 weeks from the | | | | |
| | date of first dose, or sooner if clinically indicated. | | | | |
| Exploratory Biomarker | | | | | |
| Assessments | | | | | |
| Single Nucleotide | X | | | | Obtained prior to dosing. |
| Polymorphisms | | | | | |
| (SNPs) | | | | | |
| Serum for Soluble | C1D1 pre-dose, C1D1 post-dose, C2D1, | | | Obtained prior to dosing | |
| Factors and miRNA | C3D1, C4D1, C5D1 | | | | |
| Analyses | | | | | |
| Myeloid Derived | X | | | | Obtained prior to dosing. |
| Suppressor Cells | | | | | |
| (MDSCs) | | | | | |
| Peripheral Blood | C1D1 pre-dose, C1D1 post-dose, C2D1, | | | Collected in USA and Canada Only. | |
| Mononuclear Cells | C3D1, C4D1, C5D1 | | | Obtained prior to dosing. | |
| (PBMCs) | | | | | |
| Optional Tumor | X | | | As feasible obtained at baseline or any time on treatment | |
| Biopsy Gene | | | | | |
| Expression Profiling | | | | | |
| Pharmacokinetic (PK) | | | | | |
| and Immunogenicity | | | | | |
| Assessments | | | | | |
| PK Samples | Throughout the study | | | See, e.g., Table 13 | |
| Immunogenicity | Throughout the study | | | See, e.g., Table 13 | |
| Samples | | | | | |
| Patient Reported | X | X | | After 6 | For C1D1 - LCSS and EQ-5D assessments performed |
| Outcomes | | | | months | after randomization, PRIOR to first dose (day −3 to +1). |
| Assessment (PRO) | | | | | For on-study visits: Assessments (LCSS and EQ-5D) will |
| | | | | | be performed PRIOR to any study procedures and treatment. |
| | | | | | Assessments will be performed at each cycle on Day 1 |
| | | | | | for the first 6 months on study, then every 6 weeks |
| | | | | | thereafter for the remainder of the treatment period |
| Health Resource | | X | | | Except cycle 1. To include: concomitant medication |
| Utilization | | | | | collection |
| Clinical Drug | | | | | |
| Supplies | | | | | |
| IVRS Vial | X | X | | | Within 1 day prior to dosing |
| Assignment | | | | | |
| Nivolumab 240 mg | X | X | | | See, e.g., Table 16 |
| q 2 weeks^{b} | | | | | |

^{a}If a dose is delayed, the procedures scheduled for that same timepoint should be delayed to coincide with when the time point's dosing actually occur.
^{b}continues until disease progression, discontinuation due to unacceptable toxicity, withdrawal of consent, or study closure.

### Nivolumab Plus Ipilimumab (Arm B)

Nivolumab 1 mg/kg is administered IV over 30 minutes combined with ipilimumab 1 mg/kg administered IV over 30 minutes every 3 weeks for four cycles for induction, followed by nivolumab 3 mg/kg administered IV over 30 minutes every 2 weeks. Nivolumab 1 mg/kg and ipilimumab 1 mg/kg will both be administered on day 1 of each 3 week treatment cycle for four cycles. Following the 4th treatment cycle, nivolumab 3 mg/kg will be administered every 2 weeks until disease progression, unacceptable toxicity, withdrawal of consent, or study closure. Treatment beyond initial investigator-assessed RECIST 1.1-defined progression is permitted if the subject has investigator-assessed clinical benefit and is tolerating treatment. Upon completion of dosing, subjects enter the Follow-up Phase.

Study assessments are collected as outlined in Table 20.

TABLE 20
On Study Assessments Treatment Phase-Arms B (CA209227)^{a}

| | | | | | Notes |
|---|---|---|---|---|---|
| | | Each | | | For the purposes of this table, a cycle refers to the |
| | | Subsequent | Every 2 | Every 3 | nivolumab + ipilimumab every 3 weeks x4 |
| | Cycle 1 | Cycle | Cycles | Cycles | regimen until for the maintenance phase when it |
| Procedure | Day 1 | Day 1 | Day 1 | Day 1 | refers to nivolumab every 2 weeks. |
| Safety Assessments | | | | | |
| Physical | X | X | | | See, e.g., Table 7 |
| Measurements & | | | | | |
| ECOG Performance | | | | | |
| Status | | | | | |
| Vital Signs and | | X | | | |
| Oxygen Saturation | | | | | |
| Adverse Event | Continuously during the study | | | | |
| Assessments | | | | | |
| Review of | X | X | | | |
| Concomitant | | | | | |
| Medications | | | | | |
| Laboratory Tests | X | X | X(TSH) | | Within 72 hrs prior to dosing to include CBC w/ |
| | | | | | differential, AST, ALT, ALP, T. Bili, BUN or serum urea |
| | | | | | level, creatinine, albumin, Ca, Mg, Na, K, Cl, LDH, |
| | | | | | glucose, amylase, lipase, TSH (with reflexive Free T4 |
| | | | | | and Free T3). |
| | | | | | Thyroid Function Testing to be evaluated every 6 weeks |
| | | | | | Note: C1D1 labs do not need to be repeated if they were |
| | | | | | performed within 14 days of dosing. |
| Pregnancy Test | X | X | | | To be evaluated at least every 3 weeks |
| (WOCBP only) | | | | | |
| Efficacy Assessments | | | | | |
| Radiographic Tumor | FIRST tumor assessment should first be performed at 6 weeks (±7 days) from randomization date. | | | | |
| Assessment | SUBSEQUENT tumor assessments should occur every 6 weeks (±7 days) up to first 12 months (week 48), then | | | | |
| (CT/MRI chest, | every 12 weeks until disease progression. | | | | |
| abdomen, pelvis) | * Subjects with a history of brain metastasis may have surveillance MRI approximately every 12 weeks from the | | | | |
| | date of first dose, or sooner if clinically indicated. | | | | |
| Exploratory Biomarker | | | | | |
| Single Nucleotide | X | | | | Obtained prior to dosing. |
| Polymorphisms | | | | | |
| (SNPs) | | | | | |
| Serum for Soluble | C1D1 pre-dose, C1D1 post-dose, C2D1, C3D1, C5D1 | | | | Obtained prior to dosing. |
| Factors and miRNA | | | | | |
| Analyses | | | | | |
| Myeloid Derived | X | | | | Obtained prior to dosing. |
| Suppressor Cells | | | | | |
| (MDSCs) | | | | | |
| Peripheral Blood | C1D1 pre-dose, C1D1 post-dose, C2D1, C3D1, C5D1 | | | | Collected in USA and Canada Only. |
| Mononuclear Cells | | | | | Obtained prior to dosing. |
| (PBMCs) | | | | | |
| Optional Tumor | X | | | | As feasible obtained at baseline or any time on treatment |
| Biopsy for Gene | | | | | |
| Expression Profiling | | | | | |
| Pharmacokinetic (PK) | | | | | |
| and Immunogenicity | | | | | |
| Assessments | | | | | |
| PK Samples | Throughout the study | | | | See, e.g., Table 14 |
| Immunogenicity | Throughout the study | | | | See, e.g., Table 14 |
| Samples | | | | | |
| Patient Reported | X | X | | After 6 | For C1D1 - LCSS and EQ-5D assessments performed |
| Outcomes | | | | months | after randomization PRIOR to first dose (day −3 to +1). |
| Assessment (PRO) | | | | | For on-study visits: Assessments (LCSS and EQ-5D) will |
| | | | | | be performed PRIOR to any study procedures and treatment. |
| | | | | | Assessments will be performed at each cycle on Day 1 |
| | | | | | for the first 6 months on study, then every 6 weeks |
| | | | | | thereafter for the remainder of the treatment period |
| Health Resource | | X | | | Except cycle 1. To include: concomitant medication |
| Utilization | | | | | collection. |
| Clinical Drug | | | | | |
| Supplies | | | | | |
| IVRS Vial | X | X | | | Within 1 day prior to dosing |
| Assignment | | | | | |
| Nivolumab 1 mg/kg + | X | X | | | See, e.g., Table 16. |
| Ipilimumab 1 mg/kg | | | | | |
| q 3 w x4 | | | | | |
| Maintenance Phase | | | | | |
| Dose Schedule | | | | | |
| Nivolumab 3 mg/kg^{b} | X | X | | | See, e.g., Table 17. |

^{a}If a dose is delayed, the procedures scheduled for that same time point should be delayed to coincide with when the time point's dosing actually occur
^{b}continue until disease progression, discontinuation due to unacceptable toxicity, withdrawal of consent, or study closure

### Nivolumab Plus Ipilimumab (Arm C)

Nivolumab 3 mg/kg is administered IV over 30 minutes every 2 weeks, and ipilimumab 1 mg/kg is administered IV over 30 minutes at 1 mg/kg every 6 weeks following the administration of nivolumab until progression, discontinuation due to toxicity, withdrawal of consent, or study closure. Subjects may discontinue only one study drug and continue treatment with the other if certain circumstances are met. Treatment beyond initial investigator-assessed RECIST 1.1 defined progression is permitted if the subject has investigator assessed clinical benefit and is tolerating treatment. Upon completion of dosing, subjects enter the Follow-up Phase.

Study assessments are collected as outlined in Table 21.

TABLE 21
On Study Assessments Treatment Phase-Arms C (CA209227)^{a}

| | | Each | | | |
|---|---|---|---|---|---|
| | | Subsequent | Every 2 | Every 3 | Notes |
| | Cycle 1 | Cycle | Cycles | Cycles | For the purposes of this table, a cycle refers to |
| Procedure | Day 1 | Day 1 | Day 1 | Day 1 | nivolumab every 2 weeks. |
| Safety Assessments | | | | | |
| Physical | X | X | | | See, e.g., Table 7 |
| Measurements & | | | | | |
| ECOG Performance | | | | | |
| Status | | | | | |
| Vital Signs and | | X | | | |
| Oxygen Saturation | | | | | |
| Adverse Event | Continuously during the study | | | | |
| Assessments | | | | | |
| Review of | X | X | | | |
| Concomitant | | | | | |
| Medications | | | | | |
| Laboratory Tests | X | X | | X(TSH) | Within 72 hrs prior to dosing to include CBC w/ |
| | | | | | differential, AST, ALT, ALP, T. Bili, BUN or serum urea |
| | | | | | level, creatinine, albumin, Ca, Mg, Na, K, Cl, LDH, |
| | | | | | glucose, amylase, lipase, TSH (with reflexive Free T4 |
| | | | | | and Free T3). |
| | | | | | Thyroid Function Testing to be evaluated every 6 weeks |
| | | | | | Note: C1D1 labs do not need to be repeated if they were |
| | | | | | performed within 14 days of dosing. |
| Pregnancy Test | X | | X | | To be evaluated at least every 4 weeks |
| (WOCBP only) | | | | | |
| Efficacy Assessments | | | | | |
| Radiographic Tumor | FIRST tumor assessment should first be performed at 6 weeks (±7 days) from randomization date. | | | | |
| Assessment | SUBSEQUENT tumor assessments should occur every 6 weeks (±7 days) up to first 12 months (week 48), then | | | | |
| (CT/MRI of chest, | every 12 weeks until disease progression. | | | | |
| abdomen, pelvis) | * Subjects with a history of brain metastasis may have surveillance MRI approximately every 12 weeks from the | | | | |
| | date of first dose, or sooner if clinically indicated. | | | | |
| Exploratory Biomarker | | | | | |
| Assessments | | | | | |
| Single Nucleotide | X | | | | Obtained prior to dosing. |
| Polymorphisms | | | | | |
| (SNPs) | | | | | |
| Serum for Soluble | C1D1 pre-dose, C1D1 post-dose, C2D1, | | | | Obtained prior to dosing. |
| Factors and miRNA | C3D1, C4D1, C5D1 | | | | |
| Analyses | | | | | |
| Myeloid Derived | X | | | | Obtained prior to dosing. |
| Suppressor Cells | | | | | |
| (MDSCs) | | | | | |
| Peripheral Blood | C1D1 pre-dose, C1D1 post-dose, C2D1, | | | | Collected in USA and Canada Only. |
| Mononuclear Cells | C3D1, C4D1, C5D1 | | | | Obtained prior to dosing. |
| (PBMCs) | | | | | |
| Optional Tumor | X | | | | As feasible obtained at baseline or any time on treatment |
| Biopsy Gene | | | | | |
| Expression Profiling | | | | | |
| Pharmacokinetic (PK) | | | | | |
| and Immunogenicity | | | | | |
| Assessments | | | | | |
| PK Samples | Throughout the study | | | | See, e.g., Table13 |
| Immunogenicity | Throughout the study | | | | See, e.g., Table14 |
| Samples | | | | | |
| Patient Reported | X | X | | After 6 | For C1D1 - LCSS and EQ-5D assessments performed |
| Outcomes | | | | months | after randomization PRIOR to first dose (day −3 to +1). |
| Assessment (PRO) | | | | | For on-study visits: Assessments (LCSS and EQ-5D) will |
| | | | | | be performed PRIOR to any study procedures and treatment. |
| | | | | | Assessments will be performed at each cycle on Day 1 |
| | | | | | for the first 6 months on study, then every 6 weeks |
| | | | | | thereafter for the remainder of the treatment period |
| Health Resource | | X | | | Except cycle 1. To include: concomitant medication |
| Utilization | | | | | collection |
| Clinical Drug | | | | | |
| Supplies | | | | | |
| IVRS Vial | X | X | | | Within 1 day prior to dosing |
| Assignment | | | | | |
| Nivolumab 3 mg/kg | X | X | | | See, e.g., Table 16. |
| q 2 weeks^{b} + | | | | | |
| Ipilimumab 1 mg/kg | | | | | |
| q 6 weeks | | | | | |

^{a}If a dose is delayed, the procedures scheduled for that same timepoint should be delayed to coincide with when the time point's dosing actually occur
^{b}continues until disease progression, discontinuation due to unacceptable toxicity, withdrawal of consent, or study closure

### Platinum Doublet Chemotherapy and Optional Continuation Maintenance (Arm D)

Platinum-doublet chemotherapy is administered IV in 3-week cycles for up to a maximum of 6 cycles of chemotherapy. Chemotherapy treatment continues until disease progression, unacceptable toxicity or completion of the 4-6 cycles, whichever comes first. Platinum-doublet chemotherapy regimens are dependent on NSCLC histology. Subjects with mixed histology are classified according to the predominant histology.

Squamous histology subjects are given receive Gemcitabine (1250 mg/mg²) with cisplatin (75 mg/m²); or Gemcitabine (1000 mg/mg²) with carboplatin (AUC 5). Gemcitabine is administered on Day 1 and Day 8 of each cycle.

Non-squamous histology subjects receive Pemetrexed (500 mg/m²) with cisplatin (75 mg/m²), administered on Day 1 of each cycle; or Pemetrexed (500 mg/m²) with carboplatin (AUC 6), administered on Day 1 of each cycle.

Subjects with non-squamous histology who have stable disease or response after Cycle 4 are permitted to continue pemetrexed alone as maintenance therapy until disease progression or unacceptable toxicity. Upon completion of chemotherapy, subjects enter Follow-up Phase.

Study assessments are collected as outlined in Table 22.

TABLE 22
On Study Assessments Treatment Phase-Arms D (CA209227)^{a}

| | | Each | | | Notes |
|---|---|---|---|---|---|
| | | Subsequent | Every 2 | Every 3 | For purposes of this table, a cycle refers to |
| | Cycle 1 | Cycle | Cycles | Cycles | the platinum doublet chemotherapy every 3 |
| Procedure | Day 1 | Day 1 | Day 1 | Day 1 | weeks regimen. |
| Safety Assessments | | | | | |
| Physical | X | X | | | See, e.g., Table 17 |
| Measurements & | | | | | |
| ECOG Performance | | | | | |
| Status | | | | | |
| Vital Signs and | | X | | | |
| Oxygen Saturation | | | | | |
| Adverse Event | Continuously during the study | | | | Monitoring for adverse events related to |
| Assessments | | | | | chemotherapy drugs in Arm D subjects should |
| | | | | | follow recommendations specified in the local labels. |
| Review of | X | X | | | |
| Concomitant | | | | | |
| Medications | | | | | |
| Laboratory Tests | | X | X (TFTs) | | Within 72 hrs prior to dosing to include CBC w/ |
| | | | | | differential, AST, ALT, ALP, T. Bili, BUN or serum urea |
| | | | | | level, creatinine, albumin, Ca, Mg, Na, K, Cl, LDH, |
| | | | | | glucose, amylase, lipase, TSH (with reflexive Free T4 |
| | | | | | and Free T3). |
| | | | | | Thyroid Function Testing to be evaluated every 6 weeks. |
| | | | | | CBC required prior to gemcitabine dosing on Day 8 of |
| | | | | | each cycle. |
| | | | | | Note: C1D1 labs do not need to be repeated if they were |
| | | | | | performed within 14 days of dosing. |
| Pregnancy Test | X | X | | | To be evaluated at least every 3 weeks |
| (WOCBP only) | | | | | |
| Efficacy Assessments | | | | | |
| Radiographic Tumor | FIRST tumor assessment should first be performed at 6 weeks (±7 days) from randomization date. | | | | |
| Assessment | SUBSEQUENT tumor assessments should occur every 6 weeks (±7 days) up to first 12 months (week 48), then | | | | |
| (CT/MRI of chest, | every 12 weeks until disease progression. | | | | |
| abdomen, pelvis) | * Subjects with a history of brain metastasis may have surveillance MRI approximately every 12 weeks from the | | | | |
| | date of first dose, or sooner if clinically indicated. | | | | |
| Exploratory Biomarker | | | | | |
| Assessments | | | | | |
| Single Nucleotide | X | | | | Obtained prior to dosing. |
| Polymorphisms | | | | | |
| (SNPs) | | | | | |
| Serum for Soluble | C1D1 pre-dose, C1D1 post-dose, C2D1, C3D1, C5D1 | | | | Obtained prior to dosing. |
| Factors and miRNA | | | | | |
| Analyses | | | | | |
| Myeloid Derived | X | | | | Obtained prior to dosing. |
| Suppressor Cells | | | | | |
| (MDSCs) | | | | | |
| Peripheral Blood | C1D1 pre-dose, C1D1 post-dose, C2D1, C3D1, C5D1 | | | | Collected in USA and Canada Only. |
| Mononuclear Cells | | | | | Obtained prior to dosing. |
| (PBMCs) | | | | | |
| Optional Tumor | X | | | | As feasible obtained at baseline or any time on treatment |
| Biopsy Gene | | | | | |
| Expression Profiling | | | | | |
| Patient Reported | X | X | | After 6 | For C1D1 - LCSS and EQ-5D assessments performed |
| Outcomes | | | | months | after randomization PRIOR to first dose (day −3 to +1). |
| Assessment (PRO) | | | | | For on-study visits: Assessments (LCSS and EQ-5D) will |
| | | | | | be performed PRIOR to any study procedures and treatment. |
| | | | | | Assessments will be performed at each cycle on Day 1 |
| | | | | | for the first 6 months on study, then every 6 weeks |
| | | | | | thereafter for the remainder of the treatment period |
| Health Resource | | X | | | Except cycle 1. To include: concomitant medication |
| Utilization | | | | | collection |
| Clinical Drug | | | | | |
| Supplies | | | | | |
| IVRS Vial | X | X | | | Within 1 day prior to dosing |
| Assignment | | | | | |
| Platinum doublet | X | X | | | For Platinum doublet chemotherapy options & dose levels, |
| Chemotherapy | | | | | refer to, e.g., Table 16 and Table 17. Gemcitabine will |
| | | | | | be provided on day 1 and day 8 of each dosing cycle. |

| Maintenance Phase Dose Schedule: Optional maintenance | | |
|---|---|---|
| Pemetrexed for nonsquamous histology subjects only | | |
| Pemetrexed 500 mg/m2 | Week 1 Day 1 and Week 4 Day 1 | See, e.g., Table 17. |

^{a}If a dose is delayed, the procedures scheduled for that same time point should be delayed to coincide with when the time point's dosing actually occur

### Post-Treatment Follow-Up

The post-treatment follow-up begins when the decision to discontinue a subject from all treatment is made; this includes optional continuation maintenance therapy. Subjects who discontinue treatment for reasons other than disease progression will continue to have tumor assessments (if clinically feasible) according to the schedule in Table 23 until progression or the start of any subsequent therapy, whichever occurs first. Subjects are followed for drug-related toxicities until these toxicities resolve, return to baseline or are deemed irreversible. All adverse events are documented for a minimum of 100 days after the last dose of study medication. After completion of the first two follow-up visits, subjects are followed every 3 months for survival. Study assessments are to be collected as outlined in Table 23.

TABLE 23
Follow-up and Survival Procedures (CA209227) - All subjects

| | | | |
|---|---|---|---|
| | Follow- | Survival | |
| | Up^{a} | Follow-up | |
| Procedure | Visits 1 & | Visits^{b} | Notes |

SAFETY ASSESSMENTS

| | | | |
|---|---|---|---|
| Targeted Physical Examination | X | | To assess for potential late emergent study drug related issues. |
| Vital Signs | X | | |
| Adverse Event Assessment | X | X | |
| Review of Concomitant | X | | |
| Medications | | | |
| Laboratory Tests | X | | Required at Visit 1. Repeat at Visit 2 only if study drug related toxicity |
| | | | persists. |

EFFICACY ASSESSMENTS

| | | | |
|---|---|---|---|
| Radiographic Tumor | X | X | For subjects who discontinue study treatment for reasons |
| Assessment (CT/MRI of chest, | | | other than PD, follow up scans should be performed every 6 weeks (±1 wk) |
| abdomen, pelvis and known | | | up to first 12 months (week 48), then every 12 weeks until PD, lost to follow- |
| sites of disease) | | | up, or withdrawal of consent |
| | | | *Radiographic assessments for subjects who have not experienced PD must |
| | | | be obtained every 6 weeks (±7 days), and not delayed until follow-up visits 1 |
| | | | & 2. |
| Patient Reported Outcomes | X | EQ-5D | Both the LCSS and EQ-5D will be given in FU Visits 1 & 2. In Survival |
| Assessment(PRO) | | only | Visits, EQ-5D is collected every 3 months for the first year of the Follow-Up |
| | | | Phase, then every 6 months thereafter. |
| Pharmacokinetic & | X | | See, e.g., Tables 13-15 (For subjects treated in arms A, B, and C) |
| Immunogenicity | | | |
| Assessments | | | |
| Collection of Survival Status | X | X | Collect every 3 months in Survival Visits until death, lost to follow-up, or |
| and Subsequent Therapy | | | withdrawal of study consent. May be performed by phone contact or office visit. |

^{a}Follow-Up Visit 1 to occur 35 days from the last dose (±7 days) or coinciding with the date of discontinuation of study drug (±7 days) if the date of discontinuation is greater than 42 days from the last dose. Follow-Up Visit 2 to occur 80 days from Follow-Up Visit 1 (±7 days).
^{b}Survival Follow-Up Visits to occur approximately every 3 months from Follow-Up Visit 2.

### Sample Size

The sample size is calculated to compare OS between nivolumab and platinum doublet chemotherapy, and to compare OS between nivolumab in combination with ipilimumab and platinum doublet chemotherapy, at a Type I error level of 0.0167 (two-sided) and 90% power for each comparison. The number of events and power are calculated assuming an exponential distribution in platinum doublet chemotherapy arm and a piecewise mixture distribution in each of the experimental treatment arms.

Approximately 1200 subjects are randomized to the 4 treatment groups in a 1:1:1:1 ratio. The final analysis is conducted after 257 events occur in the control group, and these events will be monitored by the un-blinded independent statistician supporting the DMC. Assuming a 20% screening failure rate, it is estimated that approximately 1500 subjects will be enrolled in order to have 1200 subjects randomized, assuming a piecewise constant accrual rate (8 subjects/month during Months 1 to 2, 40 subjects/month during Months 3 to 4, 85 subjects/month during Months 5 to 6, 138 subjects/month during Months 7 to 8, 170 subjects/month after Month 8), it will take approximately 48 months to obtain the required number of death for the final OS analysis (14 months for accrual and 34 months for survival follow up).

### End Point

OS is a primary endpoint for this study. If OS superiority is demonstrated for at least one comparison, a gate keeping testing approach for the key secondary endpoints will be applied to additional experimental vs. control comparisons as described in the statistical analysis plan. Key secondary endpoints include PFS and ORR based on BICR assessments.

Each of the three primary OS analyses will be conducted using a two-sided log-rank test stratified by histology and PD-L1 status in all randomized subjects using Hochberg's procedure to address multiplicity. Hazard ratios (HR) and corresponding two-sided (1-adjusted α) % confidence intervals (CI) will be estimated using a Cox proportional hazard model, with treatment group as a single covariate, stratified by the above factors. OS curves, OS medians with 95% CIs, and OS rates at 12 and 24 months with 95% CIs will be estimated using Kaplan-Meier methodology. If OS superiority is demonstrated for at least one comparison, a gatekeeping testing approach for the key secondary endpoints will be applied to additional experimental vs. control comparisons as described in the statistical analysis plan. The key secondary endpoints will be tested in the following hierarchical order:

1) PFS (based on BICR assessments) analyses will be conducted using a two-sided log-rank test stratified by histology and PD-L1 status in all randomized subjects to compare each of the three experimental treatments to the control group. HRs and corresponding two-sided (1-adjusted α) % CIs will be estimated using a Cox proportional hazard model, with treatment group as a single covariate, stratified by the above factors. PFS curves, PFS medians with 95% CIs, and PFS rates at 6 and 12 months with 95% CIs will be estimated using Kaplan-Meier methodology.

2) ORR (based on BICR assessments) analyses will be conducted using a two-sided Cochran-Mantel-Haenszel (CMH) test stratified by PD-L1 status and histology to compare each of the three experiment treatments to the control group. Associated odds ratios and (1-adjusted α) % CI will also be calculated. Additionally, ORRs and their corresponding 95% exact CIs will be calculated using the Clopper-Pearson method for each of the four treatment groups.

3) Pairwise comparison of OS among experimental arms will be conducted using a two-sided log-rank test stratified by histology and PD-L1 status. HRs and corresponding two-sided (1-adjusted α) % CIs will be estimated using a Cox proportional hazard model, with treatment group as a single covariate, stratified by the above factors.

Descriptive analyses of PFS and ORR will be performed to evaluate differences between nivolumab monotherapy and nivolumab in combination of ipilimumab groups. These include HRs and medians with corresponding two-sided 95% CIs for PFS, as well as an ORR odds ratio with corresponding 95% CI.

### Analyses

Analyses of PD-L1 expression will be descriptive. Distribution of PD-L1 expression will be examined based on overall population. Potential associations between PD-L1 expression and efficacy measures (ORR, OS and PFS) will be assessed. If there is an indication of a meaningful association, further evaluation will be conducted to explore PD-L1 expression as a predictive biomarker by estimating the interaction effect between PD-L1 expression and treatment.

The results will show whether in chemotherapy-naive subjects with stage IV or recurrent NSCLC, the administration of nivolumab or nivolumab in combination with ipilimumab will improve overall survival (OS) compared with platinum-doublet chemotherapy.

### Example 2

A Phase 3b/4 Dose Frequency Optimization Study of Nivolumab 240 mg Every 2 Weeks vs. Nivolumab 480 mg Every 4 Weeks in Subjects with Advanced or Metastatic Non-small Cell Lung Cancer who Received 4 Months of Nivolumab at 3 mg/kg or 240 mg Every 2 Weeks

### Objectives

The coprimary objectives of this study are to compare PFS rate at 6 months after randomization and PFS rate at 1 year after randomization, as measured by investigator-assessed response using Response Evaluation Criteria in Solid Tumor (RECIST) 1.1 criteria, of nivolumab 240 mg every 2 weeks (Arm 1) and nivolumab 480 mg every 4 weeks (Arm 2) in subjects with advanced/metastatic (Stage IIIb/IV) NSCLC (non-Sq and Sq).

The secondary objectives of this study are: 1) to compare PFS rate in Arms 1 and 2 at 1 year after randomization by tumor histology and by response before randomization; 2) to compare PFS rate at 2 years after randomization in Arms 1 and 2; 3) To compare the overall survival (OS) rate at 1 year after randomization and up to 5 years after randomization in Arms 1 and 2, in all treated subjects, by tumor histology, and by response criteria before randomization; and 4) to assess safety and tolerability of nivolumab, as measured by the incidence and severity of adverse events (AEs) and specific laboratory abnormalities, in all treated subjects, in Arms 1 and 2, by tumor histology and response before randomization.

The exploratory objectives of this study are: 1) to characterize the pharmacokinetics of nivolumab at 240 mg and 480 mg and to explore relationship with respect to selected safety and efficacy endpoints; and 2) to assess health-related quality of life using the EQ-5D-3L.

### Investigational Plan

### Study Design and Duration

This is an open-label, randomized, Phase 3b/4 study comparing the efficacy of nivolumab 480 mg every 4 weeks vs. 240 mg every 2 weeks in adult patients with advanced/metastatic (Stage IIIb/Iv) NSCLC (non-Sq and Sq). Approximately 620 patients will be randomized 1:1 into 2 different dose regimens of nivolumab for a maximum of 5 years. Randomization will be stratified by histology and response criteria to pre-study nivolumab at randomization (CR or PR vs. SD). For subjects receiving nivolumab 240 mg every 2 weeks, each 14-day dosing period will constitute a cycle. For subjects receiving nivolumab 480 mg every 4 weeks, each 28-day dosing period will constitute a cycle. Investigational product will be provided at randomization. Subjects will continue treatment until disease progression or unacceptable toxicity for a maximum of 5 years from their first randomized dose. The follow-up period begins when the decision to permanently discontinue a subject from study therapy is made (no further treatment or retreatment with nivolumab is anticipated).

All patients will have received nivolumab (3 mg/kg or 240 mg) every 2 weeks for approximately 4 months (16 weeks ±2 weeks) and achieved a complete response (CR), partial response (PR), or stable disease (SD) to the nivolumab treatment as evidenced by a second tumor assessment prior to enrollment.

After this pre-study period, subjects will be enrolled, screened, and randomized 1:1 to receive either 240 mg every 2 weeks (Arm 1) or 480 mg every 4 weeks (Arm 2). Randomization will be stratified by histology and response criteria to pre-study nivolumab at randomization (CR or PR vs. SD). For subjects receiving nivolumab 240 mg every 2 weeks, each 14-day dosing period will constitute a cycle. For subjects receiving nivolumab 480 mg every 4 weeks, each 28-day dosing period will constitute a cycle. Investigational product will be provided at randomization.

Subjects are expected to have already completed their initial and second tumor assessments prior to enrollment. Once enrolled in this study, tumor assessments will continue every 8 weeks, which is similar to the standard of care assessment in this population.

Subjects will continue treatment until disease progression or unacceptable toxicity for a maximum of 5 years from their first randomized dose. The follow-up period begins when the decision to permanently discontinue a subject from study therapy is made (no further treatment or retreatment with nivolumab is anticipated) and will continue as specified.

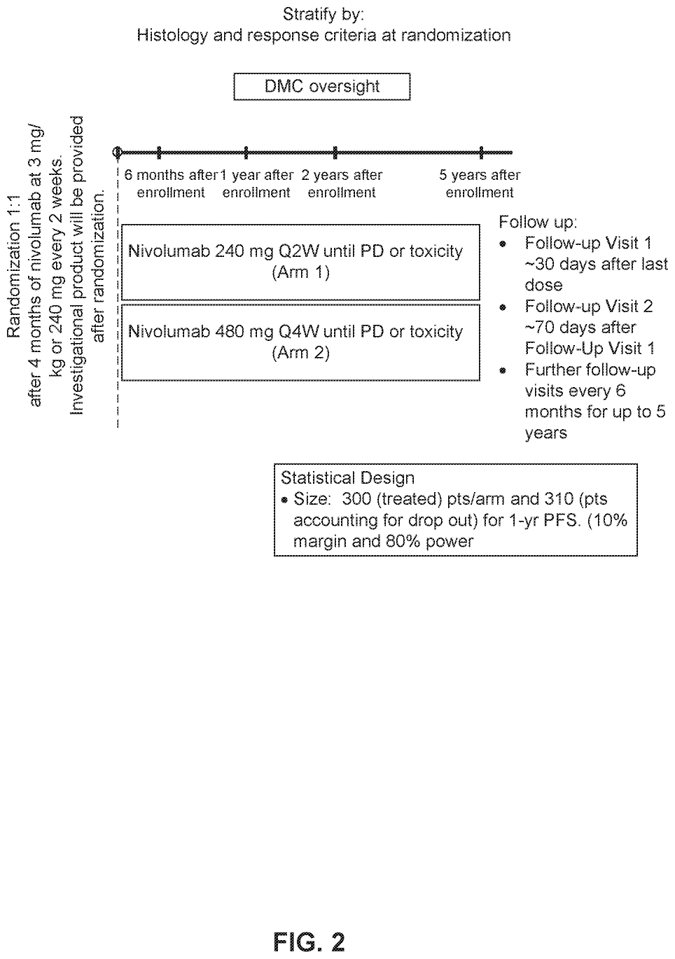

The study design schematic is presented in FIG. 2.

Each subject's last study visit will be defined as the last on-treatment or follow-up visit that occurs prior to the date of 5 years after the initiation of randomized therapy. The study will be completed no later than 5 years after the last subject's first visit.

### Post Study Access to Therapy

At the conclusion of the study, subjects who continue to demonstrate clinical benefit will be eligible to receive BMS-supplied study drug. Study drug will be provided via an extension of the study, a rollover study requiring approval by responsible health authority and ethics committee or through another mechanism at the discretion of BMS. BMS reserves the right to terminate access to BMS-supplied study drug if any of the following occur: a) the marketing application is rejected by responsible health authority; b) the study is terminated due to safety concerns; c) the subject can obtain medication from a government-sponsored or private health program; or d) therapeutic alternatives become available in the local market.

### Study Population

### Key Inclusion Criteria

For entry into the study, the following criteria MUST be met.

1) Signed Written Informed Consent
a) Subjects must have signed and dated an IRB/IEC-approved written informed consent form in accordance with regulatory and institutional guidelines prior to the performance of any protocol-related procedures that are not part of normal subject care. If a subject is not capable of giving informed consent, a legally acceptable representative may do so. However, if the subject subsequently becomes capable of making and communicating his or her informed consent during the study, consent must additionally be obtained from the subject.
b) Subjects must be willing and able to comply with scheduled visits, treatment schedule, laboratory tests, and other requirements of the study.

2) Target Population
a) Subjects with histologically or cytologically documented Sq- or non-SqNSCLC with Stage IIIB/Stage IV disease (according to version 7 of the International Association for the Study of Lung Cancer Staging Manual in Thoracic Oncology), or with recurrent or progressive disease following multimodal therapy (radiation therapy, surgical resection, or definitive chemoradiotherapy for locally advanced disease).
b) Subjects must have received and tolerated nivolumab 3 mg/kg or 240 mg every 2 weeks for approximately 4 months (16 weeks ±2 weeks). Subjects may continue to receive pre-study nivolumab treatment during screening assessments.
c) Subjects must have least 2 tumor assessments after the start of nivolumab and must demonstrate CR, PR, or SD to the pre-study nivolumab treatment on the latest scan within 28 days prior to randomization.
d) Subjects must have had measurable disease by CT or MRI per RECIST 1.1 criteria at the time of starting first dose of pre-study nivolumab treatment.
e) Subjects with a known activating epidermal growth factor receptors (EGFR) mutation or anaplastic lymphoma kinase (ALK) translocation must receive an EGFR or ALK TKI in addition to a platinum-based chemotherapy.
f) Eastern Cooperative Oncology Group (ECOG) Performance Status of 0-2.
g) Subjects with stable CNS metastases if CNS metastases are treated and subjects are neurologically returned to baseline (except for residual signs or symptoms related to the CNS treatment) for at least 2 weeks prior to enrollment. In addition, subjects must be either off corticosteroids or on a stable or decreasing dose of ≤10 mg daily prednisone (or equivalent).
h) All baseline laboratory requirements will be assessed and should be obtained within 14 days (unless otherwise specified). Screening laboratory values must meet the following criteria:
   i) WBCs ≥2000/μL
   ii) Neutrophils ≥1500/μL
   iii) Platelets ≥100×10³/μL
   iv) Hemoglobin ≥9.0 g/dL
   v) Serum creatinine of ≤1.5×ULN or creatinine clearance >40 mL/minute (using Cockcroft/Gault formula)

Female CrCl=[(140-age in years)×weight in kg X0.85]÷72×serum creatinine in mg/dL

Male CrCl=[(140-age in years)×weight in kg X1.00]÷72×serum creatinine in mg/dL
vi) AST ≤3×ULN
vii) ALT ≤3×ULN
viii) Total bilirubin ≤1.5×ULN (except subjects with Gilbert Syndrome who must have total bilirubin <3.0 mg/dL)
i) Palliative radiotherapy must be completed at least 2 weeks prior to enrollment.
j) Subject Re-enrollment: This study permits the re-enrollment of a subject that has discontinued the study as a pre-treatment failure (i.e., subject has not been randomized/has not been treated). If re-enrolled, the subject must be re-consented.

3) Age and Reproductive Status
a) Males and Females, ≥18 years of age.
b) Women of childbearing potential (WOCBP) must have a negative serum or urine pregnancy test (minimum sensitivity 25 IU/L or equivalent units of HCG) within 24 hours prior to the start of study drug.
c) Women must not be breastfeeding.
d) WOCBP must agree to follow instructions for method(s) of contraception for the duration of treatment with nivolumab plus 5 half-lives of nivolumab (125 days) plus 30 days (duration of ovulatory cycle) for a total of 155 days or 23 weeks post-treatment completion.
e) Males who are sexually active with WOCBP must agree to follow instructions for method(s) of contraception for the duration of treatment with nivolumab plus 5 half-lives of nivolumab (125 days) plus 90 days (duration of sperm turnover) for a total of 31 weeks post-treatment completion. In addition, male subjects must be willing to refrain from sperm donation during this time.
f) Azoospermic males are exempt from contraceptive requirements. WOCBP who are continuously not heterosexually active are also exempt from contraceptive requirements, and still must undergo pregnancy testing as described in this section.
g) At a minimum, subjects must agree to use 1 highly effective method of contraception.

Key Exclusion Criteria

For entry into the study, the following criteria MUST NOT be met.

1) Target Disease Exceptions
a) Subjects with carcinomatous meningitis.
b) Subjects with untreated, symptomatic central nervous system (CNS) metastases.

2) Medical History and Concurrent Diseases
a) Subjects with interstitial lung disease (e.g., sarcoidosis) that is symptomatic or may interfere with the detection or management of suspected drug-related pulmonary toxicity. Subjects with chronic obstructive pulmonary disease whose disease is controlled at study entry are allowed.
b) Subjects with an active, known or suspected autoimmune disease. Subjects with Type I diabetes mellitus, hypothyroidism only requiring hormone replacement, skin disorders (such as vitiligo, psoriasis, or alopecia) not requiring systemic treatment, or conditions not expected to recur in the absence of an external trigger are permitted to enroll.
c) Subjects with a condition requiring systemic treatment with either corticosteroids (>10 mg daily prednisone equivalent) or other immunosuppressive medications within 14 days of first randomized dose of study drug with the exception of the subjects allowed to enroll with treated or active CNS metastases requiring steroids. Inhaled or topical steroids, and adrenal replacement steroid doses >10 mg daily prednisone equivalent, are permitted in the absence of active autoimmune disease.
d) Subjects who received prior therapy with an anti-CTLA-4, anti-PD-L1, or anti-PD-L2, anti-CT137 (or any other antibody or drug specifically targeting T-cell costimulation or checkpoint pathways, except pre-study nivolumab) or subject is expected to require any other form of systemic antineoplastic therapy while receiving nivolumab.
e) Any other serious or uncontrolled medical disorder, active infection, physical exam finding, laboratory finding, altered mental status, or psychiatric condition that, in the opinion of the investigator, would limit the subject's ability to comply with the study requirements, substantially increase the risk to the subject, or impact the interpretability of study results.
f) Other active malignancy requiring concurrent intervention.
g) Subjects with previous malignancies (except non-melanoma skin cancers, and the following in situ cancers: bladder, gastric, colon, endometrial, cervical/dysplasia, melanoma, or breast) are excluded unless a complete remission was achieved at least 2 years prior to study entry AND no additional therapy is required during the study period with the exception of anti-estrogen/androgen therapy or bisphosphonates.
h) All toxicities attributed to prior anti-cancer therapy other than alopecia, fatigue, or peripheral neuropathy must have resolved to Grade 1 (NCI CTCAE version 4) or baseline before administration of study drug.
i) Subjects must have recovered from the effects of major surgery or significant traumatic injury at least 14 days before the first dose of study treatment.

3) Physical and Laboratory Test Findings
a) Positive for Hepatitis B virus or Hepatitis C virus indicating acute or chronic infection.
b) Positive HIV test or Acquired Immunodeficiency Syndrome (AIDS)

4) Allergies and Adverse Drug Reaction
a) History of severe hypersensitivity reactions to other monoclonal antibodies.

5) Other Exclusion Criteria
a) Prisoners or subjects who are involuntarily incarcerated. (Note: under certain specific circumstances a person who has been imprisoned may be included or permitted to continue as a subject. Strict conditions apply, and Bristol-Myers Squibb approval is required.)
b) Subjects who are compulsorily detained for treatment of either a psychiatric or physical (e.g., infectious disease) illness.

### Concomitant Treatments

Prohibited and/or Restricted Treatments

The following medications are prohibited during the study (unless utilized to treat a drug related AE): 1) immunosuppressive agents; 2) immunosuppressive doses of systemic corticosteroids (exception: topical, ocular, intra-articular, intranasal, and inhalational corticosteroids with minimal systemic absorption allowed); and 3) any concurrent anti-neoplastic therapy (i.e., chemotherapy, hormonal therapy, immunotherapy, extensive, non-palliative radiation therapy, or standard or investigational agents for treatment of NSCLC).

### Other Restrictions and Precautions

Subjects with a condition requiring systemic treatment with either corticosteroids (>10 mg daily prednisone equivalent) or other immunosuppressive medications within 14 days of randomization are excluded. Inhaled or topical steroids, and adrenal replacement steroid doses >10 mg daily prednisone equivalent, are permitted in the absence of active autoimmune disease.

### Permitted Therapy

Subjects are permitted the use of topical, ocular, intra-articular, intranasal, and inhalational corticosteroids (with minimal systemic absorption). Adrenal replacement steroid doses >10 mg daily prednisone are permitted. A brief (less than 3 weeks) course of corticosteroids for prophylaxis (e.g., contrast dye allergy) or for treatment of non-autoimmune conditions (e.g., delayed-type hypersensitivity reaction caused by a contact allergen) is permitted.

Regular concomitant use of bisphosphonates and RANK-L inhibitors for prevention or reduction of skeletal-related events in patients with bone metastases is allowed if initiated prior to first dose of study therapy. Prior palliative radiotherapy must have been completed at least 2 weeks prior to randomization.

Discontinuation of Subjects Following any Treatments with Study Drug

Subjects MUST discontinue investigational product (and non-investigational product at the discretion of the investigator) for any of the following reasons:
1) Subject's request to stop study treatment.
2) Any clinical adverse event (AE), laboratory abnormality or intercurrent illness which, in the opinion of the investigator, indicates that continued participation in the study is not in the best interest of the subject.
3) Termination of the study by Bristol-Myers Squibb (BMS).
4) Loss of ability to freely provide consent through imprisonment or involuntarily incarceration for treatment of either a psychiatric or physical (e.g., infectious disease) illness.
5) Criteria described under “Dose Discontinuation Criteria.”
6) Subject is a normal healthy female and becomes pregnant. Within 24 hours of awareness, investigator must notify the BMS Medical Monitor/designee of the pregnancy. In most cases, the study drug will be permanently discontinued in an appropriate manner (e.g., dose tapering if necessary for subject safety). If the investigator determines a possible favorable benefit/risk ratio that warrants continuation of study drug, a discussion between the investigator and the BMS Medical Monitor/designee must occur.

All subjects who discontinue study drug should comply with protocol specified follow-up procedures. The only exception to this requirement is when a subject withdraws consent for all study procedures including post-treatment study follow-up or loses the ability to consent freely (i.e., is imprisoned or involuntarily incarcerated for the treatment of either a psychiatric or physical illness). If study drug is discontinued prior to the subject's completion of the study, the reason for the discontinuation must be documented in the subject's medical records and entered on the appropriate case report form (CRF) page.

### Post Study Drug Study Follow Up

In this study, PFS is a key endpoint of the study. Post-study follow-up is of critical importance and is essential to preserving subject safety and the integrity of the study. Subjects who discontinue study drug must continue to be followed for collection of outcome and/or survival follow-up data as required until death or the conclusion of the study.

### Withdrawal of Consent

Subjects who request to discontinue study drug will remain in the study and must continue to be followed for protocol specified follow-up procedures. The only exception to this is when a subject specifically withdraws consent for any further contact with him/her or persons previously authorized by subject to provide this information. Subjects should notify the investigator of the decision to withdraw consent from future follow-up in writing, whenever possible. The withdrawal of consent should be explained in detail in the medical records by the investigator, as to whether the withdrawal is from further treatment with study drug only or also from study procedures and/or post treatment study follow-up, and entered on the appropriate CRF page. In the event that vital status (whether the subject is alive or dead) is being measured, publicly available information should be used to determine vital status only as appropriately directed in accordance with local law.

### Lost to Follow-Up

All reasonable efforts must be made to locate subjects to determine and report their ongoing status. This includes follow-up with persons authorized by the subject as noted above. Lost to follow-up is defined by the inability to reach the subject after a minimum of three documented phone calls, faxes, or emails as well as lack of response by subject to one registered mail letter. All attempts should be documented in the subject's medical records. If it is determined that the subject has died, the site will use permissible local methods to obtain the date and cause of death.

If investigator's use of third-party representative to assist in the follow-up portion of the study has been included in the subject's informed consent, then the investigator may use a Sponsor-retained third-party representative to assist site staff with obtaining subject's contact information or other public vital status data necessary to complete the follow-up portion of the study. The site staff and representative will consult publicly available sources, such as public health registries and databases, in order to obtain updated contact information. If after all attempts, the subject remains lost to follow-up, then the last known alive date as determined by the investigator should be reported and documented in the subject's medical records.

### Study Drug

Study drug includes both Investigational [Medicinal] Product (IP/IMP) and Non-investigational [Medicinal] Product (Non-IP/Non-IMP). An investigational product, also known as investigational medicinal product in some regions, is defined a pharmaceutical form of an active substance or placebo being tested or used as a reference in a clinical study, including products already with a marketing authorization but used or assembled (formulated or packaged) differently than the authorized form, or used for an unauthorized indication, or when used to gain further information about the authorized form. Other medications used as support or escape medication for preventative, diagnostic, or therapeutic reasons, as components of the standard of care for a given diagnosis, may be considered as non-investigational products (not applicable).

### Method of Assigning Subject Identification

This is a randomized study. After the subject's eligibility is established and informed consent has been obtained, the subject will be enrolled, and a number will be assigned through an interactive web-based response system (IWRS). Specific instructions for enrollment and randomization procedures using IWRS will be provided to the investigational site in a separate document/manual. Subjects meeting all eligibility criteria and randomized into the study will be assigned to 1 of the 2 treatment arms and stratified by the following factors: histology and response at randomization (CR or PR or SD).

### Selection and Timing of Dose for Each Subject

Subjects will enroll after receiving approximately 4 months (16 weeks ±2 weeks) of nivolumab therapy and after receiving a second tumor assessment with evidence of a CR, PR, or SD.

Subjects in Arm 1 will receive 240 mg of nivolumab intravenously as a 30 minute (±5 minutes) IV infusion on Day 1 of each treatment cycle every 2 weeks, until progression, unacceptable toxicity, withdrawal of consent, or the subject reaches a maximum of 5 years from the first on-study dose, or the study ends, whichever occurs first. In this arm, each 14-day dosing period will constitute a cycle.

Subjects in Arm 2 will receive 480 mg nivolumab as a 30 minute (±5 minutes) IV infusion on Day 1 of each treatment cycle every 4 weeks until progression, unacceptable toxicity, withdrawal of consent, or the subject reaches a maximum of 5 years from the first on-study dose, whichever occurs first. In this arm, each 28-day dosing period will constitute a cycle.

Subjects in Arm 1 may be dosed no less than 12 days from the previous dose; subjects in Arm 2 may be dosed no less than 26 days from the previous dose.

No dose escalations or reductions of nivolumab are allowed. There are no pre-medications recommended for nivolumab until infusion reactions have been observed in the subject. Subjects should be carefully monitored for infusion reactions during nivolumab administration.

### Dose Delay Criteria

Nivolumab administration should be delayed for the following:
1) Any Grade ≥2 drug-related AE, with the following exceptions:
   a) Grade 2 drug-related skin AEs, fatigue or laboratory abnormalities—no treatment delay required.
2) Any Grade 3 skin, drug-related AE.
3) Any Grade 3 drug-related laboratory abnormality, with the following exceptions for lymphopenia, AST, ALT, or total bilirubin or asymptomatic amylase or lipase:
   a) Grade 3 lymphopenia does not require dose delay
   b) If a subject has a baseline AST, ALT, or total bilirubin that is within normal limits, delay dosing for drug-related Grade ≥2 toxicity
   c) If a subject has baseline AST, ALT, or total bilirubin within the Grade 1 toxicity range, delay dosing for drug-related Grade ≥3 toxicity
   d) Any Grade ≥3 drug-related amylase or lipase abnormality that is not associated with symptoms or clinical manifestations of pancreatitis does not require dose delay. The Medical Monitor should be consulted for such Grade ≥3 amylase or lipase abnormalities.
4) Any AE, laboratory abnormality, or intercurrent illness which, in the judgment of the investigator, warrants delaying the dose of study medication.

Subjects who require delay of nivolumab should be re-evaluated weekly or more frequently if clinically indicated and resume nivolumab dosing when re-treatment criteria are met.

### Criteria to Resume Treatment

Subjects may resume treatment with nivolumab when the drug-related AE(s) resolve(s) to Grade ≤1 or baseline, with the following exceptions:
1) Subjects may resume treatment in the presence of Grade 2 fatigue.
2) Subjects who have not experienced a Grade 3 drug-related skin AE may resume treatment in the presence of Grade 2 skin toxicity.
3) Subjects with baseline Grade 1 AST/ALT or total bilirubin who require dose interruption for reasons other than a 2-grade shift in AST/ALT or total bilirubin may resume treatment in the presence of Grade 2 AST/ALT OR total bilirubin value.
4) Subjects with combined Grade 2 AST/ALT AND total bilirubin values meeting discontinuation parameters (under “Dose Discontinuation Criteria” provided below) should have treatment permanently discontinued.
5) Drug-related pulmonary toxicity, diarrhea, or colitis must have resolved to baseline before treatment is resumed. Subjects with persistent Grade 1 pneumonitis after completion of a steroid taper over at least 1 month may be eligible for retreatment if discussed with and approved by the Medical Monitor.
6) Drug-related endocrinopathies adequately controlled with only physiologic hormone replacement may resume treatment after consultation with the Medical Monitor.

Dose interruption of nivolumab which results in treatment interruption of >6 weeks require treatment discontinuation, with exceptions as noted below under “Dose Discontinuation Criteria.” There will be no dose reductions for nivolumab.

### Dose Discontinuation Criteria

Nivolumab treatment should be permanently discontinued for the following:
1) Any Grade 2 drug-related uveitis, eye pain, or blurred vision that does not respond to topical therapy and does not improve to Grade 1 severity within the re-treatment period OR requires systemic treatment.
2) Any Grade 3 non-skin, drug-related AE lasting >7 days, with the following exceptions for laboratory abnormalities, drug-related uveitis, pneumonitis, bronchospasm, hypersensitivity reactions, infusion reactions, and endocrinopathies:
   a) Grade 3 drug-related uveitis, pneumonitis, bronchospasm, hypersensitivity reaction, or infusion reaction of any duration requires discontinuation.
   b) Grade 3 drug-related endocrinopathies adequately controlled with only physiologic hormone replacement do not require discontinuation.
   c) Grade 3 drug-related laboratory abnormalities do not require treatment discontinuation except:
      i) Grade 3 drug-related thrombocytopenia >7 days or associated with bleeding requires discontinuation.
3) Any drug-related liver function test (LFT) abnormality that meets the following criteria require discontinuation:
   a) AST or ALT >5-10×ULN for >2 weeks
   b) AST or ALT >10×ULN
   c) Total bilirubin >5×ULN
   d) Concurrent AST or ALT >3×ULN and total bilirubin >2×ULN.
4) Any Grade 4 drug-related AE or laboratory abnormality, except for the following events which do not require discontinuation:
   a) Grade 4 neutropenia ≤7 days
   b) Grade 4 lymphopenia or leukopenia
   c) Isolated Grade 4 amylase or lipase abnormalities that are not associated with symptoms or clinical manifestations of pancreatitis. The Medical Monitor should be consulted for Grade 4 amylase or lipase abnormalities.
   d) Isolated Grade 4 electrolyte imbalances/abnormalities that are not associated with clinical sequelae and are corrected with supplementation/appropriate management within 72 hours of their onset
   e) Grade 4 drug-related endocrinopathy AE, such as adrenal insufficiency, ACTH deficiency, hyper- or hypothyroidism, or glucose intolerance, which resolve or are adequately controlled with physiologic hormone replacement (corticosteroids, thyroid hormones) or glucose-controlling agents, respectively, may not require discontinuation after discussion with and approval from the Medical Monitor.
5) Any event that leads to interruption in dosing lasting >6 weeks from the previous dose requires discontinuation, with the following exceptions:
   a) Dosing interruptions to allow for prolonged steroid tapers to manage drug-related adverse events are allowed. Prior to re-initiating treatment in a subject with a dosing interruptions lasting >6 weeks from the previous dose, the Medical Monitor must be consulted. Tumor assessments should continue as per protocol even if dosing is interrupted. Periodic study visits to assess safety and laboratory studies should also continue every 6 weeks or more frequently if clinically indicated during such dosing interruptions.
   b) Dosing interruptions lasting >6 weeks from the previous dose that occur for non-drug related reasons may be allowed if approved by the Medical Monitor. Prior to re-initiating treatment in a subject with a dosing interruption lasting >6 weeks, the Medical Monitor must be consulted. Tumor assessments should continue as per protocol even if dosing is interrupted. Periodic study visits to assess safety and laboratory studies should also continue every 6 weeks or more frequently if clinically indicated during such dosing interrupted.
6) Any AE, laboratory abnormality, or intercurrent illness which, in the judgment of the Investigator, presents a substantial clinical risk to the subject with continued nivolumab dosing.

### Treatment of Nivolumab-Related Infusion Reactions

Since nivolumab contains only human immunoglobulin protein sequences, it is unlikely to be immunogenic and induce infusion or hypersensitivity reactions. However, if such a reaction were to occur, it might manifest with fever, chills, rigors, headache, rash, pruritus, arthralgias, hypotension, hypertension, bronchospasm, or other allergic-like reactions. All Grades 3 or 4 infusion reactions should be reported within 24 hours to the study medical monitor and reported as an SAE if it meets the criteria. Infusion reactions should be graded according to NCI CTCAE (Version 4.0) guidelines.

Treatment recommendations are provided below and may be modified based on local treatment standards and guidelines, as appropriate:

For Grade 1 symptoms: (mild reaction; infusion interruption not indicated; intervention not indicated):
1) Remain at bedside and monitor subject until recovery from symptoms. The following prophylactic pre-medications are recommended for future infusions: diphenhydramine 50 mg (or equivalent) and/or acetaminophen/paracetamol 325 to 1000 mg at least 30 minutes before additional nivolumab administrations.

For Grade 2 symptoms: (moderate reaction required therapy or infusion interruption but responds promptly to symptomatic treatment (e.g., antihistamines, non-steroidal anti-inflammatory drugs, narcotics, corticosteroids, bronchodilators, IV fluids); prophylactic medications indicated for ≤24 hours):
1) Stop the nivolumab infusion, begin an IV infusion of normal saline, and treat the subject with diphenhydramine 50 mg IV (or equivalent) and/or acetaminophen/paracetamol 325 to 1000 mg; remain at bedside and monitor subject until resolution of symptoms. Corticosteroid and/or bronchodilator therapy may also be administered as appropriate. If the infusion is interrupted, then restart the infusion at 50% of the original infusion rate when symptoms resolve; if no further complications ensue after 30 minutes, the rate may be increased to 100% of the original infusion rate. Monitor subject closely. If symptoms recur, then no further BMS-936558 will be administered at that visit.
2) For future infusions, the following prophylactic pre-medications are recommended: diphenhydramine 50 mg (or equivalent) and/or acetaminophen/paracetamol 325 to 1000 mg should be administered at least 30 minutes before nivolumab infusions. If necessary, corticosteroids (up to 25 mg of SoluCortef or equivalent) may be used.

For Grade 3 or 4 symptoms: (severe reaction, Grade 3: prolonged [i.e., not rapidly responsive to symptomatic medication and/or brief interruption of infusion]; recurrence of symptoms following initial improvement; hospitalization indicated for other clinical sequelae (e.g., renal impairment, pulmonary infiltrates). Grade 4: Life-threatening; pressor or ventilatory support indicated):
1) Immediately discontinue infusion of nivolumab. Begin an IV infusion of normal saline and treat the subject as follows: Recommend bronchodilators, epinephrine 0.2 to 1 mg of a 1:1000 solution for subcutaneous administration or 0.1 to 0.25 mg of a 1:10,000 solution injected slowly for IV administration, and/or diphenhydramine 50 mg IV with methylprednisolone 100 mg IV (or equivalent), as needed. Subject should be monitored until the Investigator is comfortable that the symptoms will not recur. Nivolumab will be permanently discontinued. Investigators should follow their institutional guidelines for the treatment of anaphylaxis. Remain at bedside and monitor subject until recovery of the symptoms.

In case of late-occurring hypersensitivity symptoms (e.g., appearance of a localized or generalized pruritus within 1 week after treatment), symptomatic treatment may be given (e.g., oral antihistamine or corticosteroids).

### Treatment Compliance

Treatment compliance will be monitored by drug accountability as well as the subject's medical record and eCRF.

### Study Assessments and Procedures

### Safety Assessments

Safety assessments will be conducted throughout the trial and during 100 days after the last dose of study treatment. The assessments should be monitored starting on Cycle 1 Day 1 until discontinuation from study therapy (unless otherwise noted).

Additional procedures and assessments may be performed as part of standard of care; however, data for these assessments should remain in the patient's medical record and should not be provided to BMS unless specifically requested. NCI CTCAE version 4.0 will be the criteria used to assess severity of AEs.

To assure an ongoing favorable risk/benefit assessment for subjects enrolled in this study, an independent Data Monitoring Committee (DMC) will be used to monitor the safety and activity of the treatments throughout the conduct of the trial.

### Efficacy Assessments

Efficacy assessments will take place according to Tables 24 and should be performed, starting with Cycle 1 Day 1, according to RECIST 1.1 criteria.

High resolution CT with oral or IV contrast or contrast-enhanced MM are the preferred imaging modalities for assessing radiographic tumor response. If a subject has a known allergy to contrast material, please use local prophylaxis standards to obtain the assessment with contrast if at all possible or use an alternate modality. In cases where contrast is strictly contraindicated, a non-contrast scan will suffice. Screening assessments, including chest, abdomen, pelvis, brain, and all known or suspected sites of disease, should be performed within 28 days of first dose of study drug. Brain Mill is the preferred imaging method when evaluating CNS metastasis is necessary. In addition to chest and abdomen, all known or suspected sites of disease (including CNS) should be assessed at subsequent assessments using the same imaging method and technique. If more than one method is used at screening, then the most accurate method according to RECIST 1.1 should be used when recording data and should again be used for all subsequent assessments. Bone scan, PET scan, or ultrasound is not adequate for assessment of RECIST response. In selected circumstances where such modalities are the sole modality used to assess certain non-target organs, those non-target organs may be evaluated less frequently. For example, bone scans may need to be repeated only when complete response is identified in the target disease or when progression in bone is suspected. Previously treated CNS metastases are not considered measurable lesions for purposes of RECIST determined response.

Tumor measurements should be made by the same investigator or radiologist for each assessment whenever possible. Changes in tumor measurements and tumor responses to guide ongoing study treatment decisions should be assessed by the investigator using RECIST 1.1.

### Pharmacokinetic Assessments

Samples for pharmacokinetic (PK) assessments will be collected for all subjects receiving nivolumab. All time points are relative to the start of study drug administration. All on-treatment time points are intended to align with days on which study drug is administered; if dosing occurs on a different day, the PK sampling should be adjusted accordingly. PK samples will be analyzed for nivolumab by a validated immunoassay.

### Outcome Research Assessments

The EQ-5D-3L comprises 5 dimensions (mobility, self-care, usual activities, pain/discomfort, and anxiety) and a visual analog rating scale. The responses to the EQ-5D-3L domains will be converted to health status index based on the European scoring algorithm.

### Adverse Events

An Adverse Event (AE) is defined as any new untoward medical occurrence or worsening of a preexisting medical condition in a clinical investigation subject administered study drug and that does not necessarily have a causal relationship with this treatment. An AE can therefore be any unfavorable and unintended sign (such as an abnormal laboratory finding), symptom, or disease temporally associated with the use of study drug, whether or not considered related to the study drug.

The causal relationship to study drug is determined by a physician and should be used to assess all adverse events (AE). The causal relationship can be one of the following:

Related: There is a reasonable causal relationship between study drug administration and the AE.

Not related: There is not a reasonable causal relationship between study drug administration and the AE.

The term “reasonable causal relationship” means there is evidence to suggest a causal relationship.

Adverse events can be spontaneously reported or elicited during open-ended questioning, examination, or evaluation of a subject. (In order to prevent reporting bias, subjects should not be questioned regarding the specific occurrence of one or more AEs.)

BMS will be reporting adverse events to regulatory authorities and ethics committees according to local applicable laws including European Directive 2001/20/EC and FDA Code of Federal Regulations 21 CFR Parts 312 and 320.

Suspected Unexpected Serious Adverse Reaction is a serious adverse event that is both unexpected and related to an IMP or comparator IMP, for which expedited reporting to clinical investigators, Ethics Committees and Health Authorities is required (Previously known as ESR).

A non-serious adverse event is an AE not classified as serious.

### Serious Adverse Event Collection and Reporting

Following the subject's written consent to participate in the study, all serious adverse events (SAEs), whether related or not related to study drug, must be collected, including those thought to be associated with protocol-specified procedures. All SAEs must be collected that occur during the screening period and within 100 days of discontinuation of dosing.

The investigator must report any SAE that occurs after these time periods and that is believed to be related to study drug or protocol-specified procedure.

An SAE report must be completed for any event where doubt exists regarding its seriousness.

If the investigator believes that an SAE is not related to study drug, but is potentially related to the conditions of the study (such as withdrawal of previous therapy or a complication of a study procedure), the relationship must be specified in the narrative section of the SAE Report Form.

SAEs, whether related or not related to study drug, and pregnancies must be reported to BMS (or designee) within 24 hours of awareness of the event. SAEs must be recorded on the SAE Report Form. The preferred method for SAE data reporting collection is through the eCRF.

If an ongoing SAE changes in its intensity or relationship to study drug or if new information becomes available, the SAE report must be updated and submitted within 24 hours to BMS (or designee) using the same procedure used for transmitting the initial SAE report.

All SAEs must be followed to resolution or stabilization.

### Non-Serious Adverse Event Collection and Reporting

The collection of non-serious adverse event (AE) information should begin at initiation of study drug. Non-serious AE information should also be collected from the start of a placebo lead-in period or other observational period intended to establish a baseline status for the subjects.

Non-serious AEs should be followed to resolution or stabilization, or reported as SAEs if they become serious. Follow-up is also required for non-serious AEs that cause interruption or discontinuation of study drug and for those present at the end of study treatment as appropriate. All identified non-serious AEs must be recorded and described on the non-serious AE page of the CRF (paper or electronic).

### Statistical Considerations

### Sample Size Determination

The primary analyses evaluates the non-inferiority of post-randomization 6-months and 12-month milestone PFS rate of nivolumab 480 mg Q4W versus the PFS rate of nivolumab 240 mg Q2W in subjects with disease control (CR/PR/SD) after approximately 4 months (16 weeks ±2 weeks) of nivolumab 3 mg/kg or 240 mg Q2W treatment. The non-inferiority margin of −10% was chosen for this study. Patients who achieved CR, PR, or SD will be randomized after 4 months (16 weeks ±2 weeks) of nivolumab treatment. It is estimated that the 12-month milestone PFS rate post-randomization is 0.384 with 240 mg Q2W and 6-month PFS rate post-randomization is 0.52.

The sample size was computed based on a cumulative hazard function which accounts for both progression and censoring distributions. Using cumulative hazard function and its relation with survival function, it is estimated that 600 patients, 300 in each arm, will provide 80% power for the lower bound of a 95.3% one-sided confidence interval above −10% at the 12-month milestone and the lower bound of a 99.1% confidence interval above −10% at 6 months if PFS rates of the 2 arms are assumed to be equal. The experiment-wise error rate is maintained at one-sided 5% level.

To account for those who are randomized but not receiving treatment, 310 per arm will be randomized. With a 15% screen failure rate, approximately 730 subjects will be screened to achieve approximately 620 randomized subjects.

### Population for Analyses

All enrolled subjects: all subjects who signed an informed consent form and were registered into the IWRS.

All randomized subjects: all subjects who are randomized to 240 mg every 2 weeks or 480 mg every 4 weeks. This is the primary population for efficacy analyses. Subpopulation analyses will be conducted by tumor histology (Sq or non-Sq) and response at randomization (PR or CR vs SD).

All treated subjects: all randomized subjects who received at least 1 dose of nivolumab. This is the primary population for safety analyses. Subpopulation analyses will be conducted by tumor histology and response at randomization for some safety variables.

PK subjects: all treated subjects with available serum time-concentration data.

### Endpoints

### Primary Endpoint(s)

The coprimary objectives of this study will be assessed by PFS rate at 6 months after randomization and PFS rate at 1 year after randomization. PFS is defined as the time from the date of randomization to the date of first documented tumor progression determined by the investigator or death, whichever is earlier. Subjects who did not progress or die will be censored on the date of their last evaluable tumor assessment. The PFS rate at 6 months is the rate from Kaplan-Meier (KM) estimate 6 months after randomization; PFS rate at 1 year is the rate from KM estimate at 1 year after randomization.

### Secondary Endpoint(s)/Objectives

The secondary objectives of this study will be assessed by: 1) PFS rate at 1 year after randomization by tumor histology and by response criteria; 2) PFS rate at 2 years after randomization; 3) OS rate at 1 year and OS up to 5 years by arm, histology, and response status at randomization. OS is defined as time from the date of randomization to the date of death. Subjects who did not die by the end of the study will be censored at the last known date alive. OS rate at 1 year is the rate from KM estimated at 1 year after randomization; and 4) Safety and tolerability of nivolumab, as measured by incidence and severity of AEs and specific laboratory abnormalities.

### Exploratory Endpoint(s)

The exploratory objectives of this study will be assessed by: 1) the relationship of pharmacokinetics of nivolumab with respect to selected safety and efficacy endpoints at 240 mg Q2W and 480 mg Q4W; and 2) EQ-5D-3L.

### Analysis

### Demographics and Baseline Characteristics

Demographics and baseline disease characteristics including age, sex, race, ethnicity, weight, baseline disease diagnosis, and medical condition will be summarized using descriptive statistics by dose regimen.

### Efficacy Analysis

PFS will be summarized by KM product-limit method and confidence interval for hazard ratio will be produced from a stratified (by tumor histology and response category) proportional hazard model. Median values of PFS, along with one-sided 95% CI using the Brookmeyer and Crowley method, will be calculated. The status of subjects who are censored in the PFS KM analysis will be tabulated for each dose regimen.

The 95% one-sided confidence intervals for PFS rates at 6 and 12 months will be calculated using the Greenwood formula for each dose regimen and difference between dose regimens.

For the 6-month analysis, the 99.1% one-sided confidence interval around the difference in PFS rates between Q2W dose regimen and the Q4W regimen will be generated. If the lower bound of the confidence interval is above −10%, non-inferiority will be claimed.

A 95.3% unadjusted confidence interval will be used at 12 months. If the lower limit of the confidence interval (Q4W-Q2W) is above −10%, it is considered that the Arm 2 (480 mg Q4W) is non-inferior to Arm 1 (240 mg Q2W).

The OS and OS rates at 6 months and 12 months will be analyzed using the same method as for PFS and PFS rates.

### Safety Analysis

Safety will be analyzed through the incidence of deaths, AEs, SAES, AEs leading to discontinuation, AEs leading to dose interruption, select AEs, and specific laboratory abnormalities (worst grade) in each arm. Toxicities will be graded using the NCI CTCAE version 4.0.

### Pharmacokinetic Analysis

The nivolumab serum concentration data from this study may be combined with data from other nivolumab studies in the population pharmacokinetic model. These models may be used to evaluate the effects of intrinsic and extrinsic covariates on the pharmacokinetics of nivolumab and to determine measures of individual exposure. In addition, model determined exposures may be used for exposure-response analyses. Results of population pharmacokinetics and exposure-response analyses will be reported separately.

### Outcomes Research Analysis

The EQ-5D-3L will be used to assess the subject's overall health status. The EQ-SD-3L descriptive system comprises the following 5 dimensions: mobility, self-care, usual activities, pain/discomfort, and anxiety/depression. Each dimension has 3 levels: no problems, some problems, and severe problems. The EQ visual analog scale (VAS) records the subject's self-rated health state on a 100-point, vertical visual analogue scale (0=worst imaginable health state; 100=best imaginable health state).

Subject's overall health state on a visual analog scale (EQ-VAS) at each assessment time point as well as change from baseline will be summarized using descriptive statistics by arm (including mean and 95% confidence interval), as randomized.

Proportion of subjects reporting problems for the 5 EQ-5D-3L dimensions at each assessment time point will be summarized by level of problem and by arm, as randomized. Percentages will be assessed on number of subjects assessed at assessment time point.

Summary statistics will be calculated for the population preference-based health state utility score (EQ-5D-3L Index) at each assessment as well as patients' change from baseline at each assessment by treatment arm, as randomized.

1. A method for treating a subject afflicted with a lung cancer comprising administering the subject in need thereof a therapeutically effective amount of:
(a) an anti-cancer agent which is an antibody or an antigen-binding portion thereof that binds specifically to a Programmed Death-1 (PD-1) receptor and inhibits PD-1 activity (“an anti-PD-1 antibody or antigen-binding portion thereof”), which is administered by infusion for less than 60 minutes, in combination with,
(b) an antibody or antigen-binding portion thereof that binds specifically to Cytotoxic T-Lymphocyte Antigen-4 (CTLA-4) and inhibits CTLA-4 activity (“an anti-CTLA-4 antibody or antigen-binding portion thereof”) which is administered by infusion for less than 90 minutes.

2. The method of claim 1, wherein the anti-PD-1 antibody or antigen-binding portion thereof comprises nivolumab.

3. The method of claim 1, wherein the anti-PD-1 antibody or antigen-binding portion thereof comprises pembrolizumab.

4. (canceled)

5. The method of claim 1, wherein the anti-CTLA-4 antibody or antigen-binding portion thereof comprises ipilimumab.

6. The method of claim 1, wherein the anti-CTLA-4 antibody or antigen-binding portion thereof comprises tremelimumab.

7. The method of claim 1, wherein the anti-PD-1 antibody or antigen-binding portion thereof and the anti-CTLA-4 antibody or antigen-binding portion thereof are administered concurrently in separate compositions.

8. The method of claim 1, wherein the anti-PD-1 antibody or antigen-binding portion thereof is administered at a flat dose.

9. The method of claim 1, wherein the anti-PD-1 antibody or antigen-binding portion thereof is administered at a flat dose of at least about 240 mg.

10. The method of claim 1, wherein the anti-PD-1 antibody or antigen-binding portion thereof is administered at a flat dose of about 240 mg once about every 2 weeks.

11. A method for treating a subject afflicted with a lung cancer comprising administering to the subject in need thereof a flat dose of a therapeutically effective amount of an anti-cancer agent which is an antibody or an antigen-binding portion thereof that binds specifically to a Programmed Death-1 (PD-1) receptor and inhibits PD-1 activity (“an anti-PD-1 antibody or antigen-binding portion thereof”).

12. The method of claim 11, wherein the anti-PD-1 antibody or antigen-binding portion thereof is administered at a flat dose of at least about 240 mg.

13. The method of claim 11, wherein the anti-PD-1 antibody or antigen-binding portion thereof is administered at a flat dose of about 240 mg once about every 2 weeks.

14. A kit for treating a subject afflicted with a lung cancer, the kit comprising:
(a) a flat dosage of at least about 240 mg or about 480 mg of an antibody or an antigen-binding portion thereof that specifically binds to the PD-1 receptor and inhibits PD-1 activity (“an anti-PD-1 antibody or an antigen-binding portion thereof”); and
(b) instructions for using the anti-PD-1 antibody or antigen-binding portion thereof in the method of claim 8.

15. A kit for treating a subject afflicted with a lung cancer, the kit comprising:
(a) a flat dosage of about 240 mg or about 480 mg of an anti-cancer agent which is an antibody or an antigen-binding portion thereof that specifically binds to the PD-1 receptor and inhibits PD-1 activity (“an anti-PD-1 antibody or antigen-binding portion thereof”);
(b) a dosage of another anti-cancer agent which is a dosage ranging from 0.1 to 10 mg/kg body weight of an antibody or an antigen-binding portion thereof that specifically binds to and inhibits CTLA-4 (“an anti-CTLA-4 antibody or antigen-binding fragment thereof”); and
(c) instructions for using the anti-PD-1 antibody or antigen-binding fragment thereof and the anti-CTLA-4 antibody or antigen-binding fragment thereof in the method of claim 1.

16. The method of claim 1, wherein the anti-PD-1 antibody or antigen-binding portion thereof and the anti-CTLA-4 antibody or antigen-binding portion thereof are admixed as a single composition for concurrent administration.

17. The method of claim 1, wherein the anti-PD-1 antibody or antigen-binding portion thereof is administered at a flat dose of at least about 480 mg.

18. The method of claim 1, wherein the anti-PD-1 antibody or antigen-binding portion thereof is administered at a flat dose of about 480 mg once about every 4 weeks.

19. The method of claim 11, wherein the anti-PD-1 antibody or antigen-binding portion thereof is administered at a flat dose of at least about 480 mg.

20. The method of claim 11, wherein the anti-PD-1 antibody or antigen-binding portion thereof is administered at a flat dose of about 480 mg once about every 4 weeks.

21. The method of claim 1, wherein the anti-PD-1 antibody or antigen-binding portion thereof comprises nivolumab administered at a flat dose of about 240 mg once about every 2 weeks or at a flat dose of about 480 mg once about every 4 weeks.
